(19) United States
(12) Patent Application Publication
(10) Pub. No.: US 2021/0330786 A1
(43) Pub. Date: Oct. 28, 2021

(54) COMPOSITIONS COMPRISING BACTERIAL STRAINS

(71) Applicant: 4D PHARMA RESEARCH LIMITED, ABERDEEN (GB)

(72) Inventors: IMKE ELISABETH MULDER, ABERDEEN (GB); MARSILIO ADRIANI, ABERDEEN (GB); AMY BETH HOLT, ABERDEEN (GB); MARIA CHRISTOFI, ABERDEEN (GB); PHILIP COWIE, ABERDEEN (GB); EMMA RAFTIS, ABERDEEN (GB); DELPHINE LOUISE CLAUDETTE LAUTE-CALY, ABERDEEN (GB); EMMA ELIZABETH CLARE HENNESSY, ABERDEEN (GB); SUAAD AHMED, ABERDEEN (GB); ANNA ETTORRE, ABERDEEN (GB)

(21) Appl. No.: 17/176,791

(22) Filed: Feb. 16, 2021

Related U.S. Application Data
(63) Continuation of application No. PCT/EP2019/072181, filed on Aug. 19, 2019.

(30) Foreign Application Priority Data
Aug. 17, 2018 (EP) 18189521.0
Oct. 29, 2018 (GB) 1817648.7
Jan. 10, 2019 (GB) 1900335.9
Jan. 29, 2019 (GB) 1901203.8

(51) Int. Cl. A61K 39/39; A61K 35/745; A61P 37/04

(52) U.S. Cl. CPC A61K 39/39; A61K 2039/55594; A61P 37/04; A61K 35/745

(57) ABSTRACT

The invention provides a composition comprising a bacterial strain for use in stimulating the immune system in a subject.

### CROSS-REFERENCE

This application is a continuation of International Application No. PCT/EP2019/072181, filed Aug. 19, 2019, which claims the benefit of Great Britain Application No. 1817648.7, filed Oct. 29, 2018, European Application No. 18189521.0, filed Aug. 17, 2018, Great Britain Application No. 1900335.9, filed Jan. 10, 2019, and Great Britain Application No. 1901203.8, filed Jan. 29, 2019, all of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Jan. 14, 2021, is named 56708_746_301_SL.txt and is 4,096 bytes in size.

### TECHNICAL FIELD

This invention is in the field of compositions comprising bacterial strains isolated from the mammalian digestive tract and the use of such compositions in the treatment of disease, in particular in stimulating the immune system in the treatment of disease.

### BACKGROUND TO THE INVENTION

The human intestine is thought to be sterile in utero, but it is exposed to a large variety of maternal and environmental microbes immediately after birth. Thereafter, a dynamic period of microbial colonization and succession occurs, which is influenced by factors such as delivery mode, environment, diet and host genotype, all of which impact upon the composition of the gut microbiota, particularly during early life. Subsequently, the microbiota stabilizes and becomes adult-like [1]. The human gut microbiota contains more than 500-1000 different phylotypes belonging essentially to two major bacterial divisions, the Bacteroidetes and the Firmicutes [2]. The successful symbiotic relationships arising from bacterial colonization of the human gut have yielded a wide variety of metabolic, structural, protective and other beneficial functions. The enhanced metabolic activities of the colonized gut ensure that otherwise indigestible dietary components are degraded with release of by-products providing an important nutrient source for the host. Similarly, the immunological importance of the gut microbiota is well-recognized and is exemplified in germfree animals which have an impaired immune system that is functionally reconstituted following the introduction of commensal bacteria [3-5].

Dramatic changes in microbiota composition have been documented in gastrointestinal disorders such as inflammatory bowel disease (IBD). For example, the levels of Clostridium cluster XIVa bacteria are reduced in IBD patients whilst numbers of E. coli are increased, suggesting a shift in the balance of symbionts and pathobionts within the gut [6-9]. Interestingly, this microbial dysbiosis is also associated with imbalances in T effector cell populations.

In recognition of the potential positive effect that certain bacterial strains may have on the animal gut, various strains have been proposed for use in the treatment of various diseases (see, for example, [10-13]). Also, certain strains, including mostly Lactobacillus and Bifidobacterium strains, have been proposed for use in treating various inflammatory and autoimmune diseases that are not directly linked to the intestines, for example through anti-inflammatory mechanisms (see [14] and [15] for reviews). Certain Streptococcus and Veillonella strains, and to a lesser extent, Enterococcus and Lactobaccillus strains have been suggested to have immunomodulatory effects, with varying effects on different cytokines in vitro. However, the relationship between different diseases and different bacterial strains, and the precise effects of particular bacterial strains on the gut and at a systemic level and on any particular types of diseases, are poorly characterised.

Recently, various Bifidobacterium species have been investigated for their anti-inflammatory properties and several human trials have demonstrated that the therapeutic properties of bifidobacteria can be successfully translated from lab to clinic, in particular for inflammatory and autoimmune disorders. For instance, B. longum CECT 7347 and B. infantis NLS have both elicited protective effects in patients with coeliac disease [16,17]). Studies have also demonstrated the ability of bifidobacteria to modulate diseases beyond the gut. A three-strain formulation composed of B. longum BB536, B. infantis M-63, and B. breve M-16V alleviated the symptoms associated with allergic rhinitis and mild intermittent asthma in children [18]. Furthermore, administration of B. longum 35624 to patients with ulcerative colitis, psoriasis, and chronic fatigue syndrome resulted in decreased plasma levels of C-reactive protein in all three disorders [19], suggesting that this strain might be capable of modulating systemic immunity.

Organisms from the Bifidobacterium breve species have been proposed for use in preparing immunostimulatory supplements by metabolising linoleic acid in, for example, [20] and [21]. However, there is nothing in those documents to suggest that such organisms, when administered to subjects, could elicit an immunostimulatory response.

Reference [22] suggests that nutritional compositions comprising betα-galacto-oligosaccharides A and B may elicit an immunostimulatory effect. It is stated that compositions comprising those sugars may additionally comprise various other components, including Bifidobacterium breve, but there is no suggestion that Bifidobacterium breve organisms would contribute to or enhance any immunostimulatory properties of the betα-galacto-oligosaccharides.

The therapeutic use of organisms from the species Bifidobacterium breve is proposed in [23]. However, there is no suggestion that the bacterial strains disclosed therein had an immunostimulatory effect.

A study to investigate the potential effect of a strain of Bifidobacterium on immunogenicity of an oral cholera vaccine was reported in [24]. However, the outcome of that study was inconclusive; it was reported in that paper that the tested strain while being well tolerated, did not exhibit an evident post-vaccinal immunostimulatory effect.

There is a requirement in the art for new methods of treating diseases. There is also a requirement for the potential effects of gut bacteria to be characterised so that new therapies using gut bacteria can be developed.

### SUMMARY OF THE INVENTION

The inventors have developed new compositions comprising a bacterial strain of the species Bifidobacterium breve that can be used in stimulating the immune system and treating and preventing disease in a subject. The inventors have identified that strains of the species Bifidobacterium breve can potently activate the immune system.

The invention therefore provides a composition comprising a bacterial strain of the species Bifidobacterium breve, for use in stimulating the immune system in subject. Preferably, the invention provides a composition comprising the strain deposited under accession number 42380 at NCIMB, or a derivative or biotype thereof, for use in stimulating the immune system.

Additionally, the invention provides a method of stimulating the immune system, comprising administering a composition comprising a bacterial strain of the species Bifidobacterium breve to the subject. Furthermore, the invention provides a use of a composition comprising a bacterial strain of the species Bifidobacterium breve for the manufacture of a medicament for stimulating the immune system in a subject.

In further aspects, the invention provides a composition comprising a bacterial strain of the species Bifidobacterium breve, for use as a vaccine adjuvant. Preferably, the invention provides a composition comprising the strain deposited under accession number 42380 at NCIMB, or a derivative or biotype thereof, for use as a vaccine adjuvant.

In further aspects, the invention provides a composition comprising a bacterial strain of the species Bifidobacterium breve, for use in treating, preventing or delaying immunosenescence. Preferably, the invention provides a composition comprising the strain deposited under accession number 42380 at NCIMB, or a derivative or biotype thereof, for use in treating, preventing or delaying immunosenescence.

In further aspects, the invention provides a composition comprising a bacterial strain of the species Bifidobacterium breve, for use in enhancing a cell therapy, such as CAR-T. Preferably, the invention provides a composition comprising the strain deposited under accession number 42380 at NCIMB, or a derivative or biotype thereof, for use in enhancing a cell therapy, such as CAR-T.

The inventors have also characterised a strain of Bifidobacterium breve that is particularly potent at stimulating the immune system and have identified that its potency may be mediated by its exopolysaccharide (EPS). The invention therefore preferably uses a composition comprising a bacterial strain of the species Bifidobacterium breve that comprises a complete EPS locus and/or expresses EPS on its surface, for stimulating the immune system in subject.

Preferably, the bacteria used in the invention is the strain deposited under accession number 42380 at NCIMB.

In preferred embodiments, the invention provides a composition, for use in increasing the expression level and/or activity of IL-12p70, IL-12p70, IFNγ, IL-4 and/or TNF-α in the treatment or prevention of disease, as demonstrated in the examples. Preferably, the invention provides a composition comprising the strain deposited under accession number 42380 at NCIMB, or a derivative or biotype thereof, for use in increasing the expression level and/or activity of IL-12p70, IL-12p70, IFNγ, IL-4 and/or TNF-α in the treatment or prevention of disease.

In preferred embodiments, the invention provides a composition, for use in increasing the expression level and/or activity of IL-12p70, IL-12p70, IFNγ, IL-4, TNF-α and/or IL-17a in the treatment or prevention of disease, as demonstrated in the examples. Preferably, the invention provides a composition comprising the strain deposited under accession number 42380 at NCIMB, or a derivative or biotype thereof, for use in increasing the expression level and/or activity of IL-12p70, IL-12p70, IFNγ, IL-4, TNF-α and/or IL-17α in the treatment or prevention of disease.

In preferred embodiments, the invention provides a composition, for use in stimulating TLR2. Preferably, the invention provides a composition comprising the strain deposited under accession number 42380 at NCIMB, or a derivative or biotype thereof, for use in stimulating TLR2.

In preferred embodiments, the invention provides a composition, for use in stimulating NFκB. Preferably, the invention provides a composition comprising the strain deposited under accession number 42380 at NCIMB, or a derivative or biotype thereof, for use in stimulating NFκB.

In preferred embodiments, the bacterial strain of the invention expresses pullulanase, which is shown in the examples to be highly expressed by potent B. breve strains and may be involved in adhesion.

In further aspects, the invention provides a composition comprising a bacterial strain of the species Bifidobacterium breve for treating or preventing bacterial infections in a subject. The examples demonstrate that B. breve, and particularly the B. breve strains of the invention, have potent anti-microbial activity. Additionally, the invention provides a method of treating or preventing bacterial infections in a subject, comprising administering a composition a bacterial strain of the species Bifidobacterium breve. Furthermore, the invention provides a use of a composition comprising a bacterial strain of the species Bifidobacterium breve for the manufacture of a medicament for treating or preventing bacterial infections in a subject.

In preferred embodiments, the infection is a gastro-intestinal infection. In preferred embodiments, the infection is a Gram-negative bacterial infection. In preferred embodiments, the composition of the invention is for use in treating or preventing gastrointestinal E. coli infection. In preferred embodiments, the composition of the invention is for use in treating or preventing gastrointestinal S. enterica infection. In some embodiments, the composition of the invention is for use in reducing the viability of a bacteria in the treatment of a bacterial infection. The bacteria of the invention may be used to restore the level of pathogenic bacteria to asymptomatic levels or to eliminate the pathogenic bacteria entirely from a subject, thereby treating the bacterial infection, in addition to alleviating the symptoms associated with the elevated level of the bacteria.

Strains closely related to the B. breve strain tested in the examples are expected to be particularly effective at stimulating the immune system. In preferred embodiments, the invention provides a composition wherein the bacterial strain has a 16s rRNA gene sequence that is at least 95%, 96%, 97%, 98%, 99%, 99.5% or 99.9% identical to SEQ ID NO:1 or wherein the bacterial strain has a 16s rRNA gene sequence represented by SEQ ID NO:1.

In certain embodiments, the composition of the invention is for oral administration. Oral administration of the strains of the invention may be effective for stimulating the immune system. Also, oral administration is convenient for patients and practitioners and allows delivery to and/or partial or total colonisation of the intestine.

In certain embodiments, the composition of the invention comprises one or more pharmaceutically acceptable excipients or carriers.

In certain embodiments, the composition of the invention comprises a bacterial strain that has been lyophilised. The composition of the invention can also comprise a lyophilised bacteria strain of the species Bifidobacterium breve. Lyophilisation is an effective and convenient technique for preparing stable compositions that allow delivery of bacteria.

In certain embodiments, the invention provides a food product comprising the composition as described above. The invention also provides a food product comprising a bacterial strain of the species Bifidobacterium breve as described herein.

In certain embodiments, the invention provides a vaccine composition comprising a bacterial strain as described above. The invention also provides a vaccine composition comprising a composition according to the invention.

Additionally, the invention provides a method of treating or preventing a disease or condition associated with reduced immunostimulation, comprising administering a composition comprising a bacterial strain of the species Bifidobacterium breve to a patient in need thereof.

### BRIEF DESCRIPTION OF DRAWINGS

FIG. 1: Mouse model of breast cancer—tumor volume.

FIG. 2: Mouse model of lung cancer—tumour volume.

FIG. 3: Mouse model of liver cancer—liver weight.

FIGS. 4A-4B: Rapid ID 32 A profile of MRX004 alone (FIG. 4A) and in comparison with B. breve type strains (FIG. 4B). White=negative reaction (no colour change), Downwards cross-hatched=intermediate positive reaction (weak colour change) and Black=positive reaction (strong appropriate colour change).

FIG. 5: API® 50 CH analysis of MRX004. Upward cross-hatched=negative reaction (no colour change), Downward cross-hatch=intermediate positive reaction (weak colour change), Black=positive reaction (strong appropriate colour change) and White=doubtful reaction (unexpected colour change).

FIG. 6: Attachment of MRX004 and B. breve type strains to human cells.

FIGS. 7A-7B: Stimulation of NFκB and TLR2 by MRx0004 treatments. THP-1-NFκB (FIG. 7A) and HEK-TLR2 (FIG. 7B) reporter cell lines were treated with treatments of live MRx0004 (MRx0004_{LV}), MRx0004 culture supernatant (MRx0004_{SN}) and heat-inactivated MRx0004 (MRx0004_{HK}) at MOI 100:1 for 22 hours. Data are representative of three biological replicates. Statistical analysis was carried out using ordinary one-way ANOVA and Tukey's Multiple Comparisons Test. Statistically significant differences are presented as *p<0.05, ***p<0.001 and ****p<0.0001.

FIG. 8: Transcriptional response of MRx0004 in vitro and in response to IECs. The expression of ten MRx0004 genes was analysed and compared between cultures in late log phase growth and after contact (3 hours) with IECs. Data are presented as the fold change (2^{-ΔΔCt}) value calculated between named conditions and normalised to groEL, and are representative of three independent biological replicates. Statistical analysis was carried out using ordinary one-way ANOVA and Tukey's Multiple Comparisons Test. *p<0.05, **p<0.01, ***p<0.001, ****p<0.0001.

FIGS. 9A-9B: Sequence diversity within EPS loci of MRx0004 and related strains

FIGS. 10A-10C: Phenotypic properties of an EPS-negative derivative strain of MRx0004. Transmission electron microscopy (TEM) was carried out on MRx0004 (FIG. 10A) and its EPS-negative strain (EPS^{neg}) (FIG. 10B). (FIG. 10C) Bacterial adhesion to IECs was analysed after co-incubating HT29-MTX cells and bacterial strains at MOI 100:1 for 3 hours. Data presented are the average of three biological replicates of the percentage of adherent CFU from the initial inoculum. Statistical analysis was carried out using ordinary one-way ANOVA and Tukey's Multiple Comparisons Test. *p<0.05, **p<0.01.

FIGS. 11A-11C: Impact of EPS depletion on MRx0004 surface protein detection. Venn diagram showing the number of proteins identified in (FIG. 11A) MRx0004 shaved and shed proteins fractions, (FIG. 11B) EPS^{neg} shaved and shed proteins fractions and (FIG. 11C) MRx0004 and EPS^{neg} shaved protein fractions.

FIGS. 12A-12C. Elucidating the role of EPS in immunomodulation by MRx0004. Live treatments (_{LV}) and culture supernatants (_{SN}) were added to HEK-TLR2 reporter cells (FIG. 12A) and THP-1-NFκB reporter cells (FIG. 12B) at an MOI of 100:1, and incubated for 22 hours. Data presented for MRx0004_{L}v and MRx0004_{SN} have been previously presented in FIG. 7. Data are representative of three independent replicates. (FIG. 12C) HT29-MTX cells were co-incubated with live bacteria for 3 hours, following which TNFαwas introduced at a concentration of 10 ng/ml for 24 hours. IL-8 levels were analysed in co-culture supernatants using ELISA. Data are representative of five biological replicates. Statistical analysis was carried out using ordinary one-way ANOVA and Tukey's Multiple Comparisons Test. *p<0.05, **p<0.01, ***p<0.001, ****p<0.0001.

FIGS. 13A-13E: Identification of immune cell subsets by flow cytometry. PBMCs from six healthy donors were incubated for 72 hours with one of the following treatments; RPMI media alone, MRx0004_{HK}, or EPS^{neg}_{HK}. Box and whisker plots are shown with their minimum and maximum represented by vertical whiskers. Expression of the cellular activation marker CD25 is shown as a percentage of CD8⁺ and CD4⁺ cells (FIG. 13A-13B). Tregs (CD25⁺ (high)/CD127⁻(low)) are shown as a percentage of CD4⁺ cells (FIG. 13C). Independent Treg/CD8⁺ ratios were calculated from each donor (FIG. 13D) and B-cells (CD19⁺) as a percentage of CD3⁻ cells (FIG. 13E). and statistical analysis was carried out using ordinary one-way ANOVA followed by Tukey's multiple comparisons test. *p<0.05, **p<0.01* **p<0.001****p<0.0001.

FIGS. 14A-14J: Cytokine profiles produced by peripheral blood mononuclear cells (PBMCs). Cells were incubated for 72 hours with one of the following treatments; RPMI media alone, MRx0004_{HK}, or EPS^{neg}_{HK}. Box and whisker plots are shown with their minimum and maximum represented by vertical whiskers. PBMCs were obtained from 6 healthy donors and cytokine concentrations were determined using a multiplex assay (FIGS. 14A-14G). Independent IL-12p70/IL-4, IL-10/IL1p70 and IL-1β/IL12p70 ratios were calculated from each donor (FIGS. 14H-14J) and statistical analysis was carried out using ordinary one-way ANOVA followed by Tukey's multiple comparisons test. *p<0.05, **p<0.01***p<0.001****p<0.0001.

FIG. 15: Venn diagram, generated with InteractiVenn, showing the number of proteins identified in MRx0004 shaved and shed proteins fractions (listed in Table 2).

FIGS. 16A-16B: (FIG. 16A) Generation of an EPS negative strain of MRx0004 by insertional mutagenesis. (FIG. 16B) Autoaggregation of MRx0004 and its derivative strains EPS^{neg}, EPS^{vec} and EPS^{comp}.

FIG. 17: Venn diagram comparing the number of proteins identified in MRx0004SN and EPSnegSN.

FIGS. 18A-18F: Gating strategy used for seven colour panel flow cytometry experiment. An acquisition threshold was set using the live cell population at 1×10⁵ events. Shown are representative pseudocolour plots of human peripheral blood mononuclear cells (PBMCs) from an untreated sample. Gates were set using isotype controls and FMOs in FloJo. Forward and side scatter were used to identify lymphocytes before gating out the doublet population and utilising a viability dye to exclude dead cells. CD3⁻cells were sub-gated on with CD19 to identify B-cells, whilst the CD3⁺cell population was used to further distinguish both the CD4⁺ and CD8⁺ cells. CD25 was then used to look at the percentage of activated cells and in the case of the CD4⁺ population it was also used in conjunction with CD127 to identify Tregs (CD25⁺/CD127⁻).

FIGS. 19A-19C: Identification of immune cell subsets by flow cytometry. PBMCs from six healthy donors were incubated for 72 hours with one of the following treatments; RPMI media alone, MRx0004_{HK}, or EPS^{neg}_{HK}. Box and whisker plots are shown with their minimum and maximum represented by vertical whiskers. CD8⁺ and CD4⁺ cells are shown as a percentage of CD3⁺ (FIG. 19A-FIG 19B) and activated B-cells as a percentage of CD19⁺ (FIG. 19C). Statistical analysis was carried out using ordinary one-way ANOVA followed by Tukey's multiple comparisons test. *p <0.05, **p<0.01***p<0.001****p<0.0001.

FIGS. 20A-20H: Identification of immune cell subsets by flow cytometry. PBMCs from six healthy donors were incubated for 72 hours with one of the following treatments; MRx0004_{HK}, EPS^{neg}_{HK}, EPS^{vec}_{HK} or EPS^{comp}_{HK}. Scatter plots are shown with their standard deviations represented by vertical bars. Expression of CD8⁺ and CD4⁺cells are shown as a percentage of CD3^{'} cells (FIGS. 20A, 20C). Expression of the cellular activation marker CD25 is shown as a percentage of CD4⁺ and CD8⁺ cells (FIGS. 20B, 20D). Tregs (CD25⁺ (high)/CD127⁻(low)) are shown as a percentage of CD4⁺ cells (FIG. 20E) and B-cells (CD19⁺) as a percentage of CD3⁻cells (FIG. 20F) and activated B-cells as a percentage of CD19⁺ (FIG. 20G). Independent Treg/CD8⁺ ratios were calculated from each donor (FIG. 20H) and statistical analysis was carried out using ordinary one-way ANOVA followed by Tukey's multiple comparisons test. *p<0.05, **p<0.01***p<0.001****p<0.0001.

FIGS. 21A-21J: Cytokine profiles produced by peripheral blood mononuclear cells (PBMCs). Cells were incubated for 72 hours with one of the following treatments; MRx0004_{HK} EPS^{neg}_{HK}, EPS^{vec}_{HK} or EPS^{comp}_{HK}. Box and whisker plots are shown with their minimum and maximum represented by vertical whiskers. PBMCs were obtained from 6 healthy donors and cytokine concentrations were determined using a multiplex assay (FIGS. 21A-21G). Independent IL-12p70/IL-4, IL-10/IL1p70 and IL-1β/IL12p70 ratios were calculated from each donor (FIGS. 21H-21J). Statistical analysis was carried out using ordinary one-way ANOVA followed by Tukey's multiple comparisons test. *p<0.05, **p<0.01***p<0.001****p<0.0001.

FIG. 22: Example of a co-culture antimicrobial plate assay. Shown are the indicator strain, the test strains and the inhibition zone.

FIG. 23: API 32A test results for the test and reference B. breve strains. White=negative reaction (no colour change), Star=intermediate positive reaction (weak colour change) and Black=positive reaction (strong appropriate colour change).

FIG. 24: PFGE Spel Digest for the test and reference B. breve strains.

FIG. 25: Spel digested 1% PFGE gel run in 0.5% TBE, 18 hrs, 6 V/cm, 1-15 sec ST. Black arrows indicate DNA size standards. Lanes 1 and 2 were grouped with lanes 3-7 from different parts of the same gel. Lane 1=λ, Lane 2=MRx0004, Lane 3=B. breve REF 7, Lane 4=B. breve REF6, Lane 5=B. breve REF1, Lane 6=B. breve REF2, Lane 7=λ

FIGS. 26A-26B: UPGMA dendrogram based on the PFGE patterns of B. breve included in this study is shown in panel A (FIG. 26A). A similarity matrix generated from the PFGE banding pattern represented in panel B (FIG. 26B).

FIG. 27: Induction of T-cell differentiation in a population of T-helper cells using heat-killed MRx0004 (HK 4), supernatant from MRx0004 culture (SP 4) or RPMI medium, without addition of cytokines (no cyto). **=p≤0.01.

FIG. 28: Induction of T-cell differentiation in a population of Cytotoxic T Lymphocytes (CTL) using heat-killed MRx0004 (HK 4), supernatant from MRx0004 culture (SP 4) or RPMI medium, without addition of cytokines (no cyto). *=p≤0.05; ***=p≤0.001; ****=p≤0.0001.

FIG. 29: Viability of splenocytes.

FIG. 30: Cytokine profiles produced by splenocytes after treatment with MRx004.

FIG. 31: Frequency of CD8+IFNγ+ and CD4+IFNγ+ cells and per cell IFNγ production in spleen.

### DISCLOSURE OF THE INVENTION

Bacterial Strains

The compositions of the invention comprise a strain of the species Bifidobacterium breve. The examples demonstrate that such bacterial strains are useful for stimulating the immune system. The preferred bacterial strain of the invention is the bacterium deposited under accession number NCIMB 42380.

The Bifidobacterium breve bacterium deposited under accession number NCIMB 42380 was tested in the Examples and is also referred to herein as strain 751, MRX004 or MRx0004. A partial 16S rRNA sequence for the MRX004 strain that was tested is provided in SEQ ID NO:1. Bifidobacterium breve strain MRX004 was deposited with the international depositary authority NCIMB, Ltd. (Ferguson Building, Craibstone Estate, Bucksburn, Aberdeen, AB21 9YA, Scotland) by GT Biologics Ltd. (Life Sciences Innovation Building, Aberdeen, AB25 2ZS, Scotland) under identification reference 751 on 12 Mar. 2015 and was assigned accession number NCIMB 42380. GT Biologics Ltd. subsequently changed its name to 4D Pharma Research Limited. These deposits were published in WO2016/203223.

A genome sequence for strain NCIMB 42380 is provided in SEQ ID NO:2 of WO2016/203223.

Bacterial strains closely related to the strain tested in the examples are also expected to be effective for stimulating the immune system. In certain embodiments, the bacterial strain for use in the invention has a 16s rRNA sequence that is at least 95%, 96%, 97%, 98%, 99%, 99.5% or 99.9% identical to SEQ ID NO:1. Preferably, the bacterial strain has the 16s rRNA sequence represented by SEQ ID NO:1 . Most preferably, the bacterial strain is the Bifidobacterium breve strain deposited under accession number NCIMB 42380.

In certain embodiments, the bacterial strain for use in the invention has a genome with sequence identity to SEQ ID NO:2 of WO2016/203223. In preferred embodiments, the bacterial strain for use in the invention has a genome with at least 90% sequence identity (e.g. at least 92%, 94%, 95%, 96%, 97%, 98%, 99% or 100% sequence identity) to SEQ ID NO:2 of WO2016/203223 across at least 60% (e.g. at least 65%, 70%, 75%, 80%, 85%, 95%, 96%, 97%, 98%, 99% or 100%) of SEQ ID NO: 2 of WO2016/203223. For example, the bacterial strain for use in the invention may have a genome with at least 90% sequence identity to SEQ ID NO: 2 of WO2016/203223 across 70% of SEQ ID NO: 2 of WO2016/203223, or at least 90% sequence identity to SEQ ID NO: 2 of WO2016/203223 across 80% of SEQ ID NO: 2 of WO2016/203223, or at least 90% sequence identity to SEQ ID NO: 2 of WO2016/203223 across 90% of SEQ ID NO: 2 of WO2016/203223, or at least 90% sequence identity to SEQ ID NO: 2 of WO2016/203223 across 100% of SEQ ID NO: 2 of WO2016/203223, or at least 95% sequence identity to SEQ ID NO: 2 of WO2016/203223 across 70% of SEQ ID NO: 2 of WO2016/203223, or at least 95% sequence identity to SEQ ID NO: 2 of WO2016/203223 across 80% of SEQ ID NO: 2 of WO2016/203223, or at least 95% sequence identity to SEQ ID NO: 2 of WO2016/203223 across 90% of SEQ ID NO: 2 of WO2016/203223, or at least 95% sequence identity to SEQ ID NO: 2 of WO2016/203223 across 100% of SEQ ID NO: 2 of WO2016/203223, or at least 98% sequence identity to SEQ ID NO: 2 of WO2016/203223 across 70% of SEQ ID NO: 2 of WO2016/203223, or at least 98% sequence identity to SEQ ID NO: 2 of WO2016/203223 across 80% of SEQ ID NO: 2 of WO2016/203223, or at least 98% sequence identity to SEQ ID NO: 2 of WO2016/203223 across 90% of SEQ ID NO: 2 of WO2016/203223, or at least 98% sequence identity to SEQ ID NO: 2 of WO2016/203223 across 100% of SEQ ID NO: 2 of WO2016/203223.

In preferred embodiments, the composition of the invention comprises live bacteria. In preferred embodiments, the composition of the invention comprises live bacteria in an active state, preferably lyophilised. The examples demonstrate that administration of live bacteria is more effective than heat killed bacteria or supernatants.

In preferred embodiments, the bacteria of the invention activate TLR2, for example in a HEK-TLR2 reporter assay such as described in the examples. In further embodiments, the bacteria of the invention do not activate TLR4, TLRS or TLR9. In a preferred embodiment, the composition of the invention comprises a bacteria that activates TLR2 and is for use as a vaccine adjuvant. In a preferred embodiment, the composition of the invention comprises a bacteria that activates TLR2 and is for use in enhancing a cell therapy. In a preferred embodiment, the composition of the invention comprises a bacteria that activates TLR2 and is for use in treating, preventing or delaying immunosenescence.

In preferred embodiments, the bacteria of the invention activate NFκB, for example in a THP-1-NFκB reporter assay such as described in the examples. In a preferred embodiment, the composition of the invention comprises a bacteria that activates NFκB and is for use as a vaccine adjuvant. In a preferred embodiment, the composition of the invention comprises a bacteria that activates NFκB and is for use in enhancing a cell therapy. In a preferred embodiment, the composition of the invention comprises a bacteria that activates NFκB and is for use in treating, preventing or delaying immunosenescence.

In certain embodiments, the bacteria of the invention express one or more genes selected from the group consisting of: oppA, pullulanase, serpin and tadA at a higher level in stationary phase compared to late log phase, for example, when grown in liquid culture, such as shown in the examples. The examples demonstrate that these genes may mediate useful therapeutic effects.

In certain embodiments, the bacteria of the invention express one or more genes selected from the group consisting of: eftU, enolase and pGTF at a higher level in late log phase compared to stationary phase, for example, when grown in liquid culture, such as shown in the examples. The examples demonstrate that these genes may mediate useful therapeutic effects.

In certain embodiments, the bacteria of the invention express one or more genes selected from the group consisting of: enolase, pGTF, oppA, serpin and transaldolase at a higher level after contact with intestinal epithelial cells compared to late log phase, for example, when grown in liquid culture, such as shown in the examples. The examples demonstrate that these genes may mediate useful therapeutic effects.

In certain embodiments, the bacteria of the invention expresses and secretes into the culture supernatant one or more of pullulanase, NlpC/P60 family proteins, FtsI, transaldolase, GAPDH, DnaK, GroEL and enolase. In certain embodiments, the bacteria of the invention expresses and secretes into the culture supernatant one or more, such as 2, 3, 5, 10, 15, or all, of the proteins in Table 2. The examples demonstrate that these proteins may mediate useful therapeutic effects.

In certain embodiments, the bacteria of the invention expresses on its cell surface one or more of pullulanase, a type I polyketide synthase, transaldolase, GAPDH, DnaK, GroEL, enolase and EfTu. In certain embodiments, the bacteria of the invention expresses and secretes into the culture supernatant one or more, such as 2, 3, 5, 6, 8, or all, of the proteins in Table 3. The examples demonstrate that these proteins may mediate useful therapeutic effects.

In certain embodiments, the bacteria of the invention express pullulanase. In a preferred embodiment, the composition of the invention comprises a bacteria that expresses pullulanase and is for use as a vaccine adjuvant. In a preferred embodiment, the composition of the invention comprises a bacteria that expresses pullulanase and is for use in enhancing a cell therapy. In a preferred embodiment, the composition of the invention comprises a bacteria that expresses pullulanase and is for use in treating, preventing or delaying immunosenescence.

In preferred embodiments, the bacteria of the invention comprises a complete EPS locus. The examples demonstrate that a complete EPS locus may contribute to increased therapeutic potency. In such embodiments, the EPS locus comprises a priming glycosyltransferase, one or more additional glycosyltransferases, a thiamine pyrophosphate binding protein, a membrane spanning protein, a flippase and a chain-length determinant. In preferred embodiments, the EPS locus is over 30 Kb in size (including flanking hypothetical proteins). The examples demonstrate that such an EPS locus is adequate for immunostimulatory function. In preferred embodiments, the EPS locus is 25-60 Kb, 30-50 Kb, 30-40 Kb, 30-35 Kb, or 30-32 Kb in size. In a preferred embodiment, the composition of the invention comprises a bacteria with a complete EPS locus and is for use as a vaccine adjuvant. In a preferred embodiment, the composition of the invention comprises a bacteria with a complete EPS locus and is for use in enhancing a cell therapy. In a preferred embodiment, the composition of the invention comprises a bacteria with a complete EPS locus and is for use in treating, preventing or delaying immunosenescence.

In preferred embodiments, the bacteria of the invention has a EPS locus with a high level of nucleotide identity to the EPS locus of strain MRX004, such as at least 90, 92, 94, 96, 98, 99 or 99.5% nucleotide identity, for example as determined in the examples. The examples demonstrate that the EPS locus of strain MRX004 is genetically distinct from other B. breve strains and may contribute to potency and therapeutic utility.

In preferred embodiments, the bacteria of the invention carries EPS on its surface. The examples demonstrate that EPS modulates the exposure of proteins on the cell surface. In a preferred embodiment, the composition of the invention comprises a bacteria that carries EPS on its surface and is for use as a vaccine adjuvant. In a preferred embodiment, the composition of the invention comprises a bacteria that carries EPS on its surface and is for use in enhancing a cell therapy. In a preferred embodiment, the composition of the invention comprises a bacteria that carries EPS on its surface and is for use in treating, preventing or delaying immunosenescence.

Preferably, the bacteria used in the invention is able to ferment raffinose, for example when cultured in an appropriate suspension medium (such as API suspension medium) at 37° C. for 4 hours. The examples suggest that the most effective B. breve strains are able to ferment raffinose, and it is involved in EPS production. In further preferred embodiments, the bacteria used in the invention is able to ferment one or more, such as 2, 3, 4, 5, 6 or all 7 of: α-galactosidase, β-galactosidase, α-glucosidase and β-glucosidase, α-arabinose, mannose and raffinose, for example when cultured in an appropriate suspension medium (such as API suspension medium) at 37° C. for 4 hours. In further preferred embodiments, the bacteria used in the invention is able to ferment one or more, such as 2, 3, 4, 5, 6 or all 7 of: arginine, proline, phenylalanine, leucine, tyrosine, glycine and histidine. Any suitable assay known in the art may be used to assess the ability of a bacterium to ferment a carbohydrate source or amino acid. Preferebly, the Rapid ID 32A analysis is used (preferably using the Rapid ID 32A system from bioMérieux).

Preferably, the bacteria used in the invention exhibit intermediate fermentation of β-glucosidase or intermediate fermentation of α-arabinose, or more preferably intermediate fermentation of β-glucosidase and intermediate fermentation of α-arabinose, for example when cultured in an appropriate suspension medium (such as API suspension medium) at 37° C. for 4 hours, and for example when subjected to the Rapid ID 32A analysis. The examples demonstrate that both B. breve strains MRX004 and Test 3 have useful activity and both exhibit intermediate fermentation of β-glucosidase and α-arabinose. In particularly preferred embodiments, the bacteria used in the invention exhibit intermediate fermentation of β-glucosidase, intermediate fermentation of α-arabinose, and positive fermentation of rabinose.

Preferably, the bacteria used in the invention do not exhibit positive fermentation of N-acetyl-β-glucosaminidase, for example when cultured in an appropriate suspension medium (such as API suspension medium) at 37° C. for 4 hours, and for example when subjected to the Rapid ID 32A analysis. The bacteria may exhibit only intermediate or no fermentation of N-acetyl-β-glucosaminidase. The examples demonstrate that both B. breve strains MRX004 and Test 2 have useful activity and neither exhibits positive fermentation of N-acetyl-β-glucosaminidase. In particularly preferred embodiments, the bacteria used in the invention do not exhibit positive fermentation of N-acetyl-β-glucosaminidase and do exhibit positive fermentation of rabinose.

Preferably, the bacteria used in the invention exhibit intermediate fermentation of α-galactosidase or intermediate fermentation of α-arabinose, or more preferably intermediate fermentation of α-galactosidase and intermediate fermentation of α-arabinose, for example when cultured in an appropriate suspension medium (such as API suspension medium) at 37° C. for 4 hours, and for example when subjected to the Rapid ID 32A analysis. The examples demonstrate that B. breve strains MRX004 and Test 8 both have useful activity and both exhibit intermediate fermentation of α-galactosidase and α-arabinose. In particularly preferred embodiments, the bacteria used in the invention exhibit intermediate fermentation of α-galactosidase and intermediate fermentation of α-arabinose, and positive fermentation of rabinose.

In alternative embodiments, the bacteria used in the invention ferment serine arylamidase but not leucyl glycine arylamidase and not alanine arylamidase, for example when cultured in an appropriate suspension medium (such as API suspension medium) at 37° C. for 4 hours, and for example when subjected to the Rapid ID 32A analysis. The examples demonstrate that B. breve strains Test 11 and Test 12 both have useful activity and both ferment serine arylamidase but not leucyl glycine arylamidase and not alanine arylamidase. In particularly preferred embodiments, the bacteria used in the invention also ferment rabinose.

In alternative embodiments, the bacteria used in the invention exhibit intermediate fermentation of serine arylamidase, for example when cultured in an appropriate suspension medium (such as API suspension medium) at 37° C. for 4 hours, and for example when subjected to the Rapid ID 32A analysis. The examples demonstrate that B. breve strains Test 3 and Test 7 both have potent anti-microbial activity and both exhibit intermediate fermentation of serine arylamidase. In particularly preferred embodiments, the bacteria used in the invention also ferment rabinose.

Any suitable assay known in the art may be used to assess the ability of a bacterium to ferment a carbohydrate source or amino acid. Preferebly, the Rapid ID 32A analysis is used (preferably using the Rapid ID 32A system from bioMérieux).

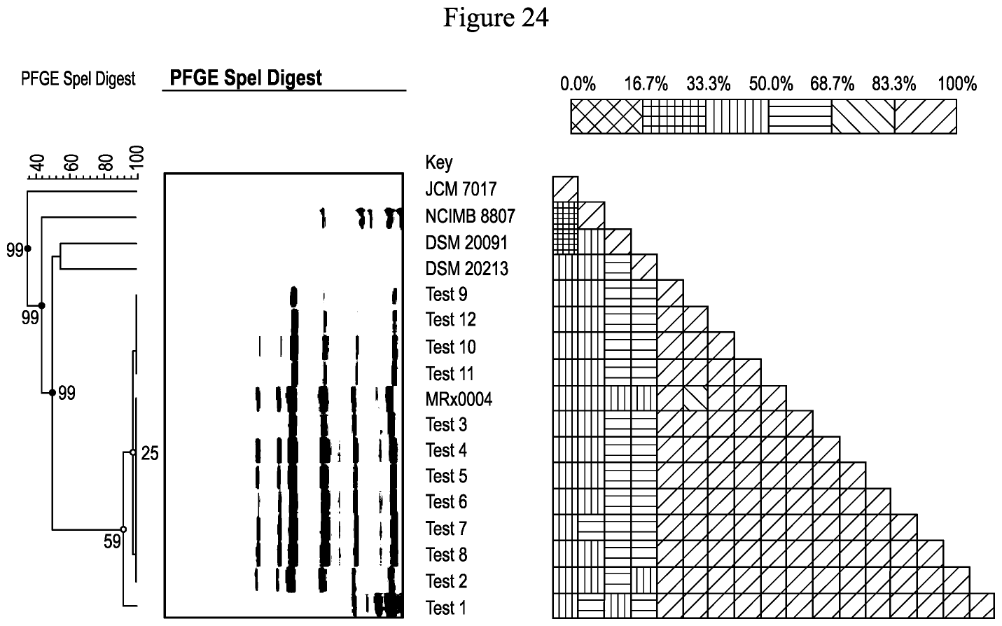

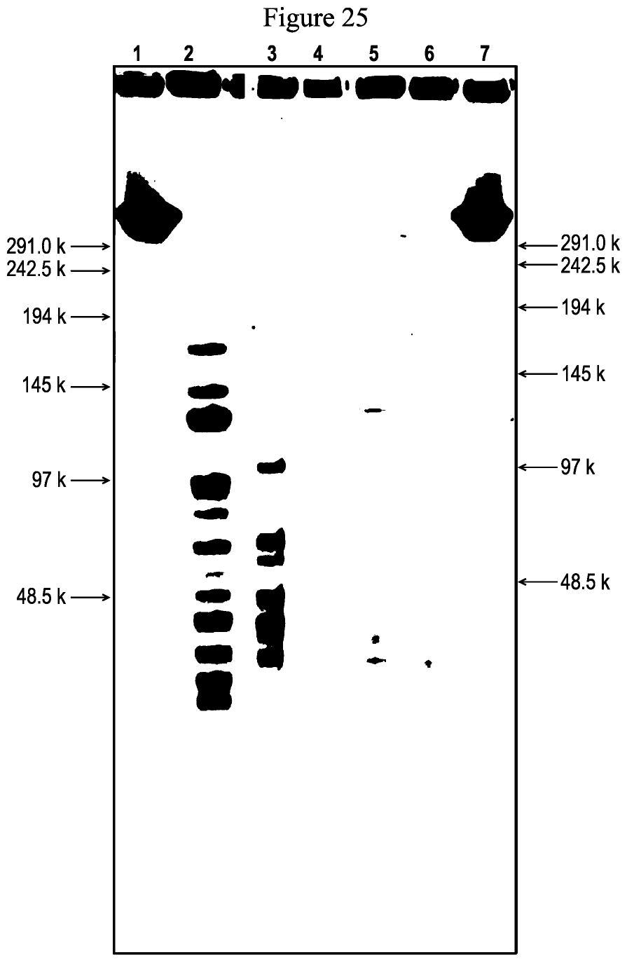

Preferably, the bacteria used in the invention produce the pattern shown in FIG. 24 or FIG. 25 for MRX004 when subjected to pulsed-field gel electrophoresis using standard conditions, such as those used in Example 10.

In alternative preferred embodiments, the bacteria used in the invention is able to ferment one or more, such as 2, 3, 4, 5, 10, 15, 20, 25 or all of: amidon (starch), amygdalin, arbutin, cellobiose, esculin, galactose, gentiobiose, glucose, glycogen, fructose, fucose, lactose, maltose, mannose, mannitol, melibiose, melezitose, methyl a-D-glucopyranoside, N-acetylglucosamine, ribose, saccharose (sucrose), salicin, sorbitol, trehalose, turanose and xylitol. In such embodiments, any suitable assay known in the art may be used to assess the ability of a bacterium to ferment a carbohydrate source. Preferebly, the API 50 CH analysis is used from bioMérieux.

In certain embodiments, the compositions of the invention comprise a strain of Bifidobacterium breve that exhibits reduced attachment to human cells, in particular when tested in YCFA medium, in particular under the conditions of Example 5.

In certain embodiments, a composition of the invention comprises a biotype of the bacterium deposited under accession number NCIMB 42380. Bacterial strains that are biotypes of the bacterium deposited under accession number NCIMB 42380 are also expected to be effective for stimulating the immune system. A biotype will have comparable immune modulatory activity to the original NCIMB 42380 strain. A biotype is a closely related strain that has the same or very physiological and biochemical characteristics.

A biotype will elicit comparable effects on the immune system to the effects shown in the examples, which may be identified by using the culturing and administration protocols described in the examples. In particular, a biotype will elicit an effect on T cells and cytokines comparable to NCIMB 42380.

Strains that are biotypes of a bacterium deposited under accession number NCIMB 42380 and that are suitable for use in the invention may be identified by sequencing other nucleotide sequences for a bacterium deposited under accession number NCIMB 42380. For example, substantially the whole genome may be sequenced and a biotype strain for use in the invention may have at least 95%, 96%, 97%, 98%, 99%, 99.5% or 99.9% sequence identity across at least 80% of its whole genome (e.g. across at least 85%, 90%, 95% or 99%, or across its whole genome). For example, in some embodiments, a biotype strain has at least 98% sequence identity across at least 98% of its genome or at least 99% sequence identity across 99% of its genome. Other suitable sequences for use in identifying biotype strains may include hsp60 or repetitive sequences such as BOX, ERIC, (GTG)₅, or REP [25].

Biotype strains may have such sequences with at least 95%, 96%, 97%, 98%, 99%, 99.5% or 99.9% sequence identity to the corresponding sequence of a bacterium deposited under accession number NCIMB 42380. In some embodiments, a biotype strain may have a 16S rRNA sequence with at least 95%, 96%, 97%, 98%, 99%, 99.5% or 99.9% sequence identity to the corresponding sequence of a bacterium deposited under accession number NCIMB 42380. In some embodiments, a biotype strain may comprises a 16S rRNA sequence that is at least 99% identical (e.g. at least 99.5% or at least 99.9% identical) to SEQ ID NO:1. In some embodiments, a biotype strain has the 16S rRNA sequence of SEQ ID NO:1.

Alternatively, strains that are biotypes of a bacterium deposited under accession number NCIMB 42380 and that are suitable for use in the invention may be identified by using the accession number NCIMB 42380 deposit, and restriction fragment analysis and/or PCR analysis, for example by using fluorescent amplified fragment length polymorphism (FAFLP) and repetitive DNA element (rep)-PCR fingerprinting, or protein profiling, or partial 16S or 23s rDNA sequencing. In preferred embodiments, such techniques may be used to identify other Bifidobacterium breve strains.

In certain embodiments, strains that are biotypes of a bacterium deposited under accession number NCIMB 42380 and that are suitable for use in the invention are strains that provide the same pattern as a bacterium deposited under accession number NCIMB 42380 when analysed by amplified ribosomal DNA restriction analysis (ARDRA), for example when using Sau3AI restriction enzyme (for exemplary methods and guidance see, for example [26]). Alternatively, biotype strains are identified as strains that have the same carbohydrate fermentation patterns as a bacterium deposited under accession number NCIMB 42380.

Other Bifidobacterium breve strains that are useful in the compositions and methods of the invention, such as biotypes of a bacterium deposited under accession number NCIMB 42380, may be identified using any appropriate method or strategy, including the assays described in the examples. In particular, bacterial strains that have similar growth patterns, metabolic type and/or surface antigens to a bacterium deposited under accession number NCIMB 42380 may be useful in the invention.

In certain embodiments, a composition of the invention comprises a derivative of the bacterium deposited under accession number NCIMB 42380. A derivative of the strain deposited under accession number NCIMB 42380 may be a daughter strain (progeny) or a strain cultured (subcloned) from the original. A derivative of a strain of the invention may be modified, for example at the genetic level, without ablating the biological activity. In particular, a derivative strain of the invention is therapeutically active. A derivative strain will have comparable immune modulatory activity to the original NCIMB 42380 strain. A derivative strain will have comparable microbiota modulatory activity to the original NCIMB 42380 strain. A derivative strain will therefore be effective in stimulating the immune system.

A derivative strain will elicit comparable effects cancer models to the effects shown in the examples, which may be identified by using the culturing and administration protocols described in the examples. In particular, a derivative strain will elicit an effect cytokines and gene expression comparable to those of a bacterium deposited under accession number NCIMB 42380. In particular, a derivative strain will elicit an effect on immune stimulation comparable to that of a bacterium deposited under accession number NCIMB 42380. A derivative of the NCIMB 42380 strain will generally be a biotype of the NCIMB 42380 strain.

The bacterial strain may also be a strain that has the same safety and therapeutic efficacy characteristics as the strain deposited under accession number NCIMB 42380, and such cells are encompassed by the invention. The composition can therefore comprise a Bifidobacterium breve strain that is not the strain deposited under accession number NCIMB 42380 but has the same safety and therapeutic efficacy characteristics as the strains deposited under accession number NCIMB 42380. The safety characteristics of a strain can be established for example by testing the resistance of the strain to antibiotics, for example distinguishing between intrinsic and transmissible resistance to antibiotics. The safety characteristics of a strain can also be established by evaluating the pathogenic properties of a strain in vitro, for example the levels of toxin production. Other safety tests include testing the acute or chronic toxicity of the bacterial strain in rat and mice models. The therapeutic efficacy of a strain can be established by functional characterization of the bacterial strain in vitro and in vivo using a relevant model.

In preferred embodiments, the bacterial strains in the compositions of the invention are viable and capable of partially or totally colonising the intestine.

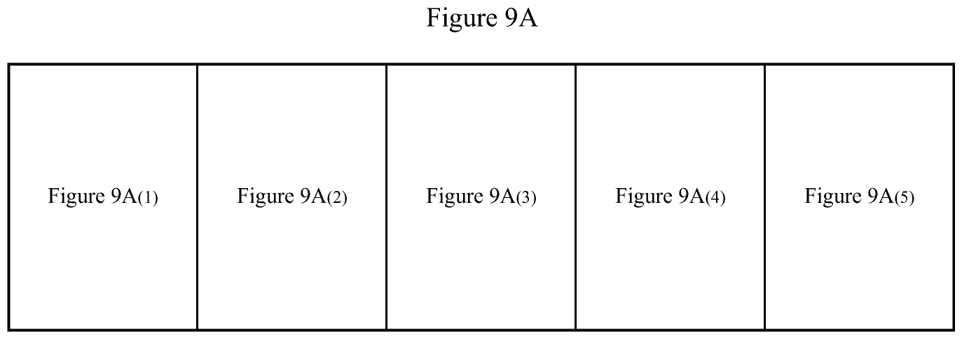

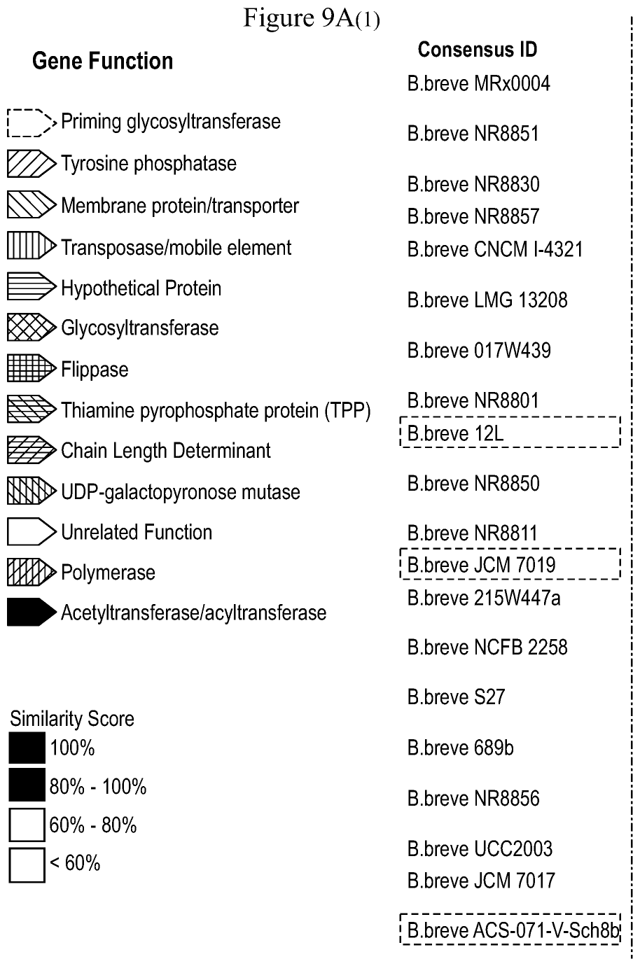

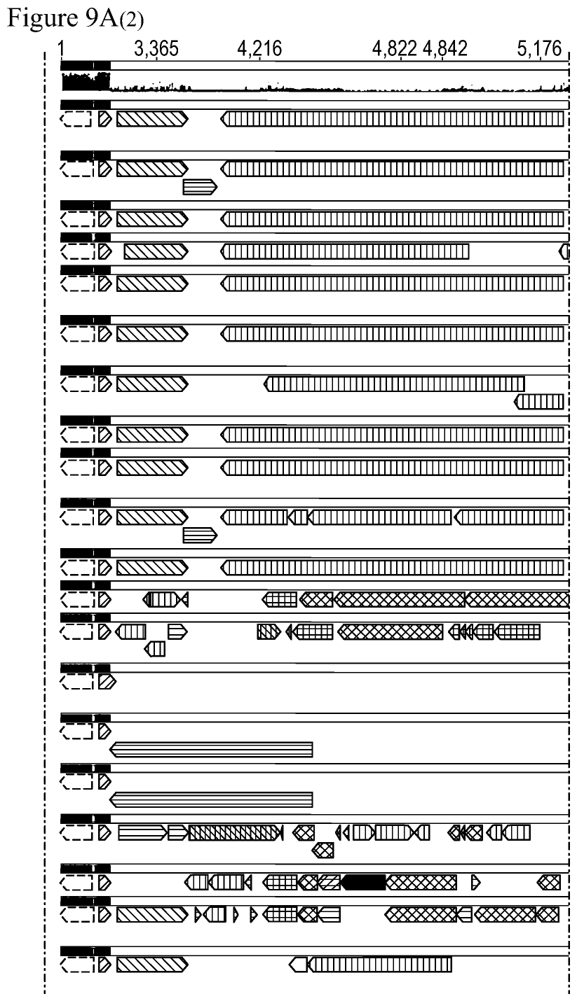

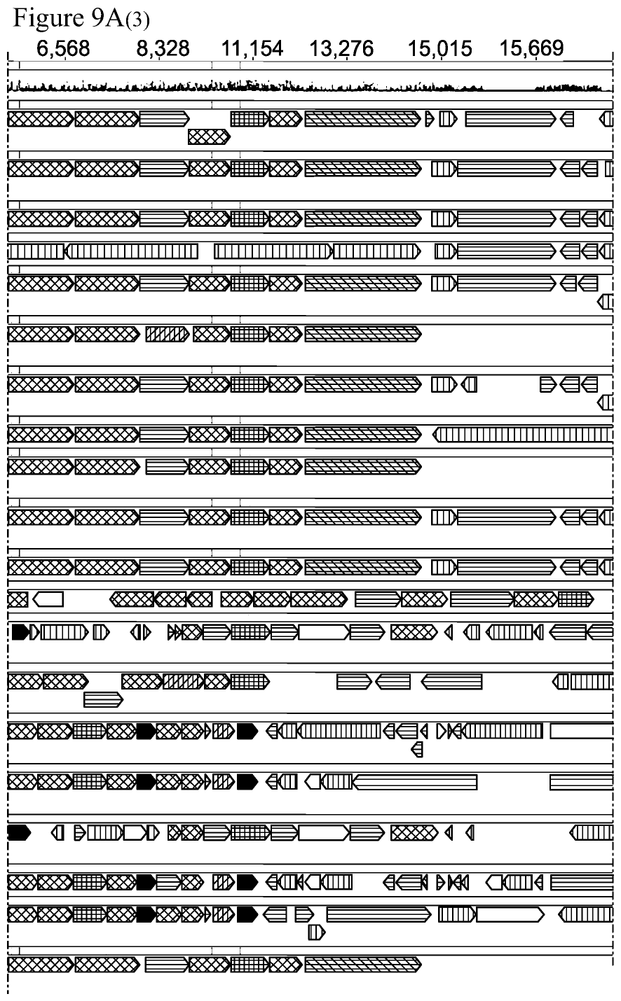

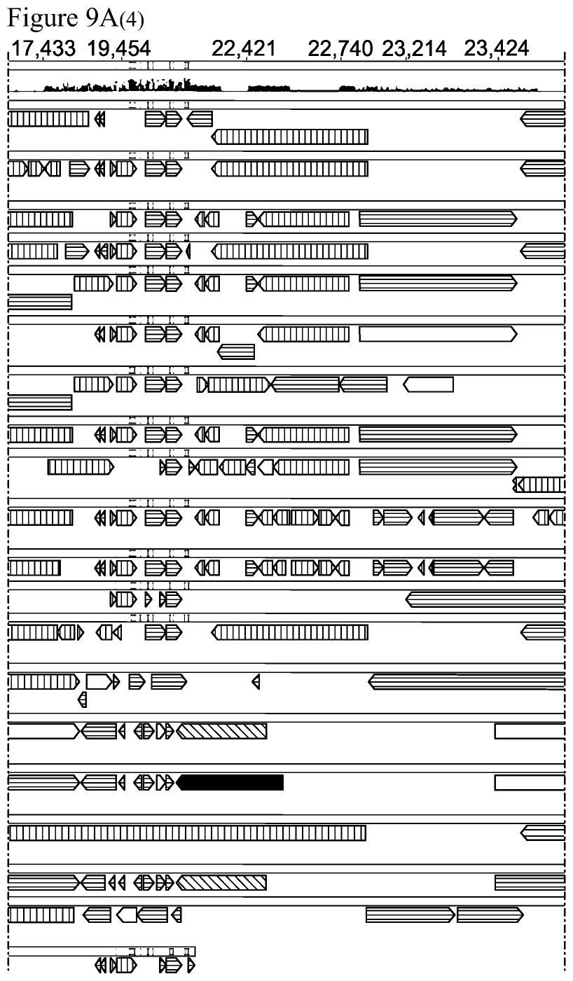

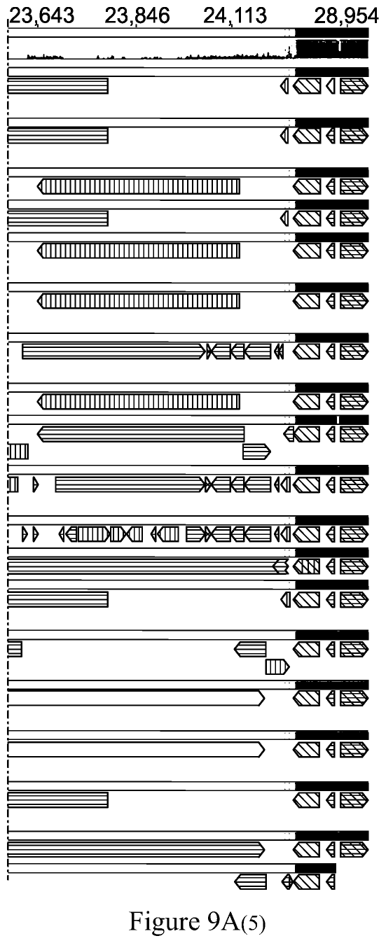

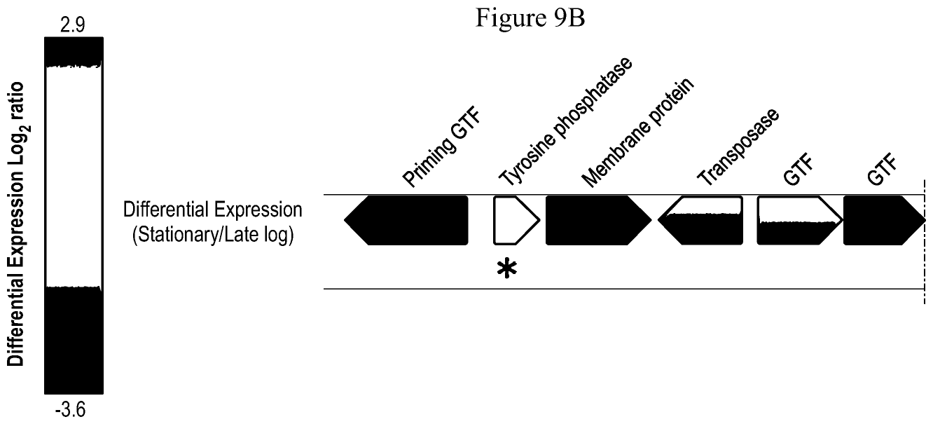

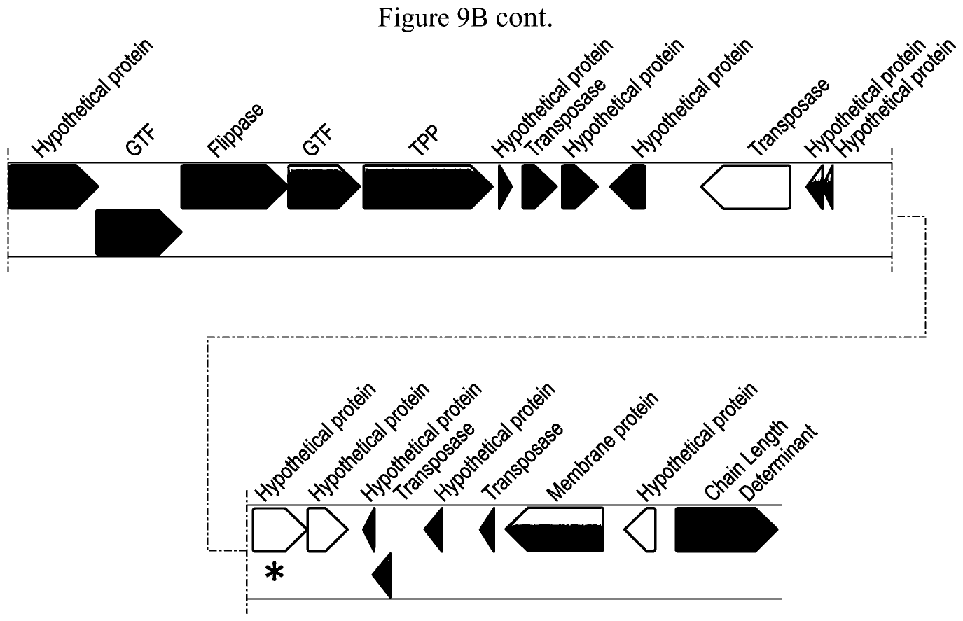

In a preferred embodiment, the bacterial strain for use in the invention both has low adherence to human intestinal epithelial cells, in particular Caco-2 cells, in YCFA compared to one or more of the B. breve strains listed in FIG. 9 (such as adherence of less than 1% of total culture, such as preferably less than 0.5% or less than 0.3%), and produces more bound surface exopolysaccharides compared to one or more of the B. breve strains listed in FIG. 9.

In certain preferred embodiments, the bacterial strain for use in the invention is able to ferment the polysaccharide raffinose, for example when cultured in an appropriate suspension medium (such as API suspension medium) at 37° C. for 4 hours.

In certain embodiments, the bacterial strain for use in the invention has reduced ability to ferment α-glucosidase and/or β-glucosidase compared to Bifidobacteria, in particular B. breve, for example when cultured in an appropriate suspension medium (such as API suspension medium) at 37° C. for 4 hours.

In certain embodiments, the bacterial strain for use in the invention comprises one or more of the genes listed in Table 1 of WO2016/203223, which is herein incorporated by reference, such as 5, 10, 20, 50 or all of the genes in Table 1 of WO2016/203223. In certain embodiments, the bacterial strain for use in the invention comprises one or more of the genes listed in Table 1 of WO2016/203223 that are highlighted with single underlining, such as Transmembrane component BL0694 of energizing module of predicted ECF transporter and/or Duplicated ATPase component BL0693 of energizing module of predicted ECF transporter. In certain embodiments, the bacterial strain for use in the invention comprises one or more of the genes listed in Table 1 of WO2016/203223 that are highlighted with double underlining and in bold, such as 1, 2, 3, 4 or 5 genes selected from: maltodextrin glucosidase (EC 3.2.1.20), putative galactosidase, cellulose synthase (UDP-forming) (EC 2.4.1.12), chitinase (EC 3.2.1.14) and sensory box/GGDEF family protein. In certain embodiments, the bacterial strain for use in the invention comprises one or more of the genes listed in Table 1 of WO2016/203223that are highlighted with italics, such as 1, 2, 3, 4, 5, 6, 7, 8 or 9 genes selected from: omega-3 polyunsaturated fatty acid synthase subunit PfaA, Type I polyketide synthase, putative glycosyl hydrolase of unknown function (DUF1680), ATPase component BioM of energizing module of biotin ECF transporter, Cation-transporting ATPase E1-E2 family, Ribose ABC transport system permease protein RbsC (TC 3.A.1.2.1), Ribose ABC transport system ATP-binding protein RbsA (TC 3.A.1.2.1), 3′-to-5′ oligoribonuclease (orn), membrane protein related to Actinobacillus protein (1944168).

In preferred embodiments, the bacterial strain for use in the invention comprises one or more (such as 5, 10, 15, 20, 25, 30, 40, 45, 50 or all) genes selected from: 2-succinyl-5-enolpyruvyl-6-hydroxy-3-cyclohexene-l-carboxylic-acid synthase (EC 2.2.1.9); 3′-to-5′ oligoribonuclease (orn); Alpha-galactosidase (EC 3.2.1.22); ATPase component of general energizing module of ECF transporters; ATPase component STY3233 of energizing module of queuosine-regulated ECF transporter; ATP-dependent DNA helicase recG (EC 3.6.1.-); Beta-glucosidase (EC 3.2.1.21); Cellulose synthase (UDP-forming) (EC 2.4.1.12); Chitinase (EC 3.2.1.14); COG1309: Transcriptional regulator; D-alanyl-D-alanine carboxypeptidase (EC 3.4.16.4); Duplicated ATPase component BL0693 of energizing module of predicted ECF transporter; Fructokinase (EC 2.7.1.4); Glucose/mannose:H+ symporter GlcP; Glycosyltransferase (EC 2.4.1.-); GMP synthase [glutamine-hydrolyzing] (EC 6.3.5.2); Hypothetical sugar kinase in cluster with indigoidine synthase indA , PfkB family of kinases; Inosine-uridine preferring nucleoside hydrolase (EC 3.2.2.1); LSU ribosomal protein L31p @ LSU ribosomal protein L31p, zinc-independent; LSU ribosomal protein L33p @ LSU ribosomal protein L33p, zinc-independent; Maltodextrin glucosidase (EC 3.2.1.20); Membrane protein, related to Actinobacillus protein (1944168); Membrane-bound lytic murein transglycosylase D precursor (EC 3.2.1.−); Methyltransferase (EC 2.1.1.−); NADH-dependent butanol dehydrogenase A (EC 1.1.1.−); Phosphoglycolate phosphatase (EC 3.1.3.18); Phosphoribosylanthranilate isomerase (EC 5.3.1.24); Putative glycosyl hydrolase of unknown function (DUF1680); Rhamnose-containing polysacharide translocation permease; Ribokinase (EC 2.7.1.15); Ribose ABC transport system, ATP-binding protein RbsA (TC 3.A.1.2.1); Ribose ABC transport system, ATP-binding protein RbsA (TC 3.A.1.2.1);

Ribose ABC transport system, high affinity permease RbsD (TC 3.A.1.2.1); Ribose ABC transport system, periplasmic ribose-binding protein RbsB (TC 3.A.1.2.1); Ribose ABC transport system, permease protein RbsC (TC 3.A.1.2.1); Ribose ABC transport system, permease protein RbsC (TC 3.A.1.2.1); Sorbitol dehydrogenase (EC 1.1.1.14); SSU ribosomal protein S14p (S29e) @ SSU ribosomal protein S14p (S29e), zinc-independent; Substrate-specific component STY3230 of queuosine-regulated ECF transporter; Sucrose-6-phosphate hydrolase (EC 3.2.1.B3); Teichoic acid export ATP-binding protein TagH (EC 3.6.3.40); Transmembrane component BL0694 of energizing module of predicted ECF transporter; Transmembrane component STY3231 of energizing module of queuosine-regulated ECF transporter; Two-component response regulator colocalized with HrtAB transporter; Type I restriction-modification system, DNA-methyltransferase subunit M (EC 2.1.1.72); Type I restriction-modification system, restriction subunit R (EC 3.1.21.3); Type I restriction-modification system, specificity subunit S (EC 3.1.21.3); Type I restriction-modification system, specificity subunit S (EC 3.1.21.3); Type I restriction-modification system, specificity subunit S (EC 3.1.21.3); Xylitol dehydrogenase (EC 1.1.1.9); and Xylose ABC transporter, periplasmic xylose-binding protein XylF. In preferred embodiments, the bacterial strain for use in the invention comprises one or more (such as 5, 10, 15, 20, 25, 30, 35 or all) genes that are listed in the preceding sentence and that are not highlighted in Table 1 of WO2016/203223.

Therapeutic Uses

Stimulating the Immune System

The examples show that administration of the compositions of the invention can lead to immune stimulation. Since administration of the compositions of the invention were shown to have an immunostimulatory effect, compositions of the invention may be useful in the treatment of disease, in particular diseases characterised by reduced immune activation and diseases treatable by an increased immune response. In certain embodiments, the compositions of the invention are for use in stimulating the immune system. In certain embodiments, the compositions of the invention are for use in treating disease by stimulating the immune system. In certain embodiments, the compositions of the invention are for use in promoting an immune response. Preferably, the invention provides a composition comprising the strain deposited under accession number 42380 at NCIMB, or a derivative or biotype thereof, for any such use.

Immunodeficiency is a state in which a patient's immune system is compromised or entirely absent. An immunodeficiency disease is an example of disease that is characterised by reduced immune activation and where it would be advantageous to stimulate the patient's immune system in order to treat the disease. In some embodiments, the compositions of the invention are for use in treating or preventing an immunodeficiency disease.

There are two types of immunodeficiency diseases. Primary immunodeficiency diseases are inherited immune disorders resulting from genetic mutations that are usually present at birth and diagnosed in childhood. Secondary immunodeficiency diseases are acquired immunodeficiencies that are the result of a disease or an environment source, such as a toxic chemical. In some embodiments, the compositions of the invention are for use in treating or preventing a primary immunodeficiency disease or a secondary immunodeficiency disease.

Examples of primary immunodeficiency disorders and examples include X-linked agammaglobulinemia (XLA), chronic granulomatous disease (CGD), common variable immunodeficiency (CVID) and severe combined immunodeficiency (SCID), which is also known as alymphocytosis or “boy in a bubble” disease. Secondary immunodeficiency disorders can be caused by for example, severe burns, chemotherapy, radiation, diabetes, malnutrition. Examples of secondary immunodeficiency disorders include AIDS, cancers of the immune system, such as leukaemia, immune-complex diseases, such as viral hepatitis multiple myeloma [27].Interferon-γ is an approved therapy for the immunodeficiency disease CGD [28]The examples demonstrate that the compositions of the invention can increase the production of IFN-y, therefore the compositions of the invention may be particularly effective at treating immunodeficiency diseases, including primary and secondary immunodeficiency diseases.

In preferred embodiments, the composition of the invention is for use in stimulating the immune system through stimulating TLR2. In certain embodiments, the composition of the invention is for stimulating TLR2 in the treatment of disease. In certain embodiments, the composition of the invention is for use in treating a disease associated with decreased TLR2 activity, or is for use in treating a patient identified as having decreased TLR2 activity. Preferably, the invention provides a composition comprising the strain deposited under accession number 42380 at NCIMB, or a derivative or biotype thereof, for any such use.

In certain embodiments, treatment with compositions of the invention drives a Th1 cell response. In certain embodiments, the compositions of the invention are for use in driving a Th1 cell response in the treatment of disease. In certain embodiments, the composition of the invention is for use in treating a disease associated with decreased Th1 cell activity, or is for use in treating a patient identified as having decreased Th1 cell activity. Preferably, the invention provides a composition comprising the strain deposited under accession number 42380 at NCIMB, or a derivative or biotype thereof, for any such use.

In preferred embodiments, the composition of the invention is for use in stimulating the immune system through activating NFκB. In certain embodiments, the composition of the invention is for stimulating NFκB in the treatment of disease. In certain embodiments, the composition of the invention is for use in treating a disease associated with decreased NFκB activity, or is for use in treating a patient identified as having decreased NFκB activity. Preferably, the invention provides a composition comprising the strain deposited under accession number 42380 at NCIMB, or a derivative or biotype thereof, for any such use.

Compositions of the invention may be useful in the treatment of diseases characterised by decreased levels of activated CD8⁺ cells. In one embodiment, compositions of the invention are for use in stimulating the immune response by increasing the activity or levels of CD8⁺ cells. In one embodiment, composition of the invention are for use in treating disease by increasing the activity or levels of CD8³⁰ cells. In one embodiment, compositions of the invention are for use in stimulating the immune response by activating CD8⁺ cells.

Compositions of the invention may be useful in the treatment of diseases characterised by decreased levels of B cells. In one embodiment, compositions of the invention are for use in stimulating the immune response by increasing the activity or levels of B cells. In one embodiment, composition of the invention are for use in treating disease by increasing the activity or levels of B cells. In one embodiment, compositions of the invention are for use in stimulating the immune response by activating B cells.

Compositions of the invention may be useful in the treatment of diseases characterised by decreased levels of activated CD8⁺CD25⁺ cells. In one embodiment, compositions of the invention are for use in stimulating the immune response by increasing the activity or levels of CD8⁺CD25⁺ cells. In one embodiment, composition of the invention are for use in treating disease by increasing the activity or levels of CD8⁺CD25⁺ cells. In one embodiment, compositions of the invention are for use in stimulating the immune response by activating CD8⁺CD25⁺ cells.

Compositions of the invention may be useful in the treatment of diseases characterised by a decrease in the number or percentage of B cells. In one embodiment, the compositions of the invention are for use in treating or preventing diseases characterised by decrease in the number or percentage of B cells. In one embodiment, the compositions of the invention are for use in treating or preventing diseases by increasing the number or percentage of B cells in cell populations, wherein the increase in number or percentage of B cells results in immune stimulation. In one embodiment, compositions of the invention are for use in stimulating the immune response by increasing the number or percentage of B cells.

The examples show that administration of the compositions of the invention can lead to an increase in expression of pro-inflammatory molecules, such as pro-inflammatory cytokines. Examples of pro-inflammatory molecules that showed an increase in expression levels upon administration of compositions of the invention include IL-12p70, TNF-α, IL-4, IFNγ and IL-17α. Since administration of the compositions of the invention were shown to increase the expression of pro-inflammatory molecules, compositions of the invention may be useful in the treatment of diseases characterised by a decrease in expression of pro-inflammatory molecules, such as pro-inflammatory cytokines. In one embodiment, the compositions of the invention are for use in treating or preventing diseases characterised by a decrease in the expression and/or activity of pro-inflammatory molecules, in particular diseases characterised by a decrease in the expression and/or activity of pro-inflammatory cytokines. In a particular embodiment, the compositions of the invention are for use in treating or preventing diseases characterised by a decrease in the expression and/or activity of IL-12p70, TNF-α, IL-4 and/or IFNγ. In one embodiment, the compositions of the invention are for use in treating or preventing diseases by increasing the expression and/or activity of IL-12p70, TNF-α, IL-4 and/or IFNγ. In one embodiment, compositions of the invention are for use in promoting the immune response by increasing the expression and/or activity of IL-12p70, TNF-α, IL-4 and/or IFNγ. In a particular embodiment, the compositions of the invention are for use in treating or preventing diseases characterised by a decrease in the expression and/or activity of IL-17α IL-12p70, TNF-α, IL-4, IFNγ and/or IL-17α. In one embodiment, the compositions of the invention are for use in treating or preventing diseases by increasing the expression and/or activity of IL-12p70, TNF-α, IL-4 IFNγ and/or IL-17α. In one embodiment, compositions of the invention are for use in promoting the immune response by increasing the expression and/or activity of IL-12p70, TNF-α, IL-4 IFNγ and/or IL-17α.

The examples also show that administration of the compositions of the invention can lead to an increase in expression of IL-1β. IL-1β is a pro-inflammatory cytokine [29]. The production and secretion of IL-1β is regulated by the inflammasome, a protein complex which is associated with activation of the inflammatory response [30]. Since administration of the compositions of the invention were shown to increase the expression of IL-1β, compositions of the invention may be useful in the treatment of diseases characterised by a decrease in expression of IL-1β. In a particular embodiment, the compositions of the invention are for use in treating or preventing diseases characterised by a decrease in the expression and/or activity of IL-1β. In one embodiment, the compositions of the invention are for use in treating or preventing diseases by increasing the expression and/or activity of IL-1β.

The examples show that administration of the compositions of the invention can lead to an increase in expression of Tumour Necrosis Factor alpha (TNF-α). TNF-α is a pro-inflammatory cytokine which is known to be involved in various signalling pathways to promote cell death. TNF-α initiates apoptosis by binding to its cognate receptor, TNFR-1, which leads to a cascade of cleavage events in the apoptotic pathway [31]. TNF-α can also trigger necrosis via a RIP kinase-dependent mechanism [32]. Since administration of the compositions of the invention show an increase in TNF-α expression, compositions of the invention may be useful in the treatment of diseases, in particular for use in treating or preventing diseases characterised by a decrease in expression of by TNF-α. In one embodiment, the compositions of the invention are for use in treating diseases characterised by decreased TNF-α expression. In a particular embodiment, the compositions of the invention are for use in treating or preventing diseases characterised by a decrease in the expression and/or activity of TNF-α. In one embodiment, the compositions of the invention may be useful for treating or preventing diseases by increasing the expression and/or activity of TNF-α. In one embodiment, compositions of the invention are for use in promoting the immune response by increasing the expression and/or activity of TNF-α.

Since administration of the compositions of the invention show an increase in IL-4 expression, compositions of the invention may be useful in the treatment of diseases, in particular for use in treating or preventing diseases characterised by a decrease in expression of by IL-4. In one embodiment, the compositions of the invention are for use in treating diseases characterised by decreased IL-4 expression. In a particular embodiment, the compositions of the invention are for use in treating or preventing diseases characterised by a decrease in the expression and/or activity of IL-4. In one embodiment, the compositions of the invention may be useful for treating or preventing diseases by increasing the expression and/or activity of IL-4. In one embodiment, compositions of the invention are for use in promoting the immune response by increasing the expression and/or activity of IL-4.

Since administration of the compositions of the invention show an increase in IL-17α expression, compositions of the invention may be useful in the treatment of diseases, in particular for use in treating or preventing diseases characterised by a decrease in expression of by IL-17α. In one embodiment, the compositions of the invention are for use in treating diseases characterised by decreased IL-17α expression. In a particular embodiment, the compositions of the invention are for use in treating or preventing diseases characterised by a decrease in the expression and/or activity of IL-17α. In one embodiment, the compositions of the invention may be useful for treating or preventing diseases by increasing the expression and/or activity of IL-17α. In one embodiment, compositions of the invention are for use in promoting the immune response by increasing the expression and/or activity of IL-17α.

Since administration of the compositions of the invention show an increase in IL-12p70 expression, compositions of the invention may be useful in the treatment of diseases, in particular for use in treating or preventing diseases characterised by a decrease in expression of by IL-12p70. In one embodiment, the compositions of the invention are for use in treating diseases characterised by decreased IL-12p70 expression. In a particular embodiment, the compositions of the invention are for use in treating or preventing diseases characterised by a decrease in the expression and/or activity of IL-12p70. In one embodiment, the compositions of the invention may be useful for treating or preventing diseases by increasing the expression and/or activity of IL-12p70. In one embodiment, compositions of the invention are for use in promoting the immune response by increasing the expression and/or activity of IL-12p70.

In certain embodiments, the disease to be treated by the compositions of the invention is not cancer.

In certain embodiments, the disease to be treated by the composition of the invention is not mediated by IL-17 or the Th17 pathway. In certain embodiments, the compositions of the invention increase expression or activity of IL-17 and/or the Th17 pathway.

In preferred embodiments of the invention, the subject to whom the composition of the invention is administered is not taking linoleic acid supplements and/or does not have a diet rich in linoleic acid. Additionally or alternatively, the composition of the invention does not comprise linoleic acid.

In embodiments, the composition of the invention does not comprise betα-galacto-oligosaccharides A and/or B.

Patients in need of immune stimulation may be at risk of bacterial infections. The examples demonstrate that the compositions of the invention have anti-microbial activity. Therefore, in certain embodiments, the compositions of the invention are for use in stimulating the immune system and treating or preventing a bacterial infection. In certain embodiments, the compositions of the invention are for use in treating a bacterial infection by stimulating the immune system and inhibiting growth of the bacterial infection. In certain embodiments, the compositions of the invention are for use in promoting an immune response against a pathogenic bacteria and inhibiting growth of the bacteria. Preferably, the bacterial infection is of the gastrointestinal tract. Preferably the bacterial infection is of Gram-negative bacteria.

The examples also demonstrate that the compositions of the invention promote the differentiation of T-helper cells and cytotoxic T lymphocytes. Therefore, in certain embodiments, the compositions of the invention are for use in stimulating the differentiation of T-helper cells and/or cytotoxic T lymphocytes.

Use as a Vaccine Adjuvant

The examples show that administration of the compositions of the invention stimulate the immune system and can lead to an increase in expression of Tumour Necrosis Factor alpha (TNF-α) and activation of TLR2. TNF-α is known to be important for vaccine responses. For example, TNF-α has been shown to be required for an efficient vaccine response in a flu vaccination of the elderly population [33]. Similarly, TLR2 is an important target for vaccine adjuvants to improve responses [34]. Since administration of the compositions of the invention were shown to increase TNF-a expression and TLR2 activity, compositions of the invention may be useful as a vaccine adjuvant. In one embodiment, the compositions of the invention are for use as a vaccine adjuvant by increasing the level and/or activity of TNF-α. In one embodiment, the compositions of the invention are for use as a vaccine adjuvant by increasing the level and/or activity of TLR2. In one embodiment, the compositions of the invention are for use as a vaccine adjuvant. In one embodiment, the compositions of the invention are for use as a vaccine adjuvant in influenza therapy. In certain embodiments, the compositions of the invention are for use in enhancing an immune response against an antigen. In certain embodiments, the invention provides a composition to be administered in combination with an antigen. In certain embodiments, the compositions of the invention are for administration to a patient shortly prior to or after vaccination. Preferably, the invention provides a composition comprising the strain deposited under accession number 42380 at NCIMB, or a derivative or biotype thereof, for any such use as a vaccine adjuvant.

The examples demonstrate that the bacteria of the invention activate TLR2. TLR agonists are in development as vaccine adjuvants across a range of antigen types, particularly in the elderly population [35]. Therefore, the compositions of the invention may be useful as vaccine adjuvants, in particular for vaccine administered to elderly patients (e.g. over 40, 50, 60, 70 or 80 years of age), who may have reduced immune system activity. TLR2 signalling also plays a key role in age-associated innate immune responses [36]. In certain embodiments, the compositions are for use in enhancing an innate immune response. Although TLR2 agonists are in development as vaccine adjuvants, these are all from known pathogens and/or synthetic. In contrast, the compositions of the invention comprise commensal bacteria.

The examples also show that administration of the compositions of the invention can lead to an increase in expression of IL-1β. Li et al. [37] showed that the adjuvant aluminium hydroxide activated the secretion of IL-1β, and suggested that IL-1β itself can act as an adjuvant. Since administration of the compositions of the invention were shown to increase IL-1β expression, compositions of the invention may be useful as a vaccine adjuvant.

The examples demonstrate that the compositions of the invention can increase IFNγ levels and promote a Th1 cell response, both of which are associated with increase antibody responses against antigens [38]. In certain embodiments, the compositions of the invention are for use in promoting an antibody response against an antigen, in particular a pathogenic or cancer antigen. Also, is an IFN-γ measure of vaccine induced T-cell responses in volunteers receiving investigated malaria vaccines [39]. In certain embodiments, the compositions of the invention are for use in promoting a T-cell response against an antigen, in particular a pathogenic or cancer antigen. In one embodiment, the compositions of the invention are for use as a vaccine adjuvant by increasing the level and/or activity of IFN-γ. In certain embodiments, the compositions are for use in protecting against malaria.

The examples also show that administration of the compositions of the invention can lead to an increase in expression or levels of IL-12p70. This effect has been associated with vaccine adjuvant efficiency and IL-12 has been proposed as an adjuvant itself [40], which suggests the compositions of the invention will be effective as adjuvants. In one embodiment, the compositions of the invention are for use as a vaccine adjuvant by increasing the level and/or activity of IL-12p70.

In some embodiments, when used as a vaccine adjuvant, the compositions of the invention will be administered on their own to provide an adjuvant effect for an antigen that has been separately administered to the patient. In certain embodiments, the composition of the invention is administered orally, whilst the antigen is injected parenterally.

The compositions of the invention may be used for enhancing an immune response to any useful antigen. Exemplary antigens for use with the invention include: viral antigens, such as viral surface proteins; bacterial antigens, such as protein and/or saccharide antigens; fungal antigens; parasite antigens; and tumor antigens. The invention is particularly useful for vaccines against influenza virus, HIV, hookworm, hepatitis B virus, herpes simplex virus, rabies, respiratory syncytial virus, cytomegalovirus, Staphylococcus aureus, chlamydia, SARS coronavirus, varicella zoster virus, Streptococcus pneumoniae, Neisseria meningitidis, Mycobacterium tuberculosis, Bacillus anthracis, Epstein Barr virus, human papillomavirus, etc. Further antigens for use with the invention include glycoprotein and lipoglycan antigens, archaea antigens, melanoma antigen E (MAGE), Carcinoembryonic antigen (CEA), MUC-1, HER2, sialyl-Tn (STn), human telomerase reverse transcriptase (hTERT), Wilms tumour gene (WT1), CA-125, prostate-specific antigen (PSA), Epstein-Barr virus antigens, neoantigens, oncoproteins, amyloid-beta, Tau, PCSK9 and habit forming substances, for example nicotine, alcohol or opiates.

Preferred antigens for use with the invention include pathogen antigens and tumour antigens. An antigen will elicit an immune response specific for the antigen that will be effective for protecting against infection with the pathogen or attacking the tumour. Antigens may be, for example, peptides or polysaccharides.

The invention also provides the use of: (i) an aqueous preparation of an antigen; and (ii) a composition comprising a bacterial strain of the species B. breve, in the manufacture of a medicament for raising an immune response in a patient. Preferably, the bacterial strain is the strain deposited under accession number 42380 at NCIMB, or a derivative or biotype thereof.

The immune response raised by these methods and uses will generally include an antibody response, preferably a protective antibody response.

In some embodiments, a bacterial strain of the species Bifidobacterium breve is engineered to present an antigen. Presenting an antigen on the bacterial strain of the invention may maximise the immunostimulatory activities and further enhance the protective immune response generated against the antigen. In addition, manufacturing and delivering therapeutics comprising an antigen and a bacteria of the invention may be more efficient and effective this way than when each of the antigen and the composition comprising the bacterial strain are manufactured and administered separately. Therefore, in some embodiments, the invention provides a composition comprising a bacterial strain of the species Bifidobacterium breve that presents an antigen, for example on its cell surface. In some embodiments, the composition comprising the bacterial strain that presents an antigen is for use as a vaccine antigen. In some embodiments, the antigen is derived from HIV, hookworm, hepatitis B virus, herpes simplex virus, rabies, respiratory syncytial virus, cytomegalovirus, Staphylococcus aureus, chlamydia, SARS coronavirus, varicella zoster virus, Streptococcus pneumoniae, Neisseria meningitidis, Mycobacterium tuberculosis, Bacillus anthracis, Epstein Barr virus or human papillomavirus. In some embodiments, the antigen is a glycoprotein antigen, lipoglycan antigen, archaea antigen, melanoma antigen E (MAGE), Carcinoembryonic antigen (CEA), MUC-1, HER2, sialyl-Tn (STn), human telomerase reverse transcriptase (hTERT), Wilms tumour gene (WT1), CA-125, prostate-specific antigen (PSA), Epstein-Barr virus antigens, neoantigens, oncoproteins, amyloid-beta, Tau, PCSK9 or a habit forming substance, such as, alcohol, opiates and the like.

In some embodiments, the bacteria of the invention expresses one or more antigens. Generally the antigen will be expressed recombinantly and will be heterologous to the bacteria of the invention. Therefore, the invention provides a bacterial strain of the species Bifidobacterium breve that expresses a heterologous antigen. The antigen may be part of a fusion polypeptide expressed with one or more polypeptides homologous to the bacteria. In some embodiments, the bacteria expresses the antigen as a non-fusion polypeptide. In some embodiments, the invention provides a composition comprising a cell of a bacterial strain of the species Bifidobacterium breve, wherein the cell expresses a heterologous antigen. In some embodiments, the composition is for use as a vaccine. In some embodiments, the invention provides a cell of a bacterial strain of the species Bifidobacterium breve, wherein the cell expresses a heterologous antigen. In some embodiments, the cell is for use as a vaccine.

Exemplary antigens for use with the invention include: viral antigens, such as viral surface proteins; bacterial antigens, such as protein and/or saccharide antigens; fungal antigens; parasite antigens; and tumor antigens. Further antigens for expressing in a bacterial strain of the species Bifidobacterium breve include glycoprotein and lipoglycan antigens, archaea antigens, melanoma antigen E (MAGE), Carcinoembryonic antigen (CEA), MUC-1, HER2, sialyl-Tn (STn), human telomerase reverse transcriptase (hTERT), Wilms tumour gene (WT1), CA-125, prostate-specific antigen (PSA), Epstein-Barr virus antigens, neoantigens, oncoproteins, amyloid-beta, Tau, PCSK9 and habit forming substances, for example nicotine, alcohol, opiates, or the like.

The invention may also be useful for enhancing the response to vaccines against non-communicable diseases such as Alzheimer's Disease and other neurodegenerative disorders, in which case the antigen for use with the invention may be amyloid-beta or Tau. Other such antigens for non-communicable diseases include PCSK9 (for the treatment of elevated cholesterol).

The invention may also be useful for enhancing the response to vaccines against habit forming substances, for example nicotine, alcohol or opiates.

The examples also demonstrate that the compositions of the invention have anti-microbial activity. Therefore, the compositions of the invention may be particularly effective for use in vaccines against bacterial infections, in particular Gram-negative bacterial infections. The compositions of the invention may exert an anti-microbial effect against the bacterial infection whilst also stimulating the immune system to tackle the infection. Therefore, in certain embodiments, the compositions of the invention are treating a bacterial infection and preventing future bacterial infections.

Cell Therapies

Chimeric Antigen Receptor T cell (CAR-T) Therapy

The examples also show that administration of the compositions of the invention can lead to an increase in activation of TLR2. TLR2 stimulation potentiates the efficacy of CAR-T therapy [41]. Therefore, compositions of the invention may be useful in cell therapy, in particular CAR-T cell therapy. In one embodiment, the compositions of the invention are for use in cell therapy. In one embodiment, the compositions of the invention are for use in CAR-T cell therapy. In one embodiment, compositions of the invention are for use in the treatment of chronic lymphocyte leukaemia. Preferably, the invention provides a composition comprising the strain deposited under accession number 42380 at NCIMB, or a derivative or biotype thereof, for any such use.

The examples also show that administration of the compositions of the invention can lead to an increase in activation of NFκB. NFκB activation improves the potency of CAR-T therapy [42]. Therefore, compositions of the invention may be useful in cell therapy, in particular CAR-T cell therapy.

In certain embodiments, the compositions of the invention are administered to a patient before T cell adoptive transfer during CAR-T therapy.

In certain embodiments, the compositions of the invention are administered to a patient after T cell adoptive transfer during CAR-T therapy.

Therefore, the compositions of the invention may be useful in cell therapy, in particular in enhancing the response to a cell therapy.

Mesynchymal Stem Cell (MSC) therapy

Mesynchymal stem cell (MSC) therapy has been reported to have immunostimulatory properties. When MSCs are treated with LPS, they upregulate pro-inflammatory cytokines IL-8 which causes increased B cell proliferation [43]. Therefore, since compositions of the invention were shown to increase the expression of B cell proliferation, they may be useful in combination with MSC cell therapy.

Stem Cell Transplantation Therapy

It has been reported that, instead of using undifferentiated stem cells in stem cell transplantation therapy, it may be beneficial to differentiate stem cells to some extent prior to transplantation. For example, Heng et al. [44] reported that cardiomyogenic differentiation of stem cells may be beneficial by having a higher engraftment efficiency, enhanced regeneration of myocytes and increased restoration of heart function. Also, studies have shown that GI colonisation with certain commensal strains of bacteria can improve survival following allogeneic haematopoietic cell transplant [45]. Since administration of the compositions of the invention stimulated cells, compositions of the invention may be useful for stem cell differentiation in stem cell transplantation therapy.

Immunosenescence

Fulop et al. [46] identified that an increase in Treg cell number and a decrease in B cell number are associated with aging in the adaptive immune system. Therefore, compositions of the invention may be used to prevent or delay immunosenescence. In one embodiment, compositions of the invention are for use in preventing immunosenescence. In another embodiment, compositions of the invention are for use in delaying immunosenescence characterised by an increase in Treg cell number. In another embodiment, compositions of the invention are for use in delaying immunosenescence characterised by a decrease in B cell number. In another embodiment, compositions of the invention are for use in delaying immunosenescence characterised by an increase in Treg cell number and a decrease in B cell number. In one embodiment, compositions of the invention are for use in delaying immunosenescence by decreasing Treg cell number. In one embodiment, compositions of the invention are for use in delaying immunosenescence by increasing B cell number. In another embodiment, compositions of the invention are for use in delaying immunosenescence by decreasing Treg cell number and increasing B cell number. In one embodiment, compositions of the invention are for use in treating diseases caused by immunosenescence. In one embodiment, compositions of the invention are for use in treating aging-related diseases by delaying and/or preventing immunosenescence. Preferably, the invention provides a composition comprising the strain deposited under accession number 42380 at NCIMB, or a derivative or biotype thereof, for any such use.

Furthermore, it has been proposed that vaccine adjuvants may overcome immunosenescence [47]. Since the compositions of the invention are suitable for use as a vaccine adjuvant, compositions of the invention may be useful for preventing or delaying immunosenescence. In another embodiment, compositions of the invention are for use in delaying and/or preventing immunosenescence as a vaccine adjuvant. In another embodiment, compositions of the invention are for use as a vaccine adjuvant, wherein the compositions delay and/or prevent immunosenescence.

Diseases that are associated with immunosenescence include cardiovascular disease, neurodegenerative diseases, such as Alzheimer's disease and Parkinson's disease, cancer, diabetes mellitus type 2 [48] and autoimmune disorders [49].

Subjects suffering from immunosenescence may be susceptible to bacterial infections. The examples show that the compositions of the invention have anti-microbial activity. Therefore, in certain embodiments, the compositions of the invention are for use in treating or preventing a bacterial infection in a patient exhibiting immunosenescence, such as an elderly patient, or a patient over 50, 55, 60, 65, 70 or 75 years of age.

Treating and Preventing Bacterial Infections

The examples demonstrate that B. breve and particularly the B. breve strains of the invention have potent anti-microbial activity. Therefore, in certain embodiments, the compositions of the invention are for treating or preventing a bacterial infection.

In preferred embodiments, the infection is a gastro-intestinal infection. B. breve strains, and in particular B. breve strains of the invention have been shown to have potent effects when administered to the gastro-intestinal tract (see the examples and WO2016/2032). In preferred embodiments, the infection is a Gram-negative bacterial infection. In especially preferred embodiments, the infection is a Gram-negative gastro-intestinal infection, such as a Helicobacter pylori, Salmonella enteritidis, Salmonella typhi or E. coli infection. Generally, the bacterial infection is a pathogenic bacterial infection. In preferred embodiments, the composition of the invention is for use in treating or preventing gastrointestinal E. coli infection. In further preferred embodiments, the composition of the invention is for use in treating or preventing gastrointestinal S. enterica infection.

In some embodiments, the bacterial infection is of a genus selected from the list consisting of: Escherichia, Klebsiella, Salmonella, and Bacillus. In some embodiments, the bacterial infection for treatment or prevention is E. coli infection. In some embodiments, the bacterial infection for treatment or prevention is Klebsiella pneumoniae infection. In some embodiments, the bacterial infection for treatment or prevention is S. Typhimurium infection. In some embodiments, the bacterial infection for treatment or prevention is B. subtilis infection. The compositions of the invention are shown to have potent anti-microbial activity against these bacteria.

In some embodiments, the bacterial infection for treatment or prevention is Pseudomonas aeruginosa infection. In some embodiments, the bacterial infection for treatment or prevention is Neisseria gonorrhoeae infection. In some embodiments, the bacterial infection for treatment or prevention is, Chlamydia trachomatis infection. In some embodiments, the bacterial infection for treatment or prevention is Yersinia pestis infection. In some embodiments, the bacterial infection for treatment or prevention is Neisseria meningitidis infection. In some embodiments, the bacterial infection for treatment or prevention is Moraxella catarrhalis infection. In some embodiments, the bacterial infection for treatment or prevention is Haemophilus influenzae infection. In some embodiments, the bacterial infection for treatment or prevention is Legionella pneumophila infection. In some embodiments, the bacterial infection for treatment or prevention is Pseudomonas aeruginosa, infection. In some embodiments, the bacterial infection for treatment or prevention is Proteus mirabilis infection. In some embodiments, the bacterial infection for treatment or prevention is Enterobacter cloacae infection. In some embodiments, the bacterial infection for treatment or prevention is Serratia marcescens infection. In some embodiments, the bacterial infection for treatment or prevention is Helicobacter pylori infection. In some embodiments, the bacterial infection for treatment or prevention is Salmonella Enteritidis infection. In some embodiments, the bacterial infection for treatment or prevention is Salmonella Typhi infection. In some embodiments, the bacterial infection for treatment or prevention is Salmonella Paratyphi infection. These bacteria are Gram-negative so may be susceptible to the compositions of the invention, as shown in the examples.

In some embodiments, the composition of the invention is for use in reducing the viability of a bacteria in the treatment of a bacterial infection. The bacteria of the invention may be used to restore the level of pathogenic bacteria to asymptomatic levels or to eliminate the pathogenic bacteria entirely from a subject, thereby treating the bacterial infection, in addition to alleviating the symptoms associated with the elevated level of the bacteria. In some embodiments, the composition of the invention is for use in inhibiting the growth of a bacteria in the treatment of prevention of a bacterial infection. In other words, the composition may have cytostatic activity with respect to bacteria causing an infection.

In certain embodiments, the composition delays the onset of a recurrent infection or prevents a recurrent infection. In certain embodiments, the subject to be treated is at risk of developing a bacterial infection, such as is an asymptomatic carrier of the bacteria. In other embodiments, the compositions of the invention are for use in treating a patient exhibiting symptoms of a bacterial infection.

Preferably, the bacteria used in the invention for treating or preventing a bacterial infection is able to ferment raffinose, for example when cultured in an appropriate suspension medium (such as API suspension medium) at 37° C. for 4 hours.

Preferably, the bacteria used in the invention for treating or preventing a bacterial infection exhibit intermediate fermentation of β-glucosidase or intermediate fermentation of α-arabinose, or more preferably intermediate fermentation of β-glucosidase and intermediate fermentation of α-arabinose, for example when cultured in an appropriate suspension medium (such as API suspension medium) at 37° C. for 4 hours, and for example when subjected to the Rapid ID 32A analysis. The examples demonstrate that both B. breve strains MRX004 and Test 3 have useful activity and both exhibit intermediate fermentation of β-glucosidase and α-arabinose.

Preferably, the bacteria used in the invention for treating or preventing a bacterial infection do not exhibit positive fermentation of N-acetyl-β-glucosaminidase, for example when cultured in an appropriate suspension medium (such as API suspension medium) at 37° C. for 4 hours, and for example when subjected to the Rapid ID 32A analysis. The bacteria may exhibit only intermediate or no fermentation of N-acetyl-β-glucosaminidase. The examples demonstrate that both B. breve strains MRX004 and Test 2 have useful activity and neither exhibits positive fermentation of N-acetyl-β-glucosaminidase.

Preferably, the bacteria used in the invention for treating or preventing a bacterial infection exhibit intermediate fermentation of α-galactosidase or intermediate fermentation of α-arabinose, or more preferably intermediate fermentation of α-galactosidase and intermediate fermentation of α-arabinose, for example when cultured in an appropriate suspension medium (such as API suspension medium) at 37° C. for 4 hours, and for example when subjected to the Rapid ID 32A analysis. The examples demonstrate that B. breve strains MRX004 and Test 8 both have useful activity and both exhibit intermediate fermentation of α-galactosidase and α-arabinose.

In alternative embodiments, the bacteria used in the invention for treating or preventing a bacterial infection ferment serine arylamidase but not leucyl glycine arylamidase and not alanine arylamidase, for example when cultured in an appropriate suspension medium (such as API suspension medium) at 37° C. for 4 hours, and for example when subjected to the Rapid ID 32A analysis. The examples demonstrate that B. breve strains Test 11 and Test 12 both have useful activity and both ferment serine arylamidase but not leucyl glycine arylamidase and not alanine arylamidase.

In alternative embodiments, the bacteria used in the invention for treating or preventing a bacterial infection exhibit intermediate fermentation of serine arylamidase, for example when cultured in an appropriate suspension medium (such as API suspension medium) at 37° C. for 4 hours, and for example when subjected to the Rapid ID 32A analysis. The examples demonstrate that B. breve strains Test 3 and Test 7 both have potent anti-microbial activity and both exhibit intermediate fermentation of serine arylamidase.

Any suitable assay known in the art may be used to assess the ability of a bacterium to ferment a carbohydrate source or amino acid. Preferebly, the Rapid ID 32A analysis is used (preferably using the Rapid ID 32A system from bioMérieux).

Modes of Administration Preferably, the compositions of the invention are formulated to be administered to the gastrointestinal tract in order to enable delivery to and/or partial or total colonisation of the intestine with the bacterial strain of the invention. In some embodiments, the term “total colonisation of the intestine” means that bacteria have colonised all parts of the intestine (i.e. the small intestine, large intestine and rectum). In further embodiments of the invention, the term “total colonisation” or “partial colonisation” means that the bacteria are retained permanently or temporarily in the intestine, respectively. Generally, the compositions of the invention are administered orally, but they may be administered rectally, intranasally, or via buccal or sublingual routes.

In certain embodiments, the compositions of the invention may be administered as a foam, as a spray or a gel.

In certain embodiments, the compositions of the invention may be administered as a suppository, such as a rectal suppository, for example in the form of a theobroma oil (cocoa butter), synthetic hard fat (e.g. suppocire, witepsol), glycero-gelatin, polyethylene glycol, or soap glycerin composition.

In certain embodiments, the composition of the invention is administered to the gastrointestinal tract via a tube, such as a nasogastric tube, orogastric tube, gastric tube, jejunostomy tube (J tube), percutaneous endoscopic gastrostomy (PEG), or a port, such as a chest wall port that provides access to the stomach, jejunum and other suitable access ports.

The compositions of the invention may be administered once, or they may be administered sequentially as part of a treatment regimen. In certain embodiments, the compositions of the invention are to be administered daily (either once or several times).

In certain embodiments of the invention, treatment according to the invention is accompanied by assessment of the patient's gut microbiota. Treatment may be repeated if delivery of and/or partial or total colonisation with the strain of the invention is not achieved such that efficacy is not observed, or treatment may be ceased if delivery and/or partial or total colonisation is successful and efficacy is observed.

In certain embodiments, the composition of the invention may be administered to a pregnant animal, for example a mammal such as a human in order to reduce the likelihood of disease developing in her child in utero and/or after it is born.

The compositions of the invention may be administered to a patient that has been diagnosed with a disease or condition mediated reduced immune activity, or that has been identified as being at risk of a disease or condition mediated by reduced immune activity. The compositions may also be administered as a prophylactic measure to prevent the development of diseases or conditions mediated by reduced immune activity in a healthy patient.

The compositions of the invention may be administered to a patient that has been diagnosed with deficient immune activity, or that has been identified as being at risk of deficient immune activity. For example, the patient may have reduced or absent colonisation by B. breve.

The compositions of the invention may be administered as a food product, such as a nutritional supplement.

Generally, the compositions of the invention are for the treatment of humans, although they may be used to treat animals including monogastric mammals such as poultry, pigs, cats, dogs, horses or rabbits. The compositions of the invention may be useful for enhancing the growth and performance of animals. If administered to animals, oral gavage may be used.

Compositions

Generally, the composition of the invention comprises bacteria. In preferred embodiments of the invention, the composition is formulated in freeze-dried form. For example, the composition of the invention may comprise granules or gelatin capsules, for example hard gelatin capsules, comprising a bacterial strain of the invention.

Preferably, the composition of the invention comprises lyophilised bacteria. Lyophilisation of bacteria is a well-established procedure and relevant guidance is available in, for example, references [50,52]. The examples demonstrate that lyophilised compositions are particularly effective.

Alternatively, the composition of the invention may comprise a live, active bacterial culture.

In preferred embodiments, the composition of the invention is encapsulated to enable delivery of the bacterial strain to the intestine. Encapsulation protects the composition from degradation until delivery at the target location through, for example, rupturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule. Guidance on encapsulation that may be useful for preparing compositions of the invention is available in, for example, references [53] and [54].

The composition may be administered orally and may be in the form of a tablet, capsule or powder. Encapsulated products are preferred because B. breve are anaerobes. Other ingredients (such as vitamin C, for example), may be included as oxygen scavengers and prebiotic substrates to improve the delivery and/or partial or total colonisation and survival in vivo. Alternatively, the probiotic composition of the invention may be administered orally as a food or nutritional product, such as milk or whey based fermented dairy product, or as a pharmaceutical product.

In some embodiments, the composition does not comprise hydrolysed cow's whey.

A composition of the invention includes a therapeutically effective amount of a bacterial strain of the invention. A therapeutically effective amount of a bacterial strain is sufficient to exert a beneficial effect upon a patient. A therapeutically effective amount of a bacterial strain may be sufficient to result in delivery to and/or partial or total colonisation of the patient's intestine.

A suitable daily dose of the bacteria, for example for an adult human, may be from about 1×10³ to about 1×10¹¹ colony forming units (CFU); for example, from about 1×10⁷ to about 1×10¹⁰ CFU; in another example from about 1×10⁶ to about 1×10¹⁰ CFU; in another example from about 1×10⁷ to about 1×10¹¹ CFU; in another example from about 1×10⁸ to about 1×10¹⁰ CFU; in another example from about 1×10⁸ to about 1×10¹¹ CFU.

In certain embodiments, the dose of the bacteria is at least 10⁹ cells per day, such as at least 10¹⁰, at least 10¹¹, or at least 10¹² cells per day.

In certain embodiments, the composition contains the bacterial strain in an amount of from about 1×10⁶to about 1×10¹¹ CFU/g, respect to the weight of the composition; for example, from about 1×10⁸ to about 1×10¹⁰ CFU/g. The dose may be, for example, 1 g, 3 g, 5 g, and 10 g.

A dose of the composition may comprise the bacterial strain from about 1×10⁶ to about 1×10¹¹ colony forming units (CFU)/g, respect to the weight of the composition. The dose may be suitable for an adult human. For example, the composition may comprise the bacterial strain from about 1×10³ to about 1×10¹¹CFU/g; for example, from about 1×10⁷ to about 1×10¹⁰ CFU/g; in another example from about 1×10⁶ to about 1×10¹⁰ CFU/g; in another example from about 1×10⁷ to about 1×10¹¹ CFU/g; in another example from about 1×10⁸ to about 1×10¹⁰ CFU/g; in another example from about 1×10⁸ to about 1×10¹¹ CFU/g, The dose may be, for example, 1 g, 3 g, 5 g, and 10 g.

In certain embodiments, the invention provides the above pharmaceutical composition, wherein the amount of the bacterial strain is from about 1×10³ to about 1×10¹¹ colony forming units per gram with respect to a weight of the composition.

In certain embodiments, the invention provides the above pharmaceutical composition, wherein the composition is administered at a dose of between 500 mg and 1000 mg, between 600 mg and 900 mg, between 700 mg and 800 mg, between 500 mg and 750 mg or between 750 mg and 1000 mg. In certain embodiments, the invention provides the above pharmaceutical composition, wherein the lyophilised bacteria in the pharmaceutical composition is administered at a dose of between 500 mg and 1000 mg, between 600 mg and 900 mg, between 700 mg and 800 mg, between 500 mg and 750 mg or between 750 mg and 1000 mg.

The composition may be formulated as a probiotic. A probiotic is defined by the FAO/WHO as a live microorganism that, when administered in adequate amounts, confers a health benefit on the host.

Typically, a probiotic, such as the composition of the invention, is optionally combined with at least one suitable prebiotic compound. A prebiotic compound is usually a non-digestible carbohydrate such as an oligo- or polysaccharide, or a sugar alcohol, which is not degraded or absorbed in the upper digestive tract. Known prebiotics include commercial products such as inulin and transgalacto-oligosaccharides.

In certain embodiments, the probiotic composition of the present invention includes a prebiotic compound in an amount of from about 1 to about 30% by weight, respect to the total weight composition, (e.g. from 5 to 20% by weight). Carbohydrates may be selected from the group consisting of: fructo-oligosaccharides (or FOS), short-chain fructo-oligosaccharides, inulin, isomalt-oligosaccharides, pectins, xylo-oligosaccharides (or XOS), chitosan-oligosaccharides (or COS), beta-glucans, arable gum modified and resistant starches, polydextrose, D-tagatose, acacia fibers, carob, oats, and citrus fibers. In one aspect, the prebiotics are the short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded.

The compositions of the invention may comprise pharmaceutically acceptable excipients or carriers. Examples of such suitable excipients may be found in the reference [55]. Acceptable carriers or diluents for therapeutic use are well known in the pharmaceutical art and are described, for example, in reference [56]. Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

The compositions of the invention may be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. In certain embodiments, the composition of the invention is formulated as a milk-based product. The term “milk-based product” means any liquid or semi-solid milk- or whey-based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Another important group includes milk beverages, such as whey beverages, fermented milks, condensed milks, infant or baby milks; flavoured milks, ice cream; milk-containing food such as sweets. In certain embodiments, the compositions of the invention contain a single bacterial strain or species and do not contain any other bacterial strains or species. In certain embodiments, the compositions of the invention contain a single bacterial species and do not contain any other bacterial species. In certain embodiments, the compositions of the invention contain a single bacterial strain and do not contain any other bacterial strains. For example, the compositions of the invention may comprise bacteria only of the species Bifidobacterium breve. Such compositions may comprise only de minimis or biologically irrelevant amounts of other bacterial strains or species. Such compositions may be a culture that is substantially free from other species of organism. In some embodiments, such compositions may be a lyophilisate that is substantially free from other species of organism.

In some embodiments, the compositions of the invention comprise more than one bacterial strain or species. For example, in some embodiments, the compositions of the invention comprise more than one strain from within the same species (e.g. more than 1, 2, 3, 4, 5, 6, 7, 8, 9, 10, 15, 20, 25, 30, 35, 40 or 45 strains), and, optionally, do not contain bacteria from any other species. In some embodiments, the compositions of the invention comprise less than 50 strains from within the same species (e.g. less than 45, 40, 35, 30, 25, 20, 15, 12, 10, 9, 8, 7, 6, 5, 4 or 3 strains), and, optionally, do not contain bacteria from any other species. In some embodiments, the compositions of the invention comprise 1-40, 1-30, 1-20, 1-19, 1-18, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same species and, optionally, do not contain bacteria from any other species. In some embodiments, the compositions of the invention comprise more than one species from within the same genus (e.g. more than 1, 2, 3, 4, 5, 6, 7, 8, 9, 10, 12, 15, 17, 20, 23, 25, 30, 35 or 40 species), and, optionally, do not contain bacteria from any other genus. In some embodiments, the compositions of the invention comprise less than 50 species from within the same genus (e.g. less than 50, 45, 40, 35, 30, 25, 20, 15, 12, 10, 8, 7, 6, 5, 4 or 3 species), and, optionally, do not contain bacteria from any other genus. In some embodiments, the compositions of the invention comprise 1-50, 1-40, 1-30, 1-20, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 species from within the same genus and, optionally, do not contain bacteria from any other genus. The invention comprises any combination of the foregoing.

In some embodiments, the composition comprises a microbial consortium. For example, in some embodiments, the composition comprises the Bifidobacterium breve bacterial strain as part of a microbial consortium. For example, in some embodiments, the Bifidobacterium breve bacterial strain is present in combination with one or more (e.g. at least 2, 3, 4, 5, 10, 15 or 20) other bacterial strains from the genus Blautia and/or other genera with which it can live symbiotically in vivo in the intestine. For example, in some embodiments, the composition comprises a bacterial strain of Bifidobacterium breve in combination with a bacterial strain from a different genus. In another example, the composition comprises a bacterial strain of Bifidobacterium breve in combination with a bacterial strain from the genus Bifidobacterium or the composition comprises a bacterial strain of Bifidobacterium breve in combination with a bacterial strain from the genus Bifidobacterium and a bacterial strain from a different genus. In some embodiments, the microbial consortium comprises two or more bacterial strains obtained from a faeces sample of a single organism, e.g. a human. In some embodiments, the microbial consortium is not found together in nature. For example, in some embodiments, the microbial consortium comprises bacterial strains obtained from faeces samples of at least two different organisms. In some embodiments, the two different organisms are from the same species, e.g. two different humans. In some embodiments, the two different organisms are an infant human and an adult human In some embodiments, the two different organisms are a human and a non-human mammal.

In some embodiments, the composition of the invention additionally comprises a bacterial strain that has the same safety and therapeutic efficacy characteristics as the Bifidobacterium breve strain deposited under accession number NCIMB 42380, but which is not the Bifidobacterium breve strain deposited under accession number NCIMB 42380.

In some embodiments in which the composition of the invention comprises more than one bacterial strain, species or genus, the individual bacterial strains, species or genera may be for separate, simultaneous or sequential administration. For example, the composition may comprise all of the more than one bacterial strain, species or genera, or the bacterial strains, species or genera may be stored separately and be administered separately, simultaneously or sequentially. In some embodiments, the more than one bacterial strains, species or genera are stored separately but are mixed together prior to use.

In some embodiments, the bacterial strain for use in the invention is obtained from human adult faeces. In some embodiments in which the composition of the invention comprises more than one bacterial strain, all of the bacterial strains are obtained from human adult faeces or if other bacterial strains are present, they are present only in de minimis amounts. The bacteria may have been cultured subsequent to being obtained from the human adult faeces and being used in a composition of the invention.

In some embodiments, the one or more Bifidobacterium breve bacterial strains is/are the only therapeutically active agent(s) in a composition of the invention. In some embodiments, the bacterial strain(s) in the composition is/are the only therapeutically active agent(s) in a composition of the invention.

The compositions for use in accordance with the invention may or may not require marketing approval.

In certain embodiments, the invention provides the above pharmaceutical composition, wherein said bacterial strain is lyophilised. In certain embodiments, the invention provides the above pharmaceutical composition, wherein said bacterial strain is spray dried. In certain embodiments, the invention provides the above pharmaceutical composition, wherein the bacterial strain is lyophilised or spray dried and wherein it is live. In certain embodiments, the invention provides the above pharmaceutical composition, wherein the bacterial strain is lyophilised or spray dried and wherein it is viable. In certain embodiments, the invention provides the above pharmaceutical composition, wherein the bacterial strain is lyophilised or spray dried and wherein it is capable of partially or totally colonising the intestine. In certain embodiments, the invention provides the above pharmaceutical composition, wherein the bacterial strain is lyophilised or spray dried and wherein it is viable and capable of partially or totally colonising the intestine.

In some cases, the lyophilised or spray dried bacterial strain is reconstituted prior to administration. In some cases, the reconstitution is by use of a diluent described herein.

The compositions of the invention can comprise pharmaceutically acceptable excipients, diluents or carriers.

In certain embodiments, the invention provides a pharmaceutical composition comprising: a bacterial strain of the invention; and a pharmaceutically acceptable excipient, carrier or diluent; wherein the bacterial strain is in an amount sufficient to treat a disorder when administered to a subject in need thereof.

In certain embodiments, the invention provides pharmaceutical composition comprising: a bacterial strain of the invention; and a pharmaceutically acceptable excipient, carrier or diluent; wherein the bacterial strain is in an amount sufficient to treat or prevent a disease or condition.

In certain embodiments, the invention provides pharmaceutical composition comprising: a bacterial strain of the invention; and a pharmaceutically acceptable excipient, carrier or diluent; wherein the bacterial strain is in an amount sufficient to treat or prevent a disease or condition.

In certain embodiments, the invention provides pharmaceutical composition comprising: a bacterial strain of the invention; and a pharmaceutically acceptable excipient, carrier or diluent; wherein the bacterial strain is in an amount sufficient to treat or prevent a disease or condition.

In certain embodiments, the invention provides pharmaceutical composition comprising: a bacterial strain of the invention; and a pharmaceutically acceptable excipient, carrier or diluent; wherein the bacterial strain is in an amount sufficient to treat or prevent a disease or condition mediated a reduced immune response.

In certain embodiments, the invention provides the above pharmaceutical composition, wherein the amount of the bacterial strain is from about 1×10³ to about 1×10¹¹ colony forming units per gram with respect to a weight of the composition.

In certain embodiments, the invention provides the above pharmaceutical composition, wherein the composition is administered at a dose of 1 g, 3 g, 5 g or 10 g.

In certain embodiments, the invention provides the above pharmaceutical composition, wherein the composition is administered by a method selected from the group consisting of oral, rectal, subcutaneous, nasal, buccal, and sublingual.

In certain embodiments, the invention provides the above pharmaceutical composition, comprising a carrier selected from the group consisting of lactose, starch, glucose, methyl cellulose, magnesium stearate, mannitol and sorbitol.

In certain embodiments, the invention provides the above pharmaceutical composition, comprising a diluent selected from the group consisting of ethanol, glycerol and water.

In certain embodiments, the invention provides the above pharmaceutical composition, comprising an excipient selected from the group consisting of starch, gelatin, glucose, anhydrous lactose, free-flow lactose, beta-lactose, corn sweetener, acacia, tragacanth, sodium alginate, carboxymethyl cellulose, polyethylene glycol, sodium oleate, sodium stearate, magnesium stearate, sodium benzoate, sodium acetate and sodium chloride.

In certain embodiments, the invention provides the above pharmaceutical composition, further comprising at least one of a preservative, an antioxidant and a stabilizer.

In certain embodiments, the invention provides the above pharmaceutical composition, comprising a preservative selected from the group consisting of sodium benzoate, sorbic acid and esters of p-hydroxybenzoic acid.

In certain embodiments, the invention provides the above pharmaceutical composition, wherein said bacterial strain is lyophilised.

In certain embodiments, the invention provides the above pharmaceutical composition, wherein when the composition is stored in a sealed container at about 4° C. or about 25° C. and the container is placed in an atmosphere having 50% relative humidity, at least 80% of the bacterial strain as measured in colony forming units, remains after a period of at least about: 1 month, 3 months, 6 months, 1 year, 1.5 years, 2 years, 2.5 years or 3 years.

In some embodiments, the composition of the invention is provided in a sealed container comprising a composition as described herein. In some embodiments, the sealed container is a sachet or bottle. In some embodiments, the composition of the invention is provided in a syringe comprising a composition as described herein.

The composition of the present invention may, in some embodiments, be provided as a pharmaceutical formulation. For example, the composition may be provided as a tablet or capsule. In some embodiments, the capsule is a gelatine capsule (“gel-cap”). The capsule can be a hard or a soft capsule. In some embodiments, the formulation is a soft capsule. Soft capsules are capsules which may, owing to additions of softeners, such as, for example, glycerol, sorbitol, maltitol and polyethylene glycols, present in the capsule shell, have a certain elasticity and softness. Soft capsules can be produced, for example, on the basis of gelatine or starch. Gelatine-based soft capsules are commercially available from various suppliers. Depending on the method of administration, such as, for example, orally or rectally, soft capsules can have various shapes, they can be, for example, round, oval, oblong or torpedo-shaped. Soft capsules can be produced by conventional processes, such as, for example, by the Scherer process, the Accogel process or the droplet or blowing process.

In some embodiments, the compositions of the invention are administered orally. Oral administration may involve swallowing, so that the compound enters the gastrointestinal tract.

Pharmaceutical formulations suitable for oral administration include solid plugs, solid microparticulates, semi-solid and liquid (including multiple phases or dispersed systems) such as tablets; soft or hard capsules containing multi- or nano-particulates, liquids (e.g. aqueous solutions), emulsions or powders; lozenges (including liquid-filled); chews; gels; fast dispersing dosage forms; films; ovules; sprays; and buccal/mucoadhesive patches.

In some embodiments the pharmaceutical formulation is an enteric formulation, i.e. a gastro-resistant formulation (for example, resistant to gastric pH) that is suitable for delivery of the composition of the invention to the intestine by oral administration. Enteric formulations may be particularly useful when the bacteria or another component of the composition is acid-sensitive, e.g. prone to degradation under gastric conditions.

In some embodiments, the enteric formulation comprises an enteric coating. In some embodiments, the formulation is an enteric-coated dosage form. For example, the formulation may be an enteric-coated tablet or an enteric-coated capsule, or the like. The enteric coating may be a conventional enteric coating, for example, a conventional coating for a tablet, capsule, or the like for oral delivery. The formulation may comprise a film coating, for example, a thin film layer of an enteric polymer, e.g. an acid-insoluble polymer.

In some embodiments, the enteric formulation is intrinsically enteric, for example, gastro-resistant without the need for an enteric coating. Thus, in some embodiments, the formulation is an enteric formulation that does not comprise an enteric coating. In some embodiments, the formulation is a capsule made from a thermogelling material. In some embodiments, the thermogelling material is a cellulosic material, such as methylcellulose, hydroxymethylcellulose or hydroxypropylmethylcellulose (HPMC). In some embodiments, the capsule comprises a shell that does not contain any film forming polymer. In some embodiments, the capsule comprises a shell and the shell comprises hydroxypropylmethylcellulose and does not comprise any film forming polymer (e.g. see [57]). In some embodiments, the formulation is an intrinsically enteric capsule (for example, Vcaps® from Capsugel).

Culturing Methods

The bacterial strains for use in the present invention can be cultured using standard microbiology techniques as detailed in, for example, references [58-60].

The solid or liquid medium used for culture may be YCFA agar or YCFA medium. YCFA medium may include (per 100 ml , approximate values): Casitone (1.0 g), yeast extract (0.25 g), NaHCO3 (0.4 g), cysteine (0.1 g), K₂HPO₄ (0.045 g), KH₂PO₄ (0.045 g), NaCl (0.09 g), (NH₄)₂SO₄ (0.09 g), MgSO₄·7H₂O (0.009 g), CaCl₂ (0.009 g), resazurin (0.1 mg), hemin (1 mg), biotin (1 μg), cobalamin (1 μg), p-aminobenzoic acid (3 μg), folic acid (5 μg), and pyridoxamine (15 μg).

Bacterial Strains for use in Vaccine Compositions

The inventors have identified that the bacterial strains of the invention are useful for treating or preventing diseases or conditions associated with reduce immune activity. This is likely to be a result of the effect that the bacterial strains of the invention have on the host immune system. Therefore, the compositions of the invention may also be useful for preventing diseases or conditions, when administered as vaccine compositions. In certain such embodiments, the bacterial strains of the invention may be killed, inactivated or attenuated. In certain such embodiments, the compositions may comprise a vaccine adjuvant. In certain embodiments, the compositions are for administration via injection, such as via subcutaneous injection.

General

The practice of the present invention will employ, unless otherwise indicated, conventional methods of chemistry, biochemistry, molecular biology, immunology and pharmacology, within the skill of the art. Such techniques are explained fully in the literature. See, e.g., references [61] and [62,68], etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

References to a percentage sequence identity between two nucleotide sequences means that, when aligned, that percentage of nucleotides are the same in comparing the two sequences. This alignment and the percent homology or sequence identity can be determined using software programs known in the art, for example those described in section 7.7.18 of ref. [69]. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref [70].

Unless specifically stated, a process or method comprising numerous steps may comprise additional steps at the beginning or end of the method, or may comprise additional intervening steps. Also, steps may be combined, omitted or performed in an alternative order, if appropriate.

Various embodiments of the invention are described herein. It will be appreciated that the features specified in each embodiment may be combined with other specified features, to provide further embodiments. In particular, embodiments highlighted herein as being suitable, typical or preferred may be combined with each other (except when they are mutually exclusive).

All patent and literature references cited in the present specification are hereby incorporated by reference in their entirety.

Any reference to a method for treatment comprising administering an agent to a patient, also covers that agent for use in said method for treatment, as well as the use of the agent in said method for treatment, and the use of the agent in the manufacture of a medicament.

The following examples are offered for illustrative purposes only, and are not intended to limit the scope of the present invention in any way.

### MODES FOR CARRYING OUT THE INVENTION

### Example 1

Summary

The objective of this study was to characterise the in vitro immunomodulatory properties of MRx0004. In addition, a combination of genomics, transcriptomics and proteomics was used to identify potential key effectors which could be responsible for mediating the host response to MRx0004.

Materials and Methods

Bacterial Strains, Plasmids and Primers

All bacterial strains and plasmids and primers used to generate strains in this study are listed in Table 1. B. breve strains were routinely cultured in yeast extract-casein hydrolysate -fatty acids (YCFA) broth (E&O Labs, Bonnybridge, UK) at 37° C. in an anaerobic workstation (Don Whitley Scientific, Shipley, UK) unless otherwise stated. E. coli strains were routinely cultured in Luria Bertani (LB) broth [71] at 37° C. with agitation. Where appropriate, growth media was supplemented with tetracycline (10 μg/ml), chloramphenicol (10 μg/ml for E. coli or 3 μg/ml for B. breve), erythromycin (100 μg/ml for E. coli or 1 μg/ml for B. breve), spectinomycin (100-300 μg/ml), or kanamycin (50 μg/ml) (all antibiotics from Sigma-Aldrich, Gillingham, UK). Recombinant E. coli cells containing pORI19 or pWSK29 were selected on LB agar supplemented with 40 μg/ml X-gal (5-bromo-4-chloro-3-indolyl-β-D-galactopyranoside) and 0.1 M IPTG (isopropyl-β-D-galactopyranoside) (both supplied by Sigma-Aldrich).

Routine Culture of Immortalised Cells

HT29-MTX-E12 cells (Public Health England, Salisbury, UK) were routinely cultured in Dulbecco's Minimal Eagle's Medium (DMEM) with high glucose modification, supplemented with 10% (v/v) foetal bovine serum (FBS), 4 mM L-glutamine, 1× non-essential amino acid solution and 1× antibiotic antimycotic solution. Cells were seeded into assay vessels and cultured for nine days, following which they were washed twice with Hank's Balanced Saline Solution and placed into co-culture medium (DMEM supplemented with 4 mM L-glutamine, 1× non-essential amino acid solution, 5μg/ml apo-transferrin and 200 ng/ml sodium selenite) prior to the beginning of treatments.

Preparation of Bacterial Treatments for Co-Culture Assays

For co-culture experiments, bacteria were cultured until they reached log phase. Live bacterial cells and supernatant were separated by centrifugation, following which live bacteria (designated_{LV}) were washed once with PBS (Sigma-Aldrich) and resuspended in the appropriate cell culture medium for downstream use. Supernatants (designated _{SN}) were passed through a 0.22 μm filter and diluted appropriately in co-culture medium. Heat-inactivated bacteria (designated HO were prepared by incubation at 80° C. for 30 minutes, followed by washing with PBS and resuspension in appropriate cell culture medium. Viable counts were confirmed by plating.

Reporter Assays

HEK-Blue™-hTLR2 and THP1-Blue™ NF-κB cells were grown to 90% density, washed once with PBS and resuspended in culture media without antibiotic at a density of 280,000 and 500,000 cells/ml, respectively. Bacterial treatments (live, heat-killed and supernatants) were added to cells at a multiplicity of infection (MOI) of 100:1. Positive assay controls, PamCS3K4 (Invivogen) and heat-killed L. monocytogenes (HKLM) (Invivogen) were used at 10 ng/ml concentrations and MOI of 200:1, respectively. Negative controls, media and vehicles, were prepared to provide equivalents for each treatment. Cells were then incubated at 37° C. and 5% CO₂ for 22 h. Medium from co-cultures was diluted ten-fold in QUANTI-Blue™ (Invivogen), and incubated for 1 hour (NFκB) or 2 hours (TLR2) and optical density at 655 nm was recorded.

Large Scale Co-Cultures with HT29-MTX Cells

HT29-MTX cells were cultured as described previously, in the upper chamber of 10 cm diameter Transwells® (Thermo Fisher Scientific, Waltham, Mass., USA). Bacteria were cultured to late log phase, washed and resuspended as described previously. Bacteria were added to cells at an MOI of 100:1, and co-cultures were incubated for 3 hours in anaerobic conditions at 37° C. Medium containing bacteria was collected from the upper chamber of the transwell, and centrifuged at 5000×g for 5-10 minutes to collect bacterial cells for downstream applications.

Bacterial qPCR Analysis

Bacteria were collected for RNA isolation from in vitro culture at late log and stationary growth phases, and post-large scale co-culture with HT29-MTX. Bacteria were collected and stored using RNAProtect Bacteria Reagent as per manufacturer's instructions (QIAGEN, Hilden, Germany) Bacterial cells were lysed by incubation with lysozyme (15 mg/ml) (Sigma-Aldrich) and proteinase K (6 mAU) (QIAGEN) at 37° C. for 30 minutes, and subsequently homogenised using a FastPrep 24 instrument (2 x cycles of 6 m/s for 20 s), and Lysing Matrix B (both from MP Biomedicals, Santa Ana, Calif., USA). Total RNA was isolated using an RNeasy Mini Kit (QIAGEN), and genomic DNA was removed using RNase-Free DNase in an on-column digest (QIAGEN), both as per manufacturer's instructions. cDNA was synthesised using a Superscript IV kit (Thermo Fisher Scientific) as per manufacturer's instructions. Primers were designed using Primer3Plus software [72]. qPCR reactions were set up Power SYBRTM Green PCR Master Mix (Thermo Fisher Scientific) as per manufacturer's recommendations, and assays were carried out on a 7500 Fast Real-time PCR System (Thermo Fisher Scientific), using the following cycle: 95° C. for 10 mins, followed by 40 cycles of 95° C. for 15 s, 60° C. for 1 min. Data was analysed was carried out using double delta Ct analysis and expression of test genes was normalised to the housekeeper groEL.

Bacterial Cell Shaving

Bacterial cells were harvested by centrifugation in late log growth phase or following contact with HT29-MTX (see co-culture section above for details), as appropriate. Cells were then washed and resuspended in 50 mM TEAB buffer pH 8.5 (Sigma-Aldrich) at a 1/20 dilution. Shaved protein fractions were generated by incubating cells with sequencing grade modified trypsin (Promega, Madison, Wis., USA) for 30 min at 37° C. in 50 mM TEAB buffer supplemented with 1 mM DTT (Sigma-Aldrich). For each sample, a tube without trypsin was incubated in parallel, as a control for shed proteins (shed protein fraction). Shaved and shed protein fractions were harvested by centrifugation for 15 min at 4000×g at 4° C. and syringe-filtered through a Millex-GV 0.22 μm low protein binding membrane (Millipore). Total protein concentrations were measured using Pierce™ BCA Protein Assay Kit according to the manufacturer's instructions (Thermo Fisher Scientific) and sample quality was assessed by SDS-PAGE (Bio-Rad, Hercules, Calif., USA). Viable cell counts were performed before and after the trypsin treatment by plating on YCFA agar. For each assay, samples from three biological replicates were then analyzed by nano-LC-MS/MS.

Protein Identification by LC-MS/MS

In brief, culture supernatants were concentrated down to 0.5 ml and washed with ultrapure water, proteins were precipitated using a ReadyPrep 2-D Cleanup Kit (Bio-Rad) and resuspended in 100 μl 50 mM ammonium bicarbonate. Samples were then incubated with porcine trypsin (Promega) for 16 h at 37° C. and the resulting supernatants were dried by vacuum centrifugation and dissolved in 0.1% trifluoroacetic acid. Peptides were further desalted using μ-C18 ZipTips (Merck, Keniloworth, N.J., USA) and eluted into a 96-well microtiter plate, dried by vacuum centrifugation and dissolved in 10 μl LC-MS loading solvent (2% acetonitrile, 0.1% formic acid). Peptides were separated and identified by nanoLC-MS/MS (Q Exactive hybrid quadrupole-Orbitrap MS system) (Thermo Fisher Scientific) using a 15-cm PepMap column, 60-minute LC-MS acquisition method and an injection volume of 5 μl. For the shaved and shed protein fractions, 70 μl 50 mM ammonium bicarbonate were directly added to 30 μl of sample. Samples were then incubated with porcine trypsin (Promega) overnight at 37° C. and the resulting supernatants were frozen at −70° C., dried by vacuum centrifugation and dissolved in 20 μL of LC-MS loading solvent. Peptides were separated and identified by nanoLC-MS/MS (Q Exactive hybrid quadrupole-Orbitrap MS system, Thermo Scientific) using a 25-cm PepMap column, 60-minute LC-MS acquisition method, and an injection volume of 2 μL. Data analysis was performed with Proteome Discoverer (Thermo Fisher Scientific). The Mascot Server was used as the search engine with the following parameters: enzyme=trypsin, maximum mixed cleavage sites=2, precursor mass tolerance=10 ppm, dynamic modifications=oxidation (M), static modifications=carbamidomethyl (C). Identified peptides were matched against a strain-specific protein sequence database, which was constructed based on the sequenced genome of B. breve MRx0004 (2,047 sequences). A protein identification was considered valid when at least five peptides were identified in all three biological replicates.

Complementation of B. breve MRx0004-EPS^{neg}

A DNA fragment encompassing the primary glycosyl transferase encoding gene pGTF and its assumed promoter was generated by PCR amplification from B. breve MRx0004 chromosomal DNA using Q5 High-Fidelity Polymerase (New England BioLabs, Herefordshire, United Kingdom) and primer pairs: pGTFcompF and pGTFcompR. The resulting fragment was digested with HinDIII and XbaI (both from New England Biolabs, Ipswich, MA, USA) and ligated to the similarly digested pBC1.2. The ligation mixture was introduced into E. coli EC101 by electrotransformation and transformants were then selected based on Cm resistance. The plasmid content of a number of Cm-resistant transformants was screened by restriction analysis. The integrity of the cloned insert in a number of the recombinant plasmids was confirmed by sequencing prior to their introduction into E. coli EC101 pWSK29-MRX-M+S to facilitate methylation. Methylated pBC1.2 or pBC1.2-pGTF was transformed into MRx0004-EPS^{neg} by electroporation with selection on Reinforced Clostridial Agar (RCA; Thermo Fisher Scientific) supplemented with Tet and Cm. Transformants were checked for plasmid content using colony PCR, restriction analysis of plasmid DNA, and verified by sequencing. The resulting strains were designated B. breve MRx0004-EPS⁻-pBC1.2 and MRx0004- EPS⁻-pBC1.2-pGTF.

Cytokine Analysis from HT29-MTX Cells

Live bacteria (prepared as described previously) were co-incubated with HT29-MTX cells in 24 well plates at an MOI of 100:1 for 3 hours at 37° C. and 5% CO_{2.} Human Recombinant TNFα(PeproTech, Rocky Hill, N.J., USA) was then added to cells at 10 ng/ml, following which co-cultures were incubated for a further 24 hours, and subsequently supernatants were collected and centrifuged at 12000×g for 3 min at 4° C. to remove cell debris. IL-8 levels in supernatants were analysed using a Human IL-8 (CXCL8) Standard ABTS ELISA Development Kit (PeproTech) as per manufacturer's recommendations.

Co-Culture with PBMCs

Healthy frozen human peripheral blood mononuclear cells (PBMCs) were purchased from STEMCELL Technologies (Cambridge, UK). Cells were thawed and left to rest overnight in full growth media; RPMI 1640 with 10% FBS, 2mM L. Glutamine and 100 U/ml penicillin, 100 μg/ml streptomycin at 37° C. and 5% CO₂ (all reagents from Sigma-Aldrich). Bacterial treatments were prepared as previously described. For co-incubations, cells were plated at a density of 750,000 cell/wells in 48 well plates and co-incubated with heat-inactivated bacteria, and bacterial supernatants at an MOI of 10:1, with appropriate vehicles and 5 μg/ml PHA (Sigma-Aldrich) as a control. Co-cultures were incubated for 72 h at 37° C. and 5% CO₂, following which cells were collected and centrifuged at 10000×g for 3 minutes at 4° C. Cell-free supernatants were collected and stored at −80° C. for cytokine analysis. Cell pellets were washed once and then resuspended in PBS on ice.

Flow Cytometry

Treatment wells were pooled to give 1.5×10⁶ PBMCs per group, resuspended in 150 ul PBS and transferred to a 96 V-bottom plate ready for staining. Cells were first stained with the Viobility 405/520 Fixable Dye (Miltenyi Biotec Ltd. Bergisch Gladbach, Germany) to discriminate between live and dead cells for 10 min in the dark at room temperature. They were then stained with a cocktail of antibodies for CD3, CD4, CD8, CD25, CD127 and CD19 to determine cell phenotype (Miltenyi REA antibodies) and incubated for a further 10 min at room temperature. Cells were then washed and resuspended in PBS and immediately analysed via flow cytometric analysis. Isotypes were used for all antibodies during the first experiment to help set gates and FMO controls were included throughout all the experiments. All experiments were performed using a BD FACS Aria II with the stopping gate for acquisition set on 100,000 cells in the “Live” gate using FACSDiva software (BD Biosciences, Reading, UK). The analysis was conducted using Flowjo version 10.4.2 software (FlowJo LLC, Oregon, USA) and was based on live cells identified with the viability dye.

Cytokine Analysis Cytokine quantification in cell-free supernatants from PBMC co-cultures was carried out using custom ProcartaPlex multiplex immunoassays (Thermo Fisher Scientific) following the manufacturer's recommendations. Briefly, 50 μl of supernatants were processed using a MAGPIX® MILLIPLEX® system (Merck) with the xPONENT software (Luminex, Austin, Tex., USA). Data was analysed using the MILLIPLEX® analyst software (Merck) using a 5-parameter logistic curve and background subtraction to convert mean fluorescence intensity (MFI) to pg/ml values.

Data Analysis Statistical analyses were carried out using GraphPad Prism version 7.00 for Windows, GraphPad Software, La Jolla Calif. USA. Data were analysed using one-way ANOVA and Tukey's Multiples Comparisons Test. Venn diagrams were generated using Interactivenn [73].

Results

MRx0004 Stimulates NFκB and TLR2 Reporter Cells

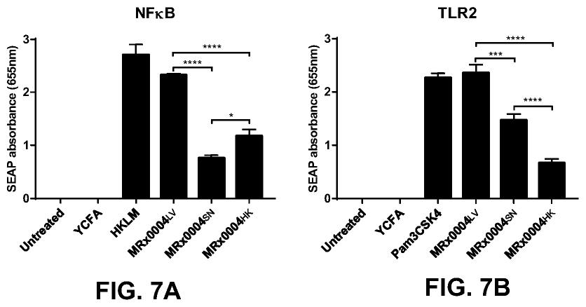

A THP-1-NFκB reporter cell line was employed to examine the impact of MRx0004 on activation of the pro-inflammatory transcription factor NFκB, due to its integral role in the regulation of innate immunity. Heat-killed Listeria monocytogenes (HKLM) (InvivoGen) was used as a positive control for this assay. In order to identify the effective fraction(s) of this strain, THP-1-NFκB cells were co-incubated with treatments of live bacteria (MRx0004_{LV}), bacterial culture supernatant (MRx0004_{SN}) and heat-inactivated bacteria (MRx0004_{HK}). All three bacterial treatments significantly activated NFκB in comparison to negative controls of untreated cells and bacterial growth medium (YCFA) (p<0.0001 for all comparisons) (FIG. 7A). MRx0004_{LV} was the most effective treatment and was significantly more stimulatory than MRx0004_{SN} and MRx0004_{HK} (p<0.0001 for both comparisons). MRx0004_{HK} was in turn significantly more active than MRx0004_{SN} (p=0.006).

As MRx0004 was shown to activate NFκB, the ability of MRx0004 to stimulate the upstream receptors TLR2, TLR4, TLR5 and TLR9 was investigated. Preliminary data suggested that MRx0004 did not activate TLR4, TLR5 and TLR9 (data not shown). A HEK-TLR2 reporter assay was treated with positive control Pam3CSK4 and the same MRx0004 treatments and negative controls as described above (FIG. 7B). All MRx0004 treatments stimulated TLR2 in comparison to negative controls (p<0.0001 for all comparisons). MRx0004Lv was the most stimulatory treatment compared to MRx0004_{SN} (p<0.0001) and MRx0004_{HK}(p<0.0001), the latter of which was the least effective treatment. Based on these data, MRx0004 is capable of potently stimulating the innate immune response in a TLR2-associated manner.

Transcriptional and Proteomic Profiling of MRx0004 Reveals Potential Host Response Effectors

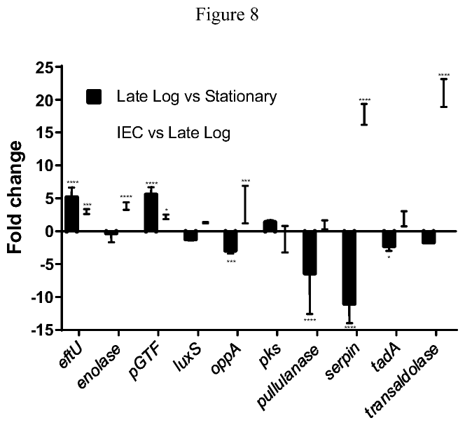

In order to identify potential effectors of the host response in MRx0004, three approaches were employed. A targeted transcriptional assay was employed to analyse ten MRx0004 genes with predicted identities as known effectors of bifidobacterial-host interaction (data not shown). These included genes coding for predicted adhesins and moonlighting proteins (oppA, enolase, transaldolase, tadA, eftU, pullulanase (reviewed extensively in [74]), and genes with putative roles in colonisation and immune modulation (the primary glycosyltransferase (pGTF) of the MRx0004 EPS locus [75], luxS [76] and serpin [77]) and putative therapeutic effects (pks [78]). Expression of these genes was analysed in RNA isolated from MRx0004 grown to late log phase and stationary phase in liquid culture, and after 3 h contact with HT29-MTX cells cultured in a large-scale transwell. qPCR analysis (FIG. 8) demonstrated that eftU, enolase and pGTF were significantly upregulated in late log phase compared to stationary phase, whereas the expression of oppA, pullulanase, serpin and tadA was significantly higher in stationary phase than in late log phase. Six of the genes (eftU, enolase, pGTF, oppA, serpin and transaldolase) were significantly upregulated in response to intestinal epithelial cells (IECs) relative to in late log phase. It was evident from this analysis that gene expression of MRx0004 was altered by contact with IECs, inferring a potential role for the upregulated genes in MRx0004-host interactions. Most of the significantly upregulated genes in this qPCR analysis have predicted roles in adhesion to IECs, which suggests that this might be an important functional property of MRx0004.

The expression of additional host-response effectors by MRx0004 was further characterised by identifying proteins present in culture supernatant and on the cell surface. Nano-LC-MS/MS analysis identified 64 proteins in MRx0004_{SN} (Table 2). The proteins identified with the highest number of peptides matched (PSM) were pullulanase ((351.33±33.62), two NlpC/P60 family proteins (82±15.62 and 71.67±13.61), a solute-binding protein of ABC transporter system (56±7) and the cell division protein Ftsl (56±4.36). Several moonlighting proteins involved in host-interaction in B. breve and other bacterial species were identified, which included transaldolase (32.67±3.79), GAPDH (30 ±3.61), DnaK (17.67±3.21), GroEL (12.67±0.58) and enolase (5.67±0.58) [74, 79-84].

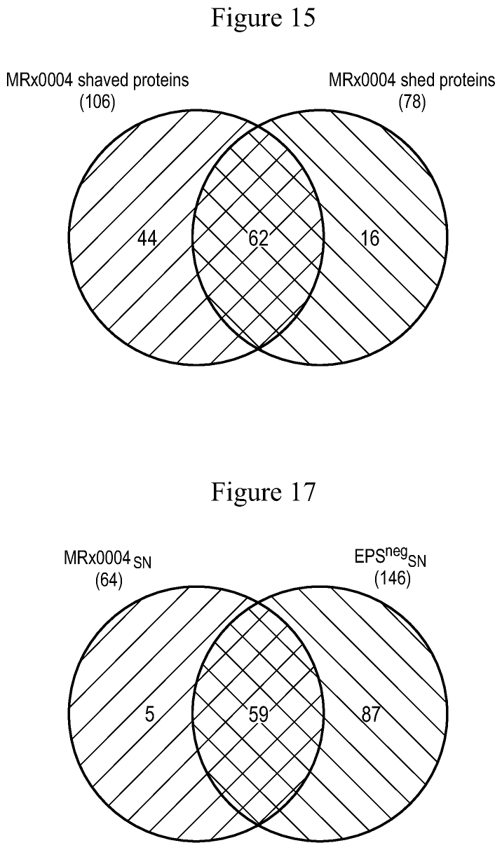

Identification of proteins present on the surface of MRx0004 cells was performed using an enzymatic cell-shaving approach. Bacterial cells were shaved using trypsin and cleaved surface-associated proteins were identified using LC-MS/MS (shaved protein fraction). Proteins from no-trypsin controls were also harvested and analysed by LC-MS/MS, allowing identification of proteins loosely bound to the surface (shed protein fraction). A total of 106 shaved proteins were identified, 44 of which were predicted to be anchored in the cell wall (i.e. present in the shaved protein fraction and absent from the shed protein fraction), (Table 3, FIG. 15). As observed for MRx0004s_{N}, the most abundant shaved protein identified was pullulanase (136.67±17.47), which was also detected in the shed proteins fractions (79±10.54). Interestingly, a type I polyketide synthase was also identified in both cell shaving fractions, although more matching peptides were identified in the shaved protein fraction than in the shed protein fraction (54.67±10.69 and 11.67±6.43 respectively). All moonlighting proteins present in MRx0004_{SN} and listed above were also detected in MRx0004 cell shaving fractions (Table 3). In addition, EfTu was identified in both the shaved and shed protein fractions (PSM=32±5 and 24.33±8.39 respectively). Five of the genes analysed by qPCR (eftU, enolase, luxS, pullulanase and transaldolase) were also detected in one or both of the cell shaving and supernatant datasets, thus confirming the translation of these genes. The data from transcriptional and proteomic datasets collectively allowed us to identify potential novel effectors of interest in MRx0004.

Interestingly, the amylolytic enzyme pullulanase, the most abundant protein in the cell shaving dataset, is a moonlighting protein which has been reported to be involved in the adhesion of Streptococcus pyogenes to glycoproteins and host cells in vitro [85,86]. In addition, DnaK and enolase from B. animalis subsp. lactis [82,83] and EfTu from B. longum [87] have been reported to adhere to plasminogen in vitro. Furthermore, recombinant expression of the glycolytic enzyme transaldolase of B. bifidum A8 in L. lactis increased the adherence of this strain to mucin [84]. The production of these moonlighting proteins by MRx0004 suggests that they may play a role in the adhesive capacity of MRx0004, and thus facilitate interaction with the host cell surface. The effects of bifidobacterial moonlighting proteins on specific host cell receptors and signalling pathways have not yet been described, and these may represent novel regulatory pathways for MRx0004 through which MRx0004 interacts with the host.

Investigating the Characteristics of Modulation of the Innate Immune Response by MRx0004

Further experiments were performed to characterise the host response towards MRx0004.

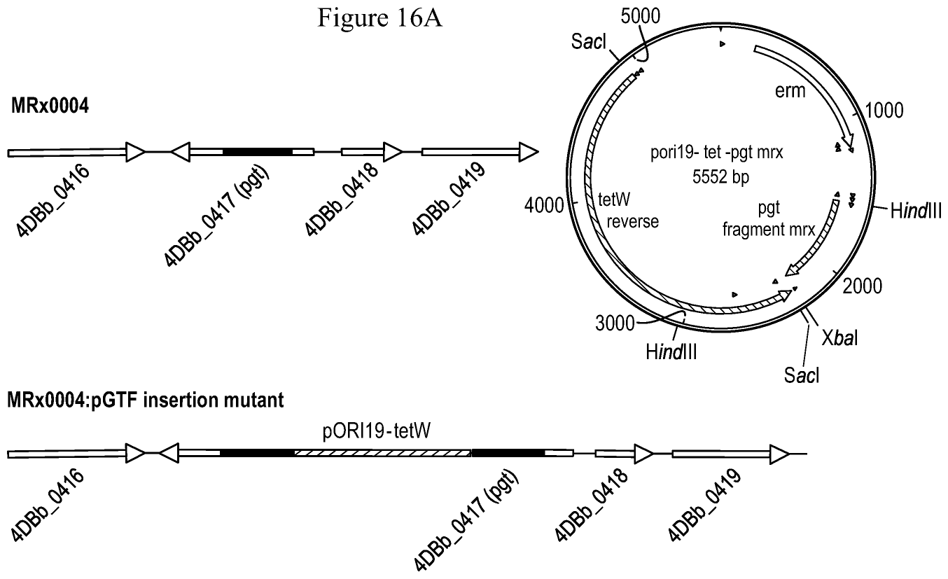

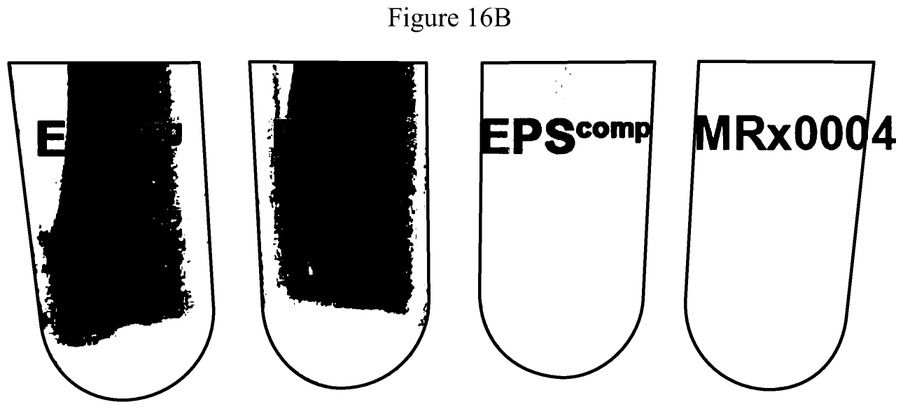

To assist this study, a strain (EPS^{neg}) was constructed whereby the pGTF gene of the EPS locus was inactivated through insertional mutagenesis (FIG. 16A). This strain was constructed by utilising the methodology described in [88], but rather than manipulating a Type II restriction-modification (RM) system, the methylase and specificity subunits from the MRx0004 Type I RM system were expressed and used to methylate plasmid DNA prior to transformation. A complemented EPS^{neg} strain (EPS^{comp}) and an EPS^{neg} empty vector strain (EPS^{vec}) were also generated as controls.

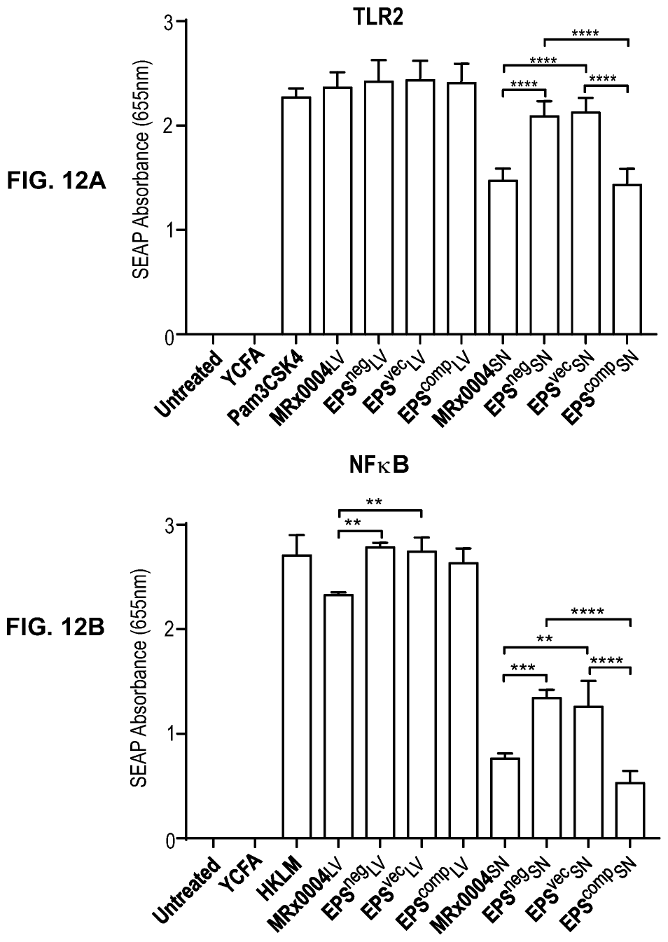

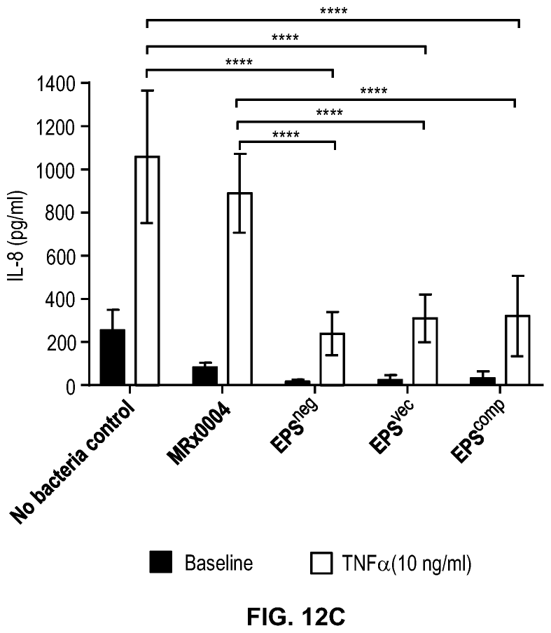

The impact of EPS on TLR2 and NFκB activation was assessed by co-incubating HEK-TLR2 and THP-1-NFκB reporter cells with live and supernatant treatments of MRx0004, EPS^{neg}, EPS^{vec} and EPS^{comp}. All live bacterial treatments activated TLR2 to a comparable extent (FIG. 12A). In contrast, the activation of NFκB by MRx0004Lv was significantly lower than that of EPS^{neg}_{LV} (p=0.003) and EPS^{vec} (p=0.009), but not EPS^{comp}_{LV} (FIG. 12B). There was no difference in TLR2 and NFκB activation between MRx0004_{SN} and EPS^{comp}_{SN}, demonstrating that in this instance the wild type and complement displayed a similar phenotype. EPS^{comp}_{SN} and EPS^{vec}_{SN} were significantly more stimulatory towards TLR2 (p<0.0001 for both) and NFκB (p=0.002 and 0.0013 respectively) than MRx0004_{SN} These data suggest that TLR2 activation by MRx0004 is not mediated directly through its EPS. In contrast, the activation of NFκB is increased in response to the exposure of the MRx0004 cell surface in the absence of EPS. These data suggest that effective immunomodulation by MRx0004 is best achieved using the whole, intact cell, and that the MRx0004 ligand for TLR2 is primarily cell surface-associated, but may also be shed or secreted.

The impact of MRx0004 and its derivative strains on an in vitro model of IEC inflammation was also investigated. HT29-MTX cells were primed with MRx0004 and its derivatives for 3 hours, following which TNFα was added to wells as an inflammatory stimulant for a further 24 hours. Using this model, MRx0004 did not reduce TNFα-mediated IL-8 secretion in comparison with a no bacteria control (FIG. 12C). However, IL-8 secretion in non-TNFa-stimulated cells was decreased by MRx0004 treatment compared to a baseline control. EPS^{neg} treatment resulted in a significant reduction of TNFα-induced IL-8 secretion compared to that of MRx0004 (p<0.0001). IL-8 secretion in EPS^{vec}- and EPS^{comp}-treated cells was also significantly lower than in response to MRx0004 (p<0.0001 for both comparisons). Interestingly, it appeared that the unshielding of surface-associated antigens had an anti-inflammatory effect on IECs, in contrast to the effects demonstrated by EPS^{neg} in reporter assays.

Impact of MRx0004 on the Adaptive Immune Response

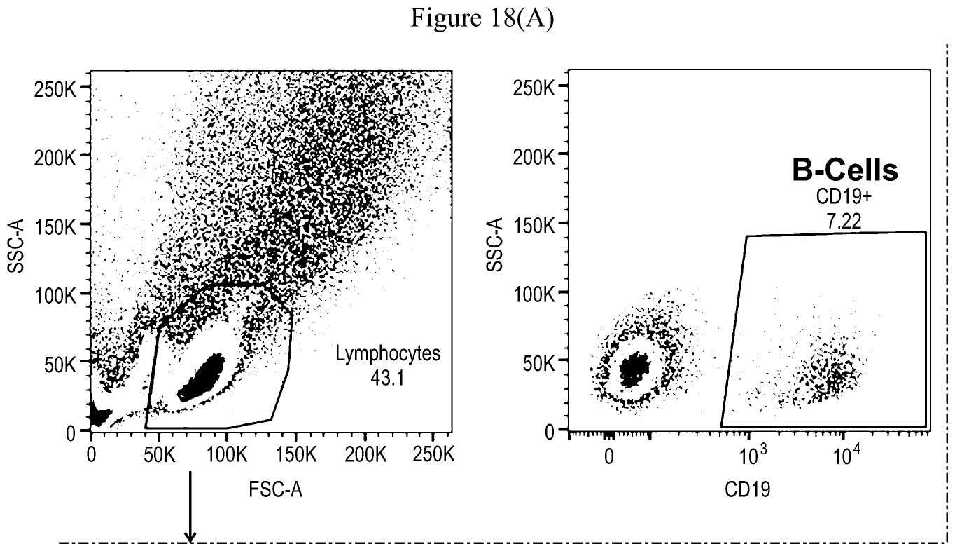

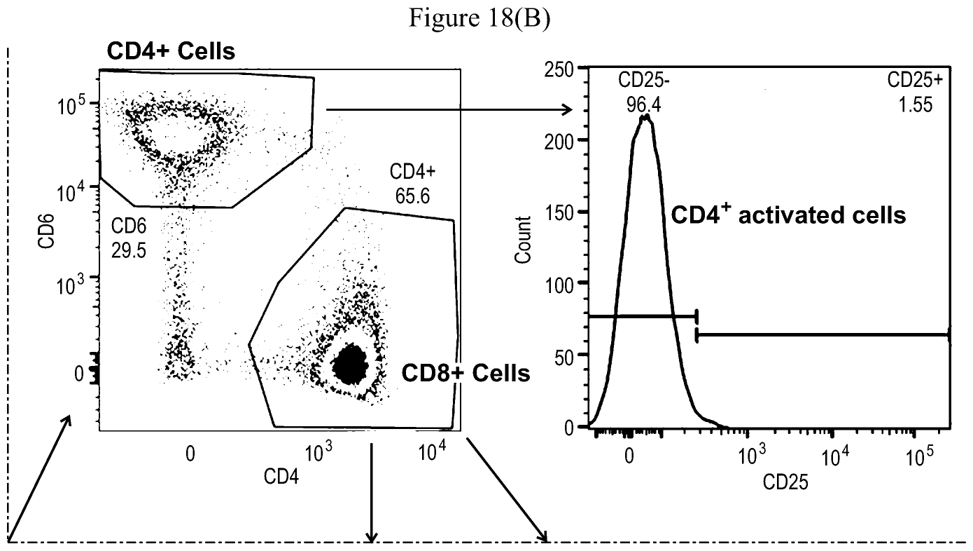

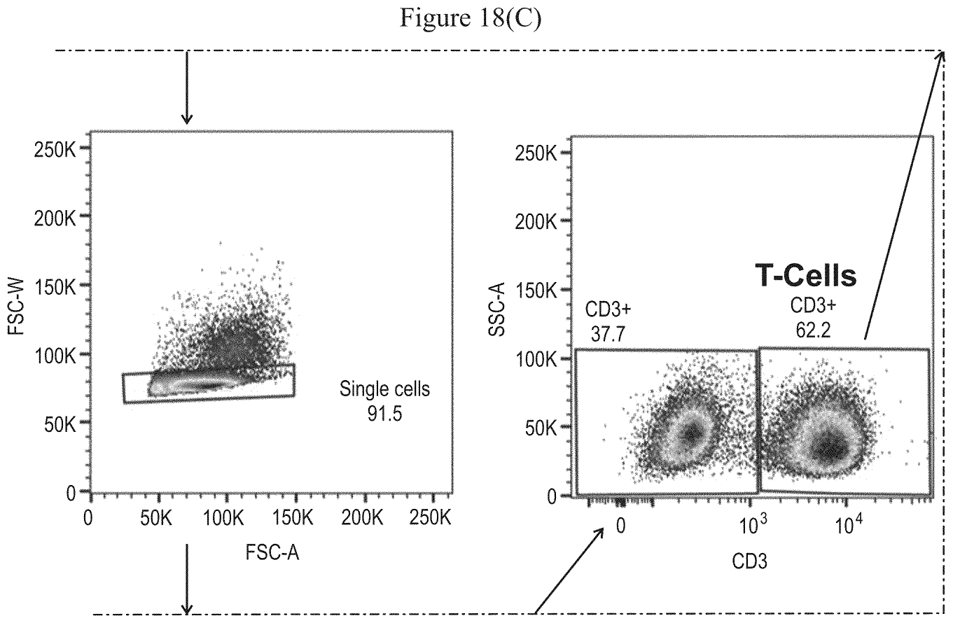

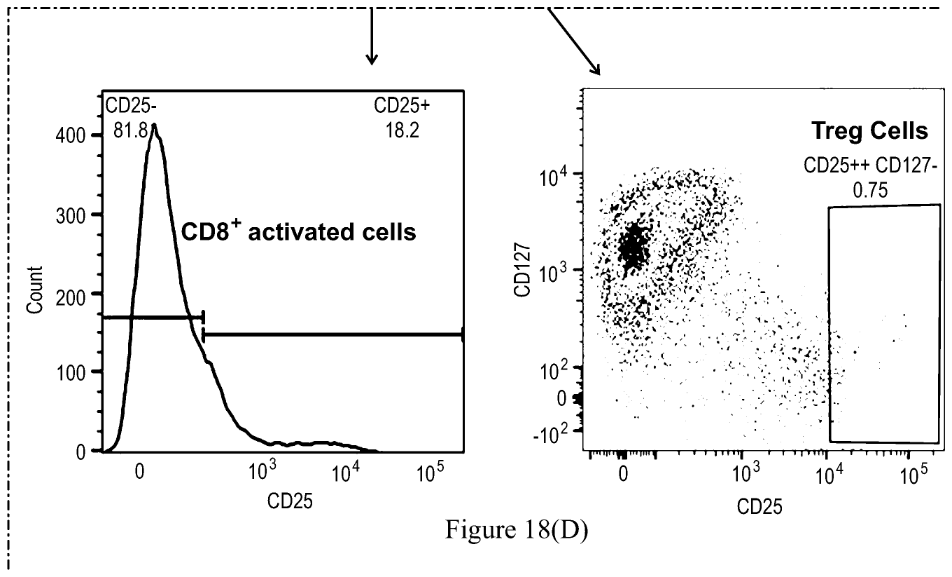

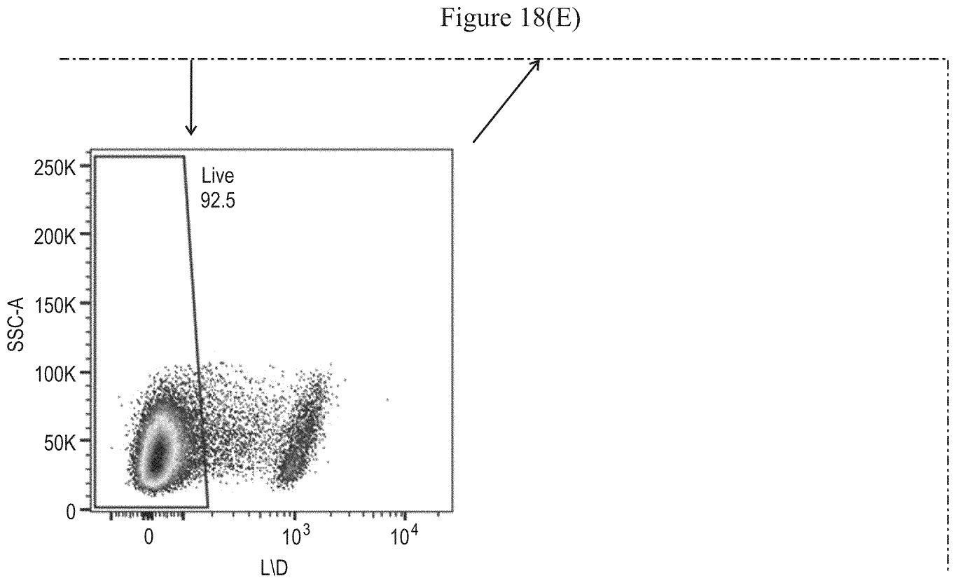

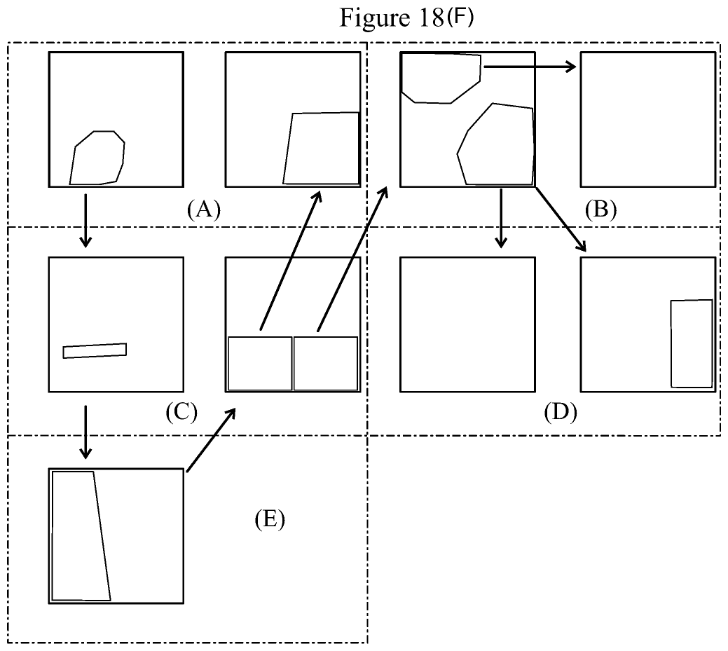

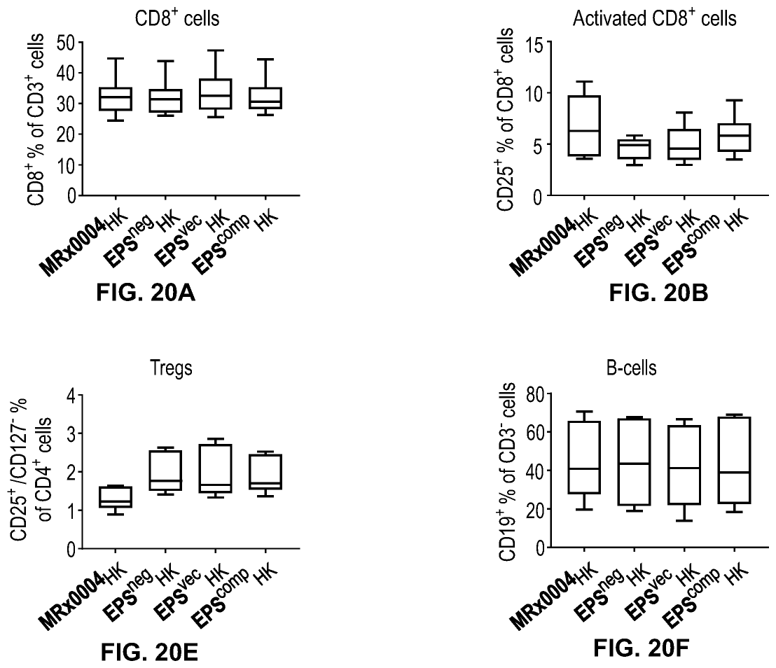

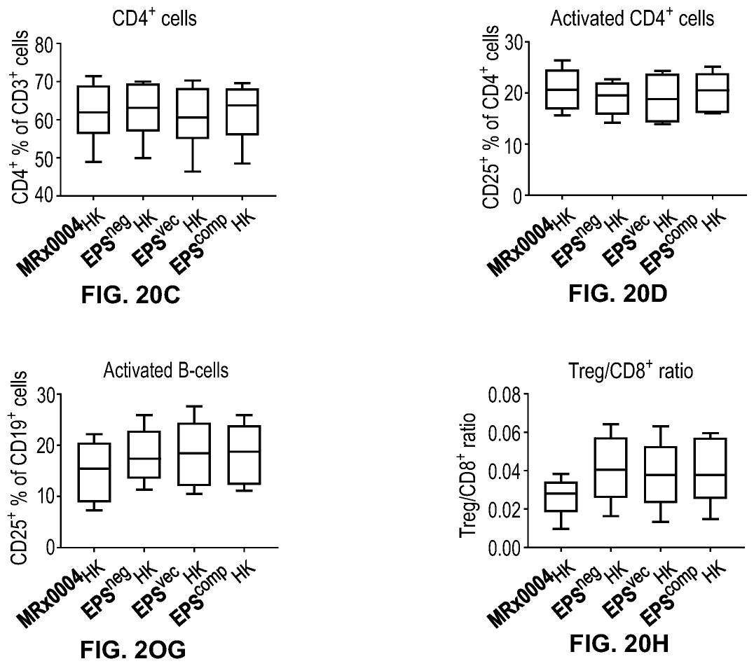

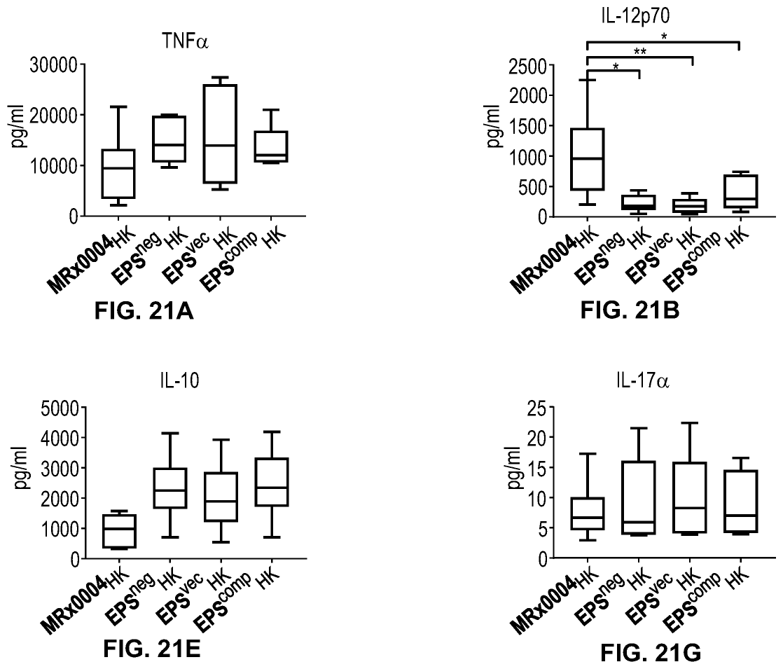

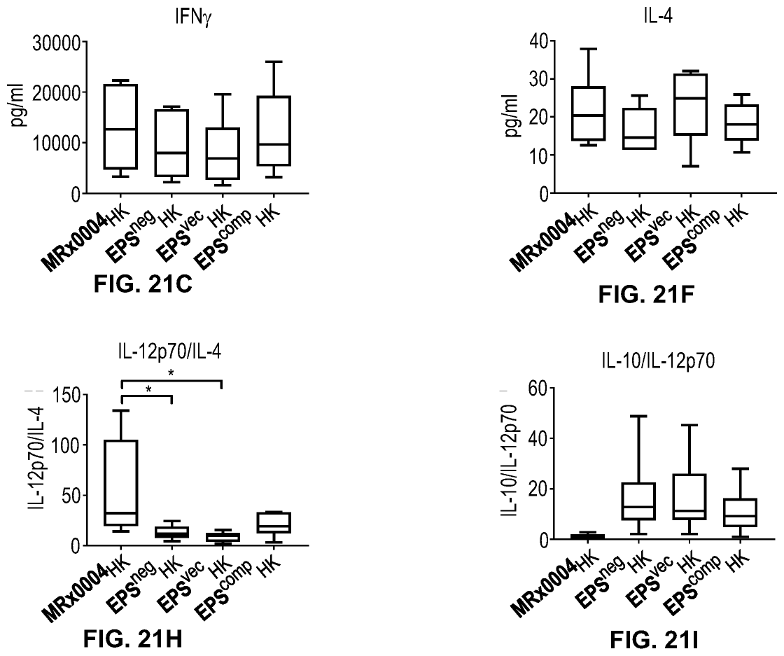

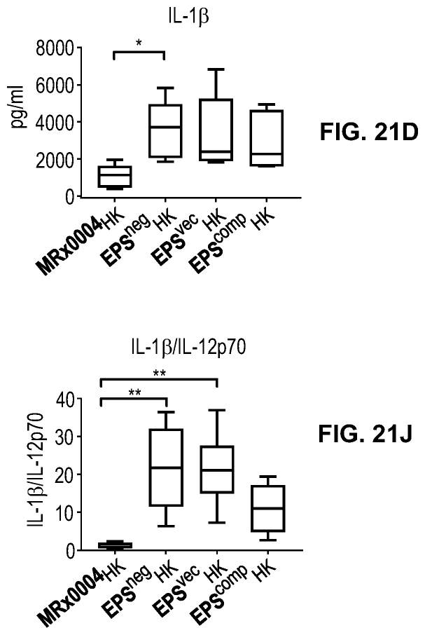

To examine the effect of MRx0004 on the adaptive immune system, peripheral blood mononuclear cells (PBMCs) from healthy human donors were used to characterise cell populations and cytokine secretion profiles. PHA was used as a positive control in this assay (data not shown). PBMCs were co-incubated with heat-inactivated bacterial cells and cell-free culture supernatants from MRx0004 and its derivative strains for 72 hours. The expression of T-cell (CD3⁺CD4⁺ and CD3⁺CD8⁺), Treg (CD3⁺CD4⁺CD25⁺CD127⁻) and B-cell (CD3⁻CD19⁺) surface markers were analysed (along with activation marker CD25) by flow cytometry (refer to FIG. 18 for gating strategy). Heat-inactivated rather than live bacteria were used as treatments in this model due to the probability that live bacteria would grow and outcompete human cells for nutrients during the 72 h incubation period. The expression of both the cell surface markers and cytokines in response to bacterial supernatants from all strains tested was not significantly different compared to that observed in response to the vehicle (YCFA) (data not shown). Data for EPS^{vec} and EPS^{comp} are illustrated in FIGS. 20 and 21.

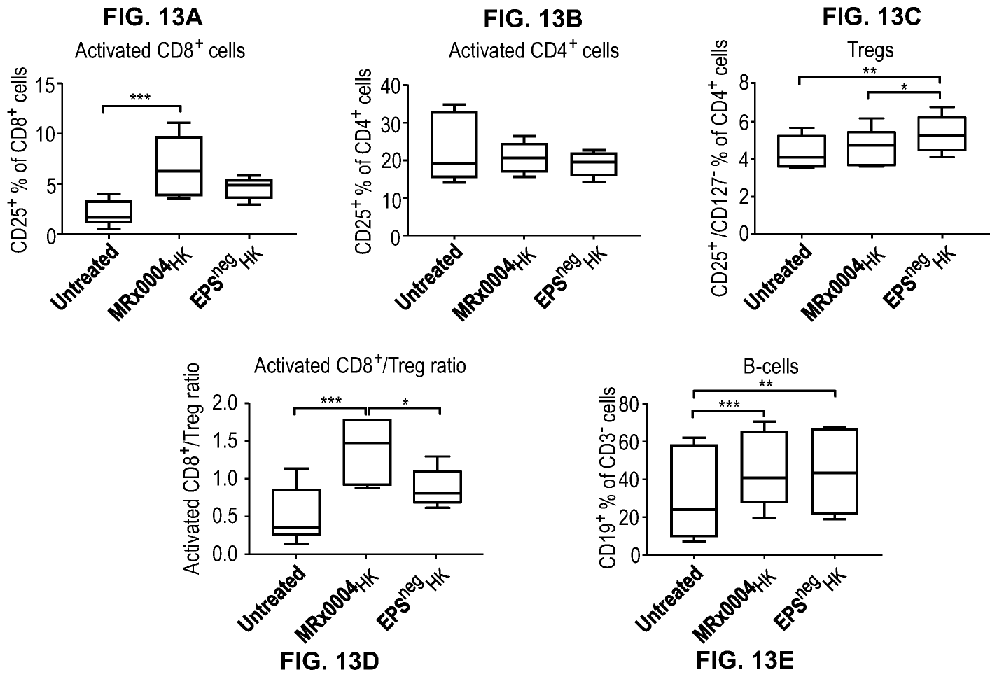

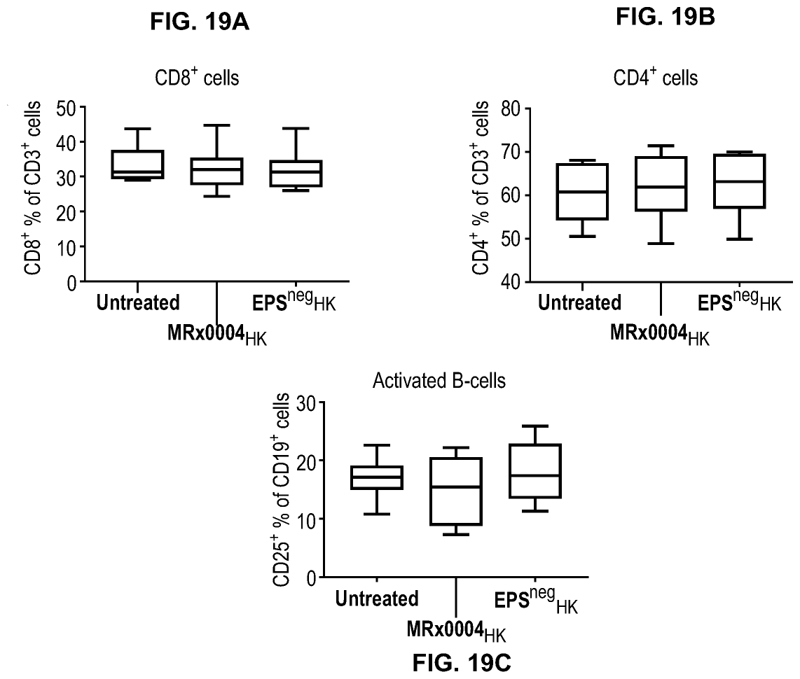

MRx0004_{HK} treatment resulted in a significant increase in activated CD8⁺CD25⁺ subsets compared to the untreated control (p=0.0038, FIG. 13A), whilst EPS^{neg}_{HK} did not significantly increase the percentage of activated CD8⁺CD25⁺ cells compared to controls. Neither MRx0004_{HK} nor EPS^{neg}_{HK} significantly increased the percentage of CD8⁺, CD4⁺ or CD4⁺ CD25⁺ T-cell populations compared to controls (FIG. 19A and B, FIG. 13B). The B cell population was increased by a similar, statistically significant extent by both MRx0004_{HK} and EPS^{neg}_{HK} in comparison to untreated cells (p=0.001 and 0.0013 respectively, FIG. 13E). B-cell activation (CD19⁺CD25⁺) was not significantly affected by any of the applied treatments (FIG. 19-C). Within the CD4⁺ cell population, the proportion of Treg cells was analysed using the CD25⁺CD127⁻surface markers. An increase in the relative percentage of Tregs was observed in the EPS^{neg}_{HK} treated PBMCs but not the MRx0004_{HK} PBMCs (p=0.0014, FIG. 13C) when compared to untreated cells. EPS^{neg}_{HK} increased Tregs relative to MRx0004_{HK} (p=0.0196). A skew in the Treg/CD8⁺ ratio towards a regulatory T cell response was observed in for MRx0004_{HK} treatment in comparison to untreated cells (p=0.0008, FIG. 13D. Furthermore, the increased CD8⁺ positive skew in response to MRx0004_{HK} was significantly higher than that towards EPS^{neg}_{HK} (p=0.0272, FIG. 13D). Taken together, these data confirm the immunostimulatory effect of MRx0004 and suggest that the loss of EPS may result in an increased stimulation of Tregs and the Treg/CD8⁺ ratio and a reduced immunostimulatory effect. Whilst EPS may play a role in the activation of CD8⁺ cells it does not seem to be involved in significantly modulating the B-cell population.

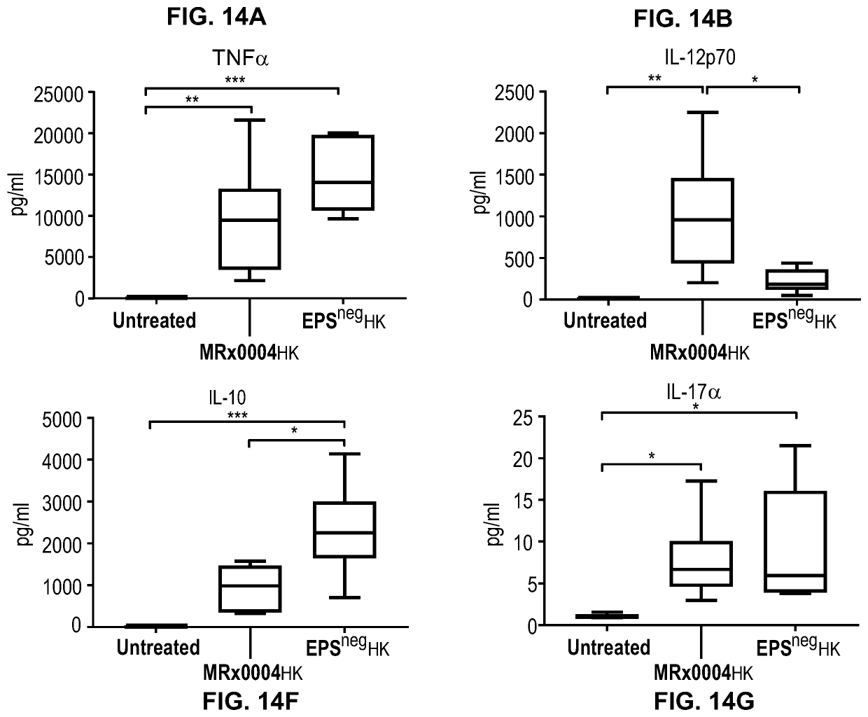

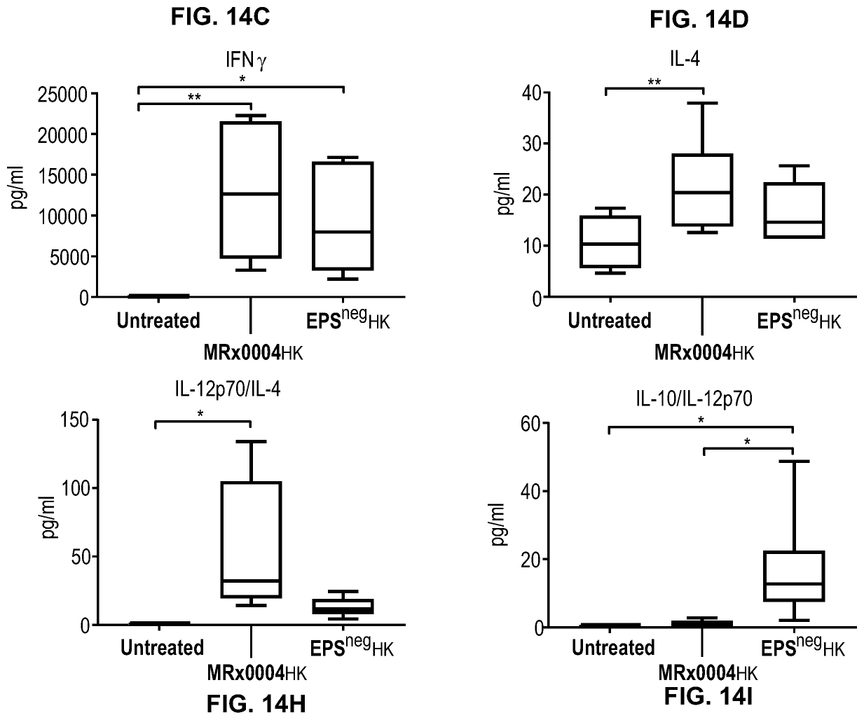

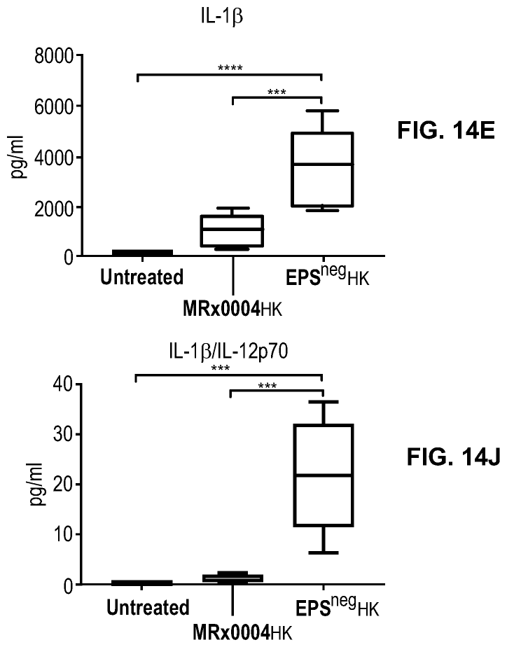

The secreted cytokine signature of PBMCs treated with MRx0004_{HK} and EPS^{neg}_{HK} was also determined, by quantifying cytokines mostly associated with Th1 (IL-12p70, IFNγ, TNFα), Th2 (IL-4), Th17 (IL-17α, IL-1β), and Treg (IL-10) populations. TNFα, IL-12p70, IFNγ, IL-4 and IL-17α were significantly increased by MRx0004_{HK} treatment compared to untreated cells (p=0.0038, 0.0025, 0.0036, 0.027 and 0.0316 respectively, FIG. 14A-D). Treatment with EPS^{neg}_{HK} induced a significant response in TNFα, IFNγ, IL-1β, IL-10 and IL-17α (p=0.0001, 0.0267, <0.0001, 0.0004, and 0.0103 respectively, FIG. 14A, C, E-G). In contrast to MRx0004_{HK}, EPS^{neg}_{HK} did not significantly increase IL-12p70 or IL-4 secretion in comparison to untreated cells. MRx0004_{HK} also significantly increased IL-12p70 in comparison to EPS^{neg}_{HK} (p=0.0118, FIG. 14B), whilst conversely EPS^{neg}_{HK} treatment was found to produce a higher concentration of IL-1β and IL-10 than MRx0004_{HK} (p=0.0008 and 0.014 respectively, FIG. 14E-F).

Cytokine ratios were analysed in order to infer whether bacterial treatments skewed the T-helper cell response towards a particular subtype using cytokines produced by each individual T-helper cell subtype as indicators (Th1 or Th2; IL-12p70/IL-4, Treg; IL-10/IL1p70, Th17; IL-1β/IL12p70, FIG. 14H-J). MRx0004_{HK} treatment appeared to significantly skew the immune response towards a Th1 phenotype compared to untreated cells (p=0.0172, FIG. 14H), whilst EPS^{neg}_{HK} treatment appeared to induce a skew towards both a Treg and Th17 response in comparison to untreated cells (p=0.0312 and 0.0005, FIG. 14I-J). The EPS^{neg}_{HK} Treg and Th17 skew was also significantly increased in comparison to MRx0004_{HK} (p=0.0423 and 0.0008, FIG. 14J). A clear distinction was observed between MRx0004_{HK} and EPS^{neg}_{HK} treatments in their ability to drive different subsets of the T cell response.

Taken together, the observations in this study demonstrate that MRx0004 regulates the pro-inflammatory arms of the innate and adaptive immune response. Therefore, MRx0004 and other B. breve strains may be useful for stimulating the immune system and treating diseases associated with decreased immune activity.

### Example 2—characterising the effect of the MRx0004 eps locus on potency

Summary

The objective of this study was to characterise the role of MRx0004 exopolysaccharide (EPS) in the immunostimulatory and therapeutic properties of MRx0004.

Materials and Methods

Experiments were performed as described in Example 1, with the additional procedures described below.

Routine Culture of Immortalised Cells

HEK-Blue™-hTLR2 cells (InvivoGen, San Diego, Calif, USA) were grown in DMEM supplemented with 10% (v/v) FBS, 4 mM L-glutamine, 4.5 mg/ml glucose, 100 U/ml penicillin, 100 μg/ml streptomycin, 100 μg/ml Normocin™ (InvivoGen), 30 μg/ml blastocydin and 100 μg/ml zeocin to 90% density. THP1-Blue^{tm} NF-kB cells (InvivoGen) were grown in RPMI 1640 supplemented with 10% (v/v) heat-inactivated FBS, 2 mM L-glutamine, 100 U/ml penicillin, 100 μg/ml streptomycin, 25 mM HEPES, 100 μg/ml Normocin™, 10 μg/ml blastocydin (cRPMI). Cell lines were cultured at 37° C. and 5% CO₂. All reagents were supplied by Sigma-Aldrich unless otherwise stated.

Comparative Analysis of MRx0004 EPS Locus and Related Strains of B. breve

Genomes of Bifidobacterium strains available in the GenBank database used for the in silico analysis of EPS clusters and physical maps of the putative exopolysaccharide gene clusters from Bifidobacterium strains.

Differential Expression Analysis of the MRx0004 EPS Locus

Total RNA was extracted late log phase cultures of strain MRx0004 with RNAprotect (Qiagen) and the RNeasy Mini kit (Qiagen), according to the manufacturer's protocol with minor modifications. Mechanical cell lysis was performed using Lysing Matrix B and a MP Fast-Prep-24 tissue and cell homogenizer (MP Biomedicals, Santa Ana, Calif., USA) with oscillations set at 6 m/s. Cells were disrupted for two 20 s cycles with a 1 min rest on ice between cycles. RNA quality was checked on a Tapestation (Agilent Technologies, Santa Clara, Calif., USA) with the Agilent RNA Screentape (Agilent Technologies). The absence of RNA degradation was checked, and all samples had a minimum RNA Integrity Numbers ≥9. MICROBExpress kit (Thermo Fisher Scientific) was used to deplete rRNA species. The absence of 16S and 23S rRNA species was assessed on were checked on an Agilent Bioanalyzer with the Agilent RNA RNA Screentape (Agilent Technologies). RNA samples depleted in rRNA were sent to GATC Biotech for strand-specific library preparation and sequenced on an Illumina sequencing to produce 150 bp single-end reads. An average of 22,3878,08 (late log samples) and 18627178.6 (stationary phase samples) raw reads per RNA-Seq library were obtained totaling over 10.07 Gbp and 8.38 Gbp respectively. Raw reads were trimmed using Trimmomatic (1) and quality filtered (98.36% late log samples and 98.26% (stationary phase samples) reads passed QC and were aligned 99.11% (LL) and 98.72% (SP) of clean reads mapped) to the MRx0004 genome using Bowtie (2). The expression levels of the replicate samples of each growth phase were calculated for each gene in the MRx0004 EPS locus using XX and DeSeq2 v X (Love et al, 2014) and subsequently visualized using Geneious R11 (Biomatters, Auckland, New Zealand). Differential expression between the two growth phases are represented. The base 2 logarithm of the ratio of the normalized values between the two samples and when one sample has no or very low expression, the log2 ratio is capped at +/− 1,000,000.

Transmission Electron Microscopy (TEM)

Bacteria were diluted 1:5 in fixative solution (0.5 M sucrose in 0.1 M Na-phosphate buffer, 2% paraformaldehyde and 0.16% glutaraldehyde) and fixed for 2 hours at room temperature. Thereafter, Formvar-carbon-coated copper grids were floated on 100 μdroplets of the B. breve suspensions for 1 hour, washed three times with 0.02 M glycine in PBS. The cells were negatively stained with 1.0% ammonium molybdate. The grids were examined, and micrographs visualized, using a JEM-1400 transmission electron microscope (JEOL Ltd., Tokyo, Japan).

Bacterial Adhesion Assays

Live bacteria (prepared and resuspended in co-culture medium as described previously) were applied to HT29-MTX cells in 24 well plates at an MOI of 100:1, and co-incubated for 3 hours at 37° C. in anaerobic conditions. Cells were washed twice with PBS to remove unbound bacteria, and lysed with 0.1% (v/v) Triton X-100 (Sigma-Aldrich). Lysate was plated, and the number of colony forming units (CFU) recovered was used to determine the percentage of adhesion.

Results

The eps locus of MRx0004 is genetically distinct from other strains of B. breve and is highly expressed during late log growth.

Genome sequencing of strain MRx0004 indicated that it harboured a 28 Kb EPS locus which was found to encode 27 genes, representing the full complement of functions predicted to be required for EPS biosynthesis in B. breve. This region includes: a priming glycosyltransferase, four additional glycosyltransferases, a thiamine pyrophosphate binding protein which is encoded downstream of a membrane spanning protein, a flippase and a chain-length determinant (FIG. 9). The majority of B. breve EPS loci, including that of strain MRx0004 are flanked by hypothetical proteins (extending the MRx0004 region to 31.5 Kb), which have been excluded from the representation of the B. breve EPS loci represented in FIG. 9. The majority (16/19) of strains illustrated in FIG. 9 are infant isolates, the genomes of which are over-represented in public databases. B. breve EPS regions which exceed 50 Kb are thought to represent complete loci which encode all the functions required to produce EPS-positive phenotypes [89]. In contrast, when these regions are <30 Kb, they are thought to represent incomplete or remnant loci [89].

Comparative analyses identified the EPS locus as a major region of genetic divergence between the genome of strain MRx0004 (data not shown) and other strains of B. breve. The MRx0004 EPS locus was compared to those of the publicly available B. breve genomes. Strains of B. breve whose EPS loci displayed high levels of sequence identity (ID) and gene synteny with that of strain MRx0004 are illustrated in FIG. 9, and are ordered according to their similarity (mean % nt ID over the length of the operon) to strain MRx0004. B. breve NRBB51, an infant (breast fed) isolate shared the highest level of nucleotide identity (ID), 91.5%, with strain MRx0004 over the complete length of the two loci. A central 1.3 Kb region, encoding predicted transposases in both strains, represents the primary region of diversity between strains MRx0004 and NRBB51. The genes encoded at the start and end of the MRx0004 locus displayed the highest level of sequence conservation with other B. breve isolates (FIG. 9). The pGTF of MRx0004, which is essential for the primary step of EPS biosynthesis, shared 92.2% pairwise ID (aa) with homologs in the comparator strains. A 4 Kb region encompassing a chain length regulator, and genes encoding a hypothetical and a membrane spanning protein share 98.3% nt ID among the strains examined. Genes encoding hypothetical proteins and transposases accounted for the major regions of sequence diversity between strain MRx0004 and the ten most closely related EPS loci (FIG. 9). In contrast, the B. breve EPS loci that were found to be more dissimilar to that of strain MRx0004 (the bottom nine strains in FIG. 9) display variance in the number and order of genes that are of central importance to the production of EPS, including gtf, polymerase and acetyltransferase genes (FIG. 9).

In order to determine whether the EPS locus of MRx0004 was more highly transcribed during late log or stationary phase growth, differential expression analysis was carried out on mono-cultures of strain MRx0004 grown in YCFA. (FIG. 9B). The majority of genes which are predicted to be primarily responsible for MRx0004 EPS synthesis (described above), were upregulated during late log phase growth. The MRx0004 EPS locus encodes ten hypothetical proteins and five transposases, which represented the only categories of genes which were upregulated during stationary phase growth of MRx0004 (FIG. 9). The role of the hypothetical proteins in the synthesis of MRx0004 EPS is as yet unknown.

Generation of an EPS Negative Strain ofMRx0004

The role of MRx004 EPS in host-microbe interactions and immunomodulation was investigated and a strain (EPS^{neg}) was constructed, as discussed above, whereby the pGTF gene of the EPS locus was inactivated through insertional mutagenesis (FIG. 16A). This strain was constructed by utilising the methodology described in [88], but rather than manipulating a Type II restriction-modification (RM) system, the methylase and specificity subunits from the MRx0004 Type I RM system were expressed and used to methylate plasmid DNA prior to transformation. A complemented EPS^{neg} strain (EPS^{comp}) and an EPS^{neg} empty vector strain (EPS^{vec}) were also generated as controls. EPS^{neg} and EPS^{vec} displayed an increased autoagreggative phenotype compared with MRx0004 (FIG. 16B). EPS^{comp} was less aggregative than EPS^{neg} and EPS^{vec}, but its autoagreggation appeared to be increased compared to MRx0004, suggesting that this strain may not be fully reverting to a wild type EPS phenotype.

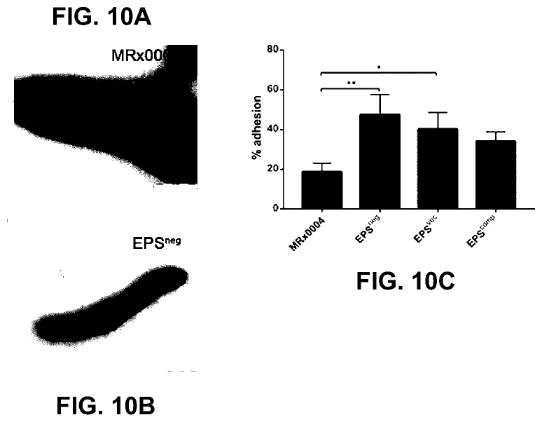

The EPS phenotype of MRx0004 and EPS^{neg} was investigated using transmission electron microscopy (TEM). The absence of EPS in the EPS^{neg} strain compared with MRx0004 is illustrated in FIG. 10A and 10B. The adhesive capabilities of wild type MRx0004 and its derivative strains were analysed using an in vitro IEC model. EPS^{neg} was over twice as adherent to IECs as MRx0004 (47.5% adherence vs 18.7% respectively, p=0.006) (FIG. 10C), suggesting that the absence of EPS increased the adhesive capacity of EPS^{neg}. The increased adhesion phenotype seen in EPS^{neg} was maintained with EPS^{vec} (40.1% adhesion, p=0.03 vs MRx0004), but the adhesion of EPS^{comp} (34.1%) was not significantly different when compared to any other strain, further implying that the reversion of EPS^{comp} to a wild type phenotype was incomplete.

EPS Depletion in MRx0004 Exposes Surface Proteins Involved in Host Stimulation

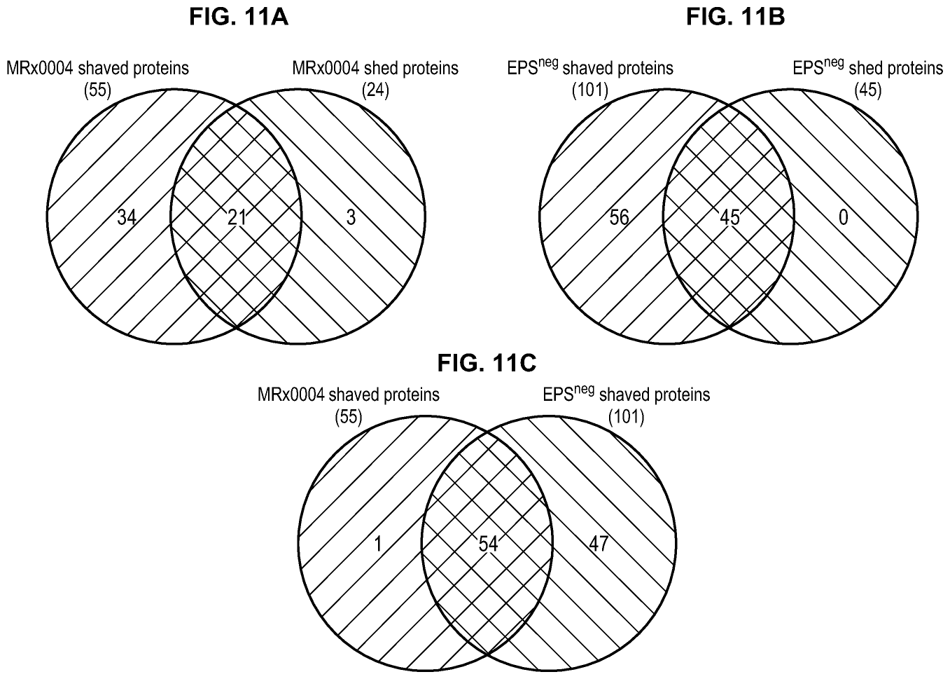

The increased adhesion of EPS^{neg} to IECs suggested that the depletion of EPS may have resulted in the increased exposure of surface-associated proteins, or “unshielding”. The composition of MRx0004 and EPS^{neg} surface-associated proteins was analysed after contact with IECs. Following 3 h contact with HT29-MTX cells, bacterial cells were shaved with trypsin, as described previously, and the resulting shaved and shed protein fractions were analysed by LC-MS/MS. MRx0004 cell shaving after contact with IECs yielded 55 shaved proteins (34 of which were predicted to be surface-anchored) and 24 shed proteins (FIG. 11A). The shaving of EPS^{neg} cells after contact with IECs contained considerably more proteins than that of MRx0004, with 101 proteins in the shaved protein fraction and 45 in the shed protein fraction (FIG. 11B). With the exception of three proteins identified in the MRx0004 shed protein fraction (enzymes involved in carbohydrates, lipid and protein metabolism), all proteins identified were present in the shaved protein fraction for both strains.

Comparison of MRx0004 and EPS^{neg} shaved protein fractions identified 54 proteins that were present in both samples and 47 proteins that were specific to the EPS^{neg} strain (FIG. 11C). The only protein that was uniquely identified in the MRx0004 shaved protein dataset was an N1pC/P60 family protein. The number of proteins harvested by cell shaving was higher for the EPS^{neg} strain than for MRx0004, inferring that EPS depletion facilitated better access to surface proteins for trypsin cleavage. Additionally, proteins known to be involved in host-interaction in bifidobacteria and other genera were more abundant (as assessed by the number of peptides identified/μg total protein) in the EPS^{neg} shaved protein fraction than in that of MRx0004 (Table 4). These results add further credence to the hypothesis that EPS depletion resulted in an unshielding effect, exposing surface proteins and potential MRx0004 immunogens which would be otherwise masked by MRx0004 EPS.

The protein content of EPS^{neg} culture supernatant was also analysed by LC-MS/MS and confirmed that the lack of EPS resulted in an increase in the numbers of proteins potentially shed and secreted in the extracellular milieu by EPS^{neg} strain (FIG. 17). A total of 146 proteins were identified in EPS^{neg}_{SN} in contrast to only 64 in MRx0004_{SN}, of which 87 were detected only in EPS^{neg}_{SN} and 59 in both samples. Moonlighting proteins identified in MRx0004_{SN} and discussed above were all detected in higher abundance in EPS^{neg}_{SN}, with the exception of GAPDH which was comparable between both samples (30±3.61 and 28.33 ±0.58 in MRx0004_{SN} and EPS^{neg}_{SN}). Interestingly, choloylglycine hydrolase (bile salt hydrolase) and EfTu, which have been shown to play a role in human plasminogen binding in B. lactis and B. longum, were detected exclusively in EPS^{neg}_{SN} [80,87]. Bile salt hydrolase may also protect commensal species from environmental stresses in the gut [90]. The increased detection of proteins in EPS^{neg}_{SN} compared to MRx0004_{SN} suggests that unshielding in the absence of EPS may result in the increased shedding or secretion of MRx0004 surface-associated proteins.

Conclusions

Comparative genomic analyses demonstrated that the locus responsible for EPS synthesis in MRx0004 is genetically distinct from other B. breve strains. The genetic variance observed in this region may contribute to the enhanced potency and therapeutic utility of MRx0004 and related strains.

The potential importance of EPS as a mediator of MRx0004:host interactions and as an effector of immune responses was supported by the relative increase in expression of its pGTF upon contact with IECs. In addition to pGTF, most of the other significantly upregulated genes in qPCR analysis had predicted roles in adhesion to IECs, implying that this might be an important functional property of MRx0004. The Type IV pilus-associated gene tadA of MRx0004 was expressed in in vitro culture, which was interesting due to the previous observation that the Tad pilus of B. breve UCC2003 was not produced under in vitro conditions [91]. The expression of serpin was also significantly increased in response to IECs. A serpin from B. longum NCC2705 has recently been reported to reduce the infiltration of intra-epithelial lymphocytes in the small intestine of an in vivo coeliac disease model [92], thus inducing an immunostimulatory and protective effect, which suggests that the serpin of B. breve, and MRx0004 in particular, might have similar immunomodulatory effects.

Proteomics analysis of supernatants and cell shavings detected a large number of proteins with predicted roles in carbohydrate metabolism. Interestingly, the amylolytic enzyme pullulanase, the most abundant protein in the cell shaving dataset, is a moonlighting protein which has been reported to be involved in the adhesion of Streptococcus pyogenes to glycoproteins and host cells in vitro [85,86]. In addition, DnaK and enolase from B. animalis subsp. lactis [82,83] and EfTu from B. longum [87] have been reported to adhere to plasminogen in vitro. Furthermore, recombinant expression of the glycolytic enzyme transaldolase of B. bifidum A8 in L. lactis increased the adherence of this strain to mucin [84]. The production of these moonlighting proteins by MRx0004 suggests that they may play a role in the adhesive capacity of MRx0004, and thus facilitate interaction with the host cell surface. These bifidobacterial moonlighting proteins may have particular effects on specific host cell receptors and signalling pathways that mediate the enhanced effects of MRx0004 on the immune system.

MRx0004 induced a significant increase in activated CD8⁺ subsets, which appeared to be partially associated with the presence of EPS.

EPS^{neg} treatment significantly increased Tregs in comparison to MRx0004. This suggests that the removal of EPS exposes another bacterial surface component capable of interacting with host cells and promoting a Treg response. Fluctuations were evident in both the CD8+and Treg populations. EPS^{neg} induced a significant skew towards a Treg response as illustrated by the significantly increased Treg/CD8 ratio in comparison to baseline. This implies that unshielding of the cell surface in EPS^{neg} results in an anti-inflammatory effect, and the presence of EPS in MRx0004 supports its immunostimulatory potency.

The EPS of MRx0004 was found to be directly involved in secretion of IL-12p70. In addition, secretion of all three of the Th1 cytokines tested (IL-12p70, IFNγ, TNFα) were significantly upregulated by MRx0004_{HK}, suggesting that this strain induces a skew towards a Th1 response which is partially mediated through its EPS. MRx0004i also significantly induced the Th2 cytokine IL-4, and upregulated IL-10, IL-1β and IL-17α, though not significantly, thus inferring that this strain may induce shifts in the T-helper cell microenvironment. The induction of a Th1 may improve intestinal barrier stability in vivo and be beneficial for maintenance of immune homeostasis.

The EPS of MRx0004 may have specific immunoregulatory effects, namely, regulation of the CD8+, Treg and Th1 responses, and these effects may provide enhanced potency and therapeutic efficacy.

### Example 3 —Efficacy of Bacterial Inocula in Mouse Models of Cancer

### Summary

As set out in the preceding examples, the inventors have identified a new immunostimulatory effect of B. breve, and in particular strain MRX004. In light of the new data presented above, compositions comprising B. breve, and in particular strain MRX004, are expected to be effective for stimulating the immune system and treating diseases that are associated with decreased immune system activity or that benefit from increased immune system activity.

Cancer is a disease that may benefit from increased immune system activity attacking tumours. Consistent with the new data presented above and the new immunostimulatory effect of MRX004, MRX004 is shown in the study below to potently reduce tumour volume in mouse tumour models, which demonstrates that administration of MRX004 is effective to treat disease.

This study tested the efficacy of compositions comprising bacterial strains according to the invention in four tumor models, and compared the efficacy to an anti-CTLA antibody.

Materials

Test substance—Bacterial strain #MRX004, Bifidobacterium breve.

Reference substance—Anti-CTLA-4 antibody (clone: 9H10, catalog: BE0131, isotype: Syrian Hamster IgGl, Bioxcell).

Test and reference substances vehicles—Bacterial culture medium (Yeast extract, Casitone, Fatty Acid medium (YCFA)). Each day of injection to mice, antibody was diluted with PBS (ref: BE14-516F, Lonza, France).

Treatment doses—Bacteria: 2×10⁸ in 200 μL. The a-CTLA-4 was injected at 10 mg/kg/inj. Anti-CTLA-4 was administered at a dose volume of 10 mL/kg/adm (i.e. for one mouse weighing 20 g, 200 μL of test substance will be administered) according to the most recent body weight of mice.

Routes of administration—Bacterial inoculum was administered by oral gavage (per os, PO) via a cannula. Cannulas were decontaminated every day. Anti-CTLA-4 was injected into the peritoneal cavity of mice (Intraperitoneally, IP).

Culture conditions of bacterial strain—The culture conditions for the bacterial strain were as follows:
Pipette 10 mL of YCFA (from the prepared 10 mL E&O lab bottles) into Hungate tubes
Seal the tubes and flush with CO₂ using a syringe input and exhaust system
Autoclave the Hungate tubes
When cooled, inoculate the Hungate tubes with 1 mL of the glycerol stocks
Place the tubes in a static 37° C. incubator for about 16 hours.
The following day, take 1 mL of this subculture and inoculate 10 mL of YCFA (pre-warmed flushed Hungate tubes again, all in duplicate)
Place them in a static 37° C. incubator for 5 to 6h

Cancer Cell Line and Culture Conditions

The cell lines that were used are detailed in the table below:

| Cell line | Type | Mouse strain | Origin |
|---|---|---|---|
| EMT-6 | Breast carcinoma | BALB/c | ATCC |
| LL/2 (LLC1) | Lung carcinoma | C57BL/6 | ATCC CRL1642 |
| Hepa1-6 | Hepatocellular | C57BL/6 | IPSEN INNOVATION |
| | carcinoma | | |

The EMT-6 cell line was established from a transplantable murine mammary carcinoma that arose in a BALB/cCRGL mouse after implantation of a hyperplastic mammary alveolar nodule [93].

The LL/2 (LLC1) cell line was established from the lung of a C57BL mouse bearing a tumor resulting from an implantation of primary Lewis lung carcinoma [94].

The Hepa 1-6 cell line is a derivative of the BW7756 mouse hepatoma that arose in a C57/L mouse [95].

Cell culture conditions—All cell lines were grown as monolayer at 37° C. in a humidified atmosphere (5% CO₂, 95% air). The culture medium and supplement are indicated in the table below:

| Cell line | Culture medium | Supplement |
|---|---|---|
| EMT6 | RPMI 1640 containing 2 mM | 10% fetal bovine serum |
| | L-glutamine (ref: BE12-702F, | (ref: #3302, Lonza) |
| | Lonza) | |
| LL/2 (LLC1) | RPMI 1640 containing 2 mM | 10% fetal bovine serum |
| | L-glutamine (ref: BE12-702F, | (ref: #3302, Lonza) |
| | Lonza) | |
| Hepa1-6 | DMEM (ref: 11960-044, Gibco) | 10% fetal bovine serum |
| | | (ref: #3302, Lonza) |
| | | 2 mM L-Glutamine |
| | | penicillin-streptomycin |
| | | (Sigma G- 6784) |

For experimental use, adherent tumor cells were detached from the culture flask by a 5 minute treatment with trypsin-versene (ref: BE17-161E, Lonza), in Hanks' medium without calcium or magnesium (ref: BE10-543F, Lonza) and neutralized by addition of complete culture medium. The cells were counted in a hemocytometer and their viability will be assessed by 0.25% trypan blue exclusion assay.

Use of Animals

Healthy female Balb/C (BALB/cByJ) mice, of matching weight and age, were obtained from CHARLES RIVER (L'Arbresles) for the EMT6 model experiments.

Healthy female C57BL/6 (C57BL16J) mice, of matching weight and age, were obtained from CHARLES RIVER (L'Arbresles) for the LL/2(LLC1) and the Hepa1-6 model experiments.

Animals were maintained in SPF health status according to the FELASA guidelines, and animal housing and experimental procedures according to the French and European Regulations and NRC Guide for the Care and Use of Laboratory Animals were followed [96,97]. Animals were maintained in housing rooms under controlled environmental conditions: Temperature: 22±2° C., Humidity 55±10%, Photoperiod (12h light/12h dark), HEPA filtered air, 15 air exchanges per hour with no recirculation. Animal enclosures were provided with sterile and adequate space with bedding material, food and water, environmental and social enrichment (group housing) as described: 900 cm² cages (ref: green, Tecniplast) in ventilated racks, Epicea bedding (SAFE),10 kGy Irradiated diet (A04-10, SAFE), Complete food for immuno-competent rodents-R/M-H Extrudate, water from water bottles.

Experimental Design and Treatments

Antitumor Activity, EMT6 Model

Treatment schedule—The start of first dosing was considered as D0. On D0, non-engrafted mice were randomized according to their individual body weight into groups of 9/8 using Vivo manager® software (Biosystemes, Couternon, France). On D0, the mice received vehicle (culture medium) or bacterial strain. On D14, all mice were engrafted with EMT-6 tumor cells as described below. On D24, mice from the positive control group received anti-CTLA-4 antibody treatments.

The treatment schedule is summarized in the table below:

| | No. | | | | Treatment |
|---|---|---|---|---|---|
| Group | Animals | Treatment | Dose | Route | Schedule |
| 1 | 8 | Untreated | — | — | — |
| 2 | 8 | Vehicle (media) | — | PO | Q1D × 42 |
| 3 | 9 | Bacterial strain #1 | 2 × 108 | PO | Q1D × 42 |
| | | (MRX004) | bacteria | | |
| 4 | 8 | Anti-CTLA4 | 10 mg/kg | IP | TW × 2 |

The monitoring of animals was performed as described below.

Induction of EMT6 tumors in animals—On D14, tumors were induced by subcutaneous injection of 1×10⁶ EMT-6 cells in 200 μL RPMI 1640 into the right flank of mice.

Euthanasia—Each mouse was euthanized when it reached a humane endpoint as described below, or after a maximum of 6 weeks post start of dosing.

Antitumor Activity, LL/2 (LLC1) Model

Treatment schedule—The start of first dosing was considered as D0. On D0, non-engrafted mice were randomized according to their individual body weight into 7 groups of 9/8 using Vivo manager® software (Biosystemes, Couternon, France). On D0, the mice will received vehicle (culture medium) or bacterial strain. On D14, all mice were engrafted with LL/2 tumor cells as described below. On D27, mice from the positive control group received anti-CTLA-4 antibody treatments.

The treatment schedule is summarized in the table below:

| | No. | | | | Treatment |
|---|---|---|---|---|---|
| Group | Animals | Treatment | Dose | Route | Schedule |
| 1 | 8 | Untreated | — | — | — |
| 2 | 9 | Vehicle (media) | — | PO | Q1D × 42 |
| 3 | 9 | Bacterial strain #1 | 2 × 10⁸ | PO | Q1D × 42 |
| | | (MRX004) | bacteria | | |
| 4 | 8 | Anti-CTLA4 | 10 mg/kg | IP | TW × 2 |

The monitoring of animals was performed as described below.

Induction of LL/2 (LLC1) tumors in animals—On D14, tumors were induced by subcutaneous injection of 1×10⁶ LL/2 (LLC1) cells in 200 μL RPMI 1640 into the right flank of mice.

Euthanasia—Each mouse was euthanized when it reached a humane endpoint as described below, or after a maximum of 6 weeks post start of dosing.

Antitumor Activity, Hepal-6 Model

Treatment schedule—The start of first dosing was considered as DO. On DO, non-engrafted mice were randomized according to their individual body weight into 7 groups of 9 using Vivo manager® software (Biosystemes, Couternon, France). On D0, the mice received vehicle (culture medium) or bacterial strain. On D14, all mice were engrafted with Hepa 1-6 tumor cells as described below. On D16, mice from the positive control group received anti-CTLA-4 antibody treatments.

The treatment schedule is summarized in the table below:

| | No. | | | | Treatment |
|---|---|---|---|---|---|
| Group | Animals | Treatment | Dose | Route | Schedule |
| 1 | 9 | Untreated | — | — | — |
| 2 | 9 | Vehicle (media) | — | PO | Q1D × 42 |
| 4 | 9 | Bacterial strain #2 | 2 × 10⁸ | PO | Q1D × 42 |
| | | (MRX004) | bacteria | | |
| 7 | 9 | Anti-CTLA4 | 10 mg/kg | IP | TW × 2 |

The monitoring of animals was performed as described below.

Orthotopic induction of Hepa 1-6 tumor cells in animals by intrasplenic injection—On D14, one million (1×10⁶) Hepa 1-6 tumor cells in 50 μL RPMI 1640 medium were transplanted via intra-splenic injection into mice. Briefly, a small left subcostal flank incision was made and the spleen was exteriorized. The spleen was exposed on a sterile gauze pad, and injected under visual control with the cell suspension with a 27-gauge needle. After the cell inoculation, the spleen was excised.

Euthanasia—Each mouse was euthanized when it reached a humane endpoint as described in section below, or after a maximum of 6 weeks post start of dosing.

Evaluation of tumor burden at euthanasia—At the time of termination, livers were collected and weighed.

Animal Monitoring

Clinical monitoring—The length and width of the tumor was measured twice a week with callipers and the volume of the tumor was estimated by this formula [98]:

$$Tumor volume = \frac{{width}^{2} \times length}{2}$$

Humane endpoints [99]: Signs of pain, suffering or distress: pain posture, pain face mask, behaviour; Tumor exceeding 10% of normal body weight, but non-exceeding 2000 mm³; Tumors interfering with ambulation or nutrition; Ulcerated tumor or tissue erosion; 20% body weight loss remaining for 3 consecutive days; Poor body condition, emaciation, cachexia, dehydration; Prolonged absence of voluntary responses to external stimuli; Rapid laboured breathing, anaemia, significant bleeding; Neurologic signs: circling, convulsion, paralysis; Sustained decrease in body temperature; Abdominal distension.

Anaesthesia—Isoflurane gas anesthesia were used for all procedures: surgery or tumor inoculation, i.v. injections, blood collection. Ketamine and Xylazine anesthesia were used for stereotaxia surgical procedure.

Analgesia—Carprofen or multimodal carprofenlbuprenorphine analgesia protocol were adapted to the severity of surgical procedure. Non-pharmacological care was provided for all painful procedures. Additionally, pharmacological care not interfering with studies (topic treatment) were provided at the recommendation of the attending veterinarian.

Euthanasia—Euthanasia of animals was performed by gas anesthesia over-dosage (Isoflurane) followed by cervical dislocation or exsanguination.

Results

Antitumor Activity, EMT6 Model

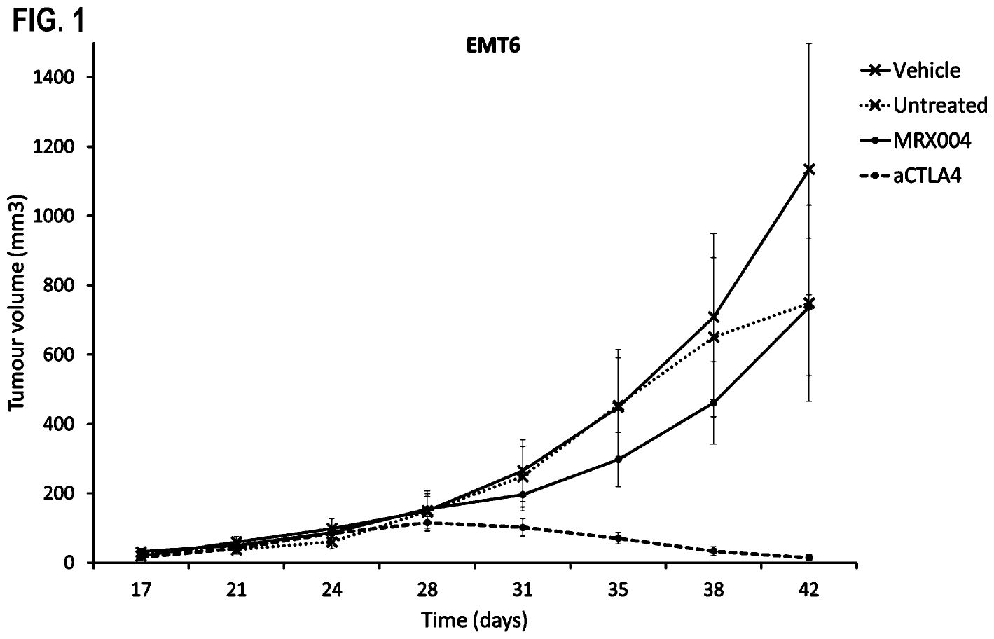

The results are shown in FIG. 1. Treatment with the bacterial strain of the invention led to a clear reduction in tumour volume relative to both the negative controls. The positive control, which is known to activate the immune system, also led to a reduction in tumour volume, as would be expected.

Antitumor Activity, LL/2 (LLC1) Model

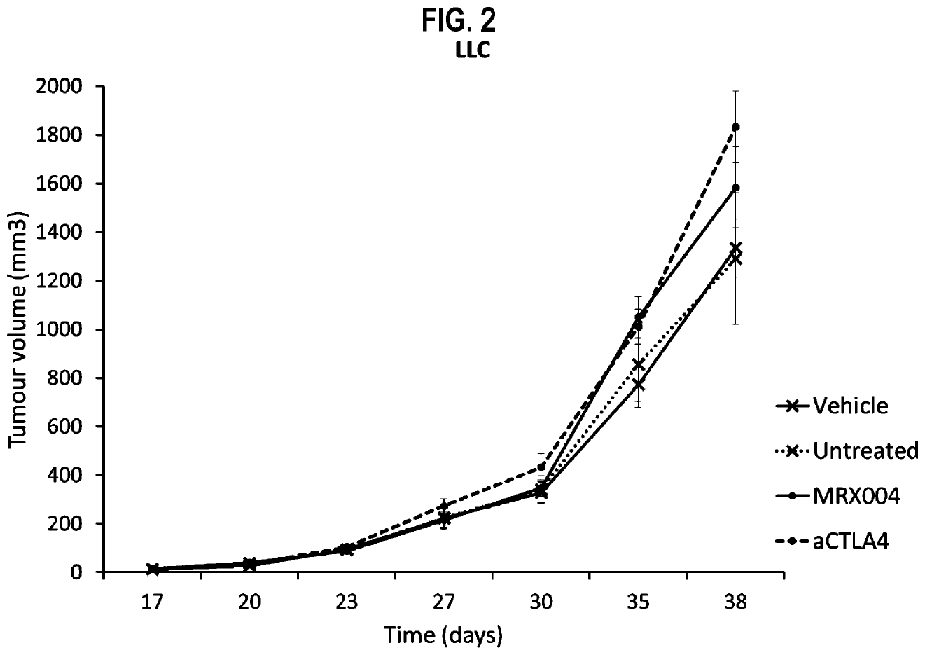

The results are shown in FIG. 2. The negative and positive controls do not appear as would be expected, because tumour volume was greater in the mice treated with the positive control than in the negative control groups. Nevertheless, tumour volume in the mice treated with the bacterial strain of the invention was comparable to the positive control group, which is consistent with a useful therapeutic and immunostimulatory effect.

Antitumor Activity, Hepal-6 Model

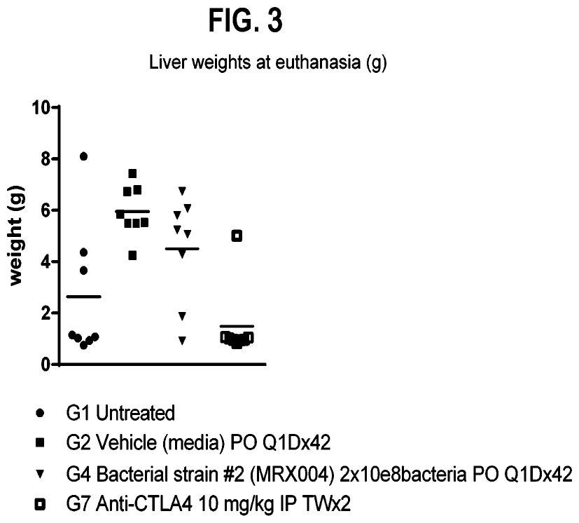

The results are shown in FIG. 3. The untreated negative control does not appear as would be expected, because liver weight was lower in this group than the other groups. However, the vehicle negative control and the positive control groups both appear as would be expected, because mice treated with vehicle alone had larger livers than mice treated with anti-CTLA4 antibodies, reflecting a greater tumour burden in the vehicle negative control group. Treatment with the bacterial strain of the invention led to a clear reduction in liver weight (and therefore tumour burden) relative to the mice in the vehicle negative control group.

These data demonstrate that MRX004 is effective for treating cancer and, in light of the data in Examples 1 and 2, these data support that strain MRX004 may be useful for treating or preventing other diseases associated with reduced immune system activity.

### Example 4—Characterisation of Enzymatic Activity

The Analytical Profile Index (API®) test system consists of strips that contain miniaturised biochemical tests that assay for enzymatic activity in bacterial species. MRX004 (the bacterium deposited under accession number NCIMB 42380) was characterised using two API test systems: Rapid ID 32A—This system is designed specifically for anaerobic species and encompasses tests for carbohydrate, amino acid and nitrate metabolism as well as alkaline phosphatase activity; and API® 50 CH—This system tests for the fermentation of 49 carbohydrate sources, and can be utilised in conjunction with API® CHL Medium for analysis of anaerobic species.

Rapid ID 32A testing was carried out on bacterial colonies as per manufacturer's instructions. Briefly, bacteria were cultured on YCFA agar for 24 hours at 37° C. in an anaerobic workstation. Colonies were removed from plates using a sterile 5 μl inoculating loop and resuspended in a 2 ml ampoule of API® Suspension Medium until a density roughly equivalent to that of McFarland standard No. 4 was achieved. Fifty-five microlitres of bacterial suspension was added to each cupule on a Rapid ID 32A strip, and the urease test was overlayed with two drops of mineral oil. Strips were covered with a plastic lid and incubated aerobically at 37° C. for 4 hours, following which the bottom row of cupules were developed using the following reagents: NIT: 1 drop each of NIT1 and NIT2; IND: 1 drop of James reagent; all remaining cupules: 1 drop of FastBlue reagent. Strips were incubated at room temperature for 5 minutes, following which the colour of each cupule was recorded and assigned a value of negative, intermediate positive or positive.

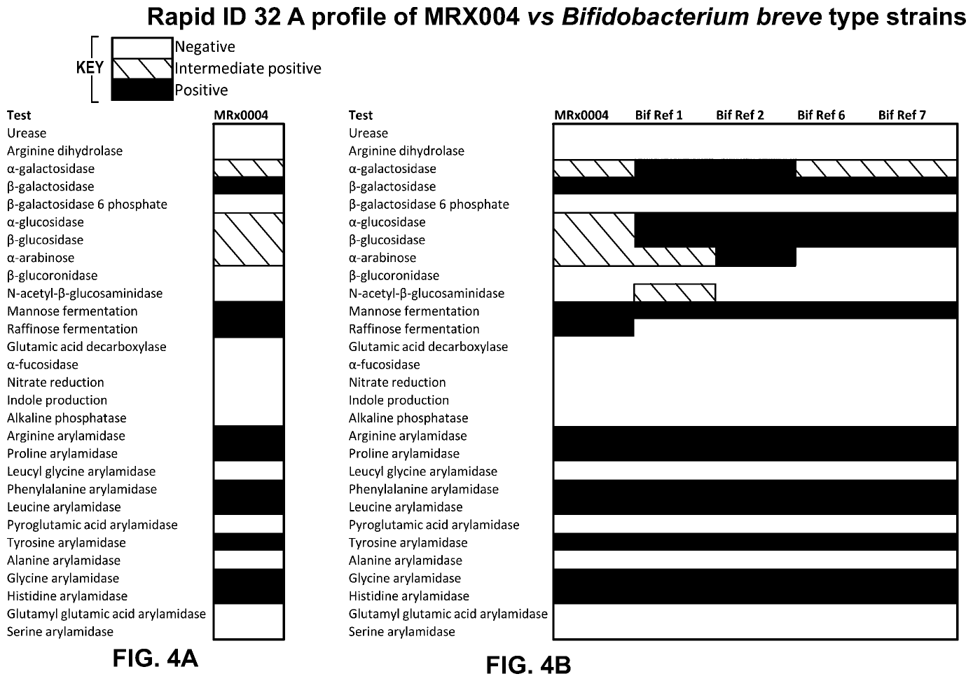

The results of the Rapid ID 32A analysis are shown in FIG. 4. MRX004 tested positive for fermentation of several carbohydrate sources, namely α-galactosidase and β-galactosidase, α-glucosidase and β-glucosidase, α-arabinose, mannose and raffinose, as well as the amino acids arginine, proline, phenylalanine, leucine, tyrosine, glycine and histidine.

Comparative Rapid ID 32A analysis was carried out between MRX004 and four B. breve type strains, which are annotated in FIG. 4B as Bif Ref 1 (DSM 20091), Bif Ref 2 (DSM 20213), Bif Ref 6 (JCM 7017) and Bif Ref 7 (UCC2003). This analysis demonstrated that MRX004 was the only strain tested to ferment the polysaccharide raffinose, which may be significant, because raffinose is involved in the production of bacterial components such as exopolysaccharides, and raffinose fermentation can also reportedly confer effects on the host such as increased caecal butyrate, increased gastrointestinal proliferation and weight loss.

API® 50 CH testing was carried out to further examine carbohydrate metabolism in MRX004. As per manufacturer's instructions, bacteria were cultured in 10 ml YCFA broth for 16-18 hours at 37° C. in an anaerobic workstation. This culture was diluted in 10 ml API® CHL Medium so as to achieve a density roughly equivalent to McFarland standard No. 2, and 110 μl of this mixture was used to inoculate each cupule on a set of API® 50 CH test strips. Test strips were incubated in a humidified incubation box at 37° C. in an anaerobic workstation for 48 hours, following which the colour of each cupule was recorded and assigned a value of negative, intermediate positive, positive or doubtful.

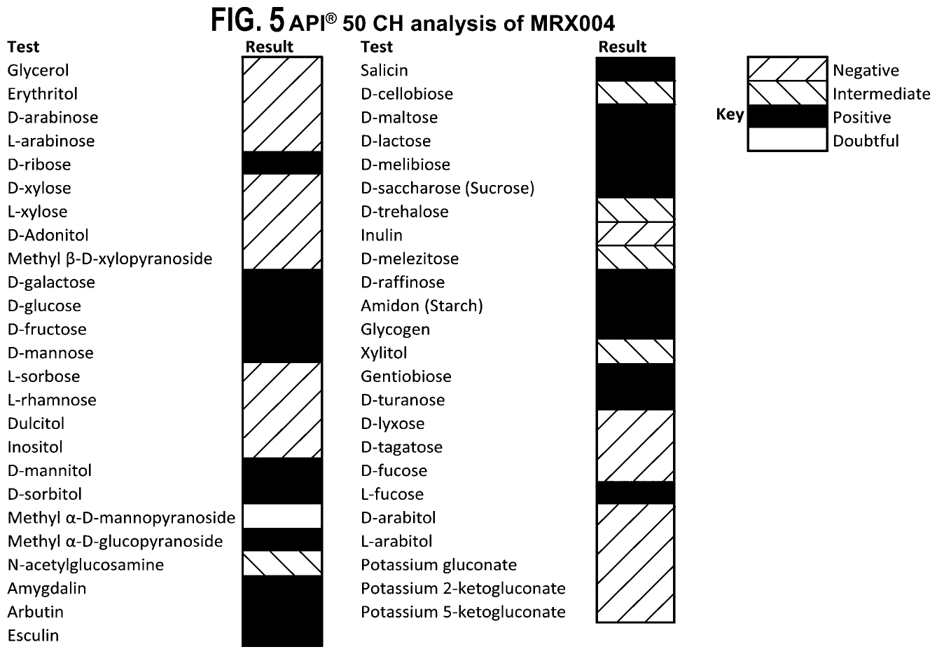

Using API® 50, MRX004 tested positive for utilisation of the following carbohydrate sources: amidon (starch), amygdalin, arbutin, cellobiose, esculin, galactose, gentiobiose, glucose, glycogen, fructose, fucose, lactose, maltose, mannose, mannitol, melibiose, melezitose, methyl a-D-glucopyranoside, N-acetylglucosamine, ribose, saccharose (sucrose), salicin, sorbitol, trehalose, turanose and xylitol (FIG. 5). These results correlated with those obtained for Rapid ID 32A testing in that MRX004 demonstrated fermentation of galactose, glucose, mannose and raffinose in both test systems.

### Example 5—Attachment to Human Cells in YCFA Medium

Summary

The level of binding of strain MRX004 and a number of other Bifidobacterium breve strains to human cells was determined at 3 distinct time points in YCFA medium. The bacteria attached to the human cells were resuspended in medium and the optical density of the medium was then analysed—the higher the optical density, the higher the number of bacterial cells and thus, the higher the level of binding of the bacterial cells to human cells. The MRX004 strain was found to display reduced attachment to human cells compared to the Bifidobacterium breve reference strains.

Results and Analysis

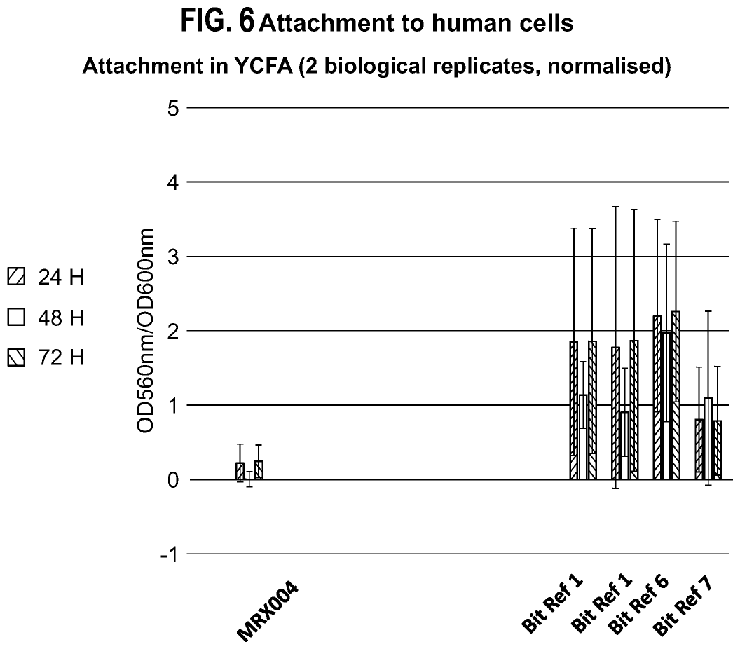

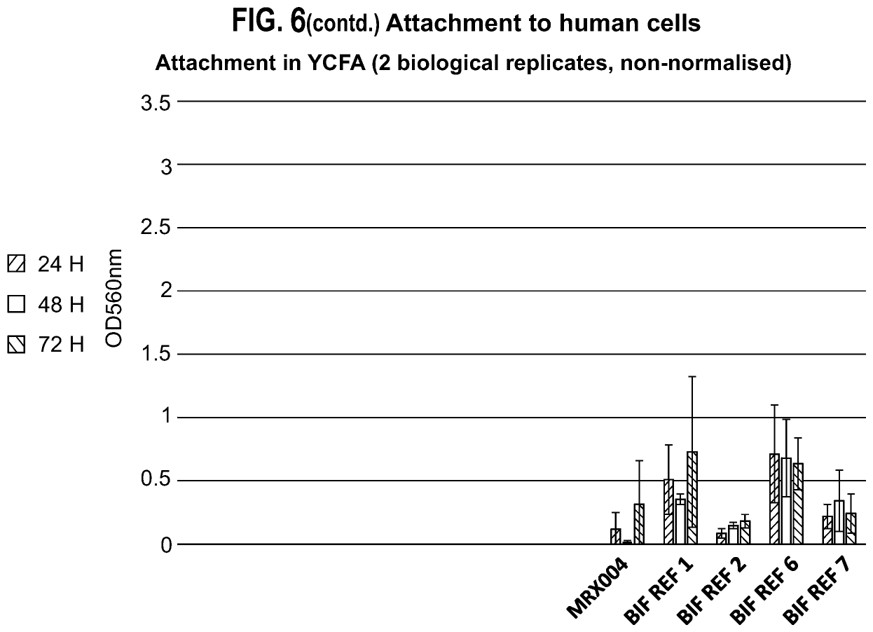

The results of the experiment are shown in FIG. 6.

As shown in FIG. 6, the reference Bifidobacterium breve strains show a high level of attachment to human cells at all time points. On the other hand, the MRX004 strain has a drastically reduced level of attachment to human cells. Therefore, the low adherence to human cells of strain MRX004 may increase the beneficial effect of the compositions of the invention on the immune system.

### Example 6—Stability Testing

A composition described herein containing at least one bacterial strain described herein is stored in a sealed container at 25° C. or 4° C. and the container is placed in an atmosphere having 30%, 40%, 50%, 60%, 70%, 75%, 80%, 90% or 95% relative humidity. After 1 month, 2 months, 3 months, 6 months, 1 year, 1.5 years, 2 years, 2.5 years or 3 years, at least 50%, 60%, 70%, 80% or 90% of the bacterial strain shall remain as measured in colony forming units determined by standard protocols.

### Example 7—Antimicrobial Activity

Introduction

The aim of this experiment was to test the antimicrobial activity potential of several B. breve strains derived from human infants against various indicator strains and to assess whether they produce bacteriocins in vitro.

Methods

A panel of strains were chosen as indicator strains (Table 5), which included closely-related Gram-positive, other Gram-positive and Gram-negative bacteria that have been previously shown to be inhibited by Bifidobacterium species.

Co-culture Assay

Strains were grown for 16 h at 37° C. (30° C. for B. subtilis) under anaerobic conditions (aerobic conditions for E. coli, B. subtilis and S. aureus) from Research Cell Bank (for B. breve test and reference strains) or from bead stocks (for the indicator strains). A lawn of indicator strain was made on a YCFA plate (E&O Labs, UK), left to dry and 10 μl of test strain culture were spotted on top of the lawn. Plates were incubated for 48 h at 37° C. under anaerobic conditions (24 h at 37° C. under anaerobic conditions followed by 24 h at 37° C. under aerobic conditions for E. coli, B. subtilis and S. aureus). Each assay was performed in triplicate (except in duplicate for Bacillus subtilis NCIMB8045 with MRx0004, Test 1, Test 2, Test 3, Test 4, Test 5, Test 6, Test 7 and Test 8, and for Bifidobacterium breve DSM20213, Lactobacillus plantarum NCIMB8826, Clostridium sporogenes ATCC3584, and Staphylococcus aureus NCIMB9518 with all B. breve strains).

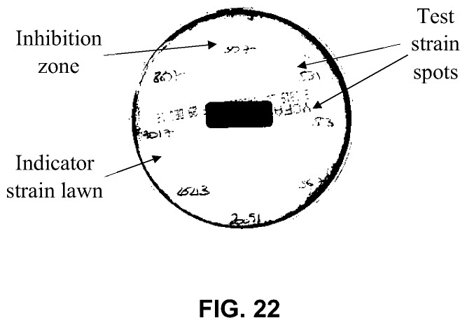

Antimicrobial activity was assessed by measuring the width of the observed inhibition zone, a clear zone around the test strain spot (FIG. 22). A score between 0 and 3 was given to each strain for each biological replicate.

Culture Supernatant

The agar diffusion method was used to test the antimicrobial potential of culture supernatants. In brief, 100 μl of filtered cell-free supernatant were spotted on YCFA agar, pre-inoculated with a lawn of an indicator strain (as described above), into a well punched into the agar. The plate was left to stand for 1 h to allow diffusion and was then incubated for 48 h at 37° C. under anaerobic conditions (aerobic conditions for E. coli, B. subtilis and S. aureus). Three biological replicates were performed.

Results

Co-Culture

Most B. breve strains tested exhibited antagonism activity against E. coli, K pneumoniae, S. Typhimurium and B. subtilis (Table 6). B. breve DSM 20091 was the only strain tested that inhibited the growth of B. breve DSM20213. MRx0004 and other test B. breve strains displayed antimicrobial activity against E. coli, K pneumoniae, S. Typhimurium and B. subtilis (Table 6), with overall higher inhibition observed than with the B. breve reference stains. MRx0004, Test 1, Test 2, Test 3, Test 7, Test 8, Test 11 and Test 12 exhibited particularly potent antimicrobial activity. No antagonistic activity was detected against S. aureus, C. sporogenes and L. plantarum in the conditions tested.

These data indicate that MRX0004 and the related test strains may be useful for treating bacterial infections.

Culture Supernatant

The antimicrobial activity of cell-free supernatants was tested against the same panel of indicator strains. No inhibition was observed for all B. breve strains tested against any of the indicator strains (data not shown, n=3). This suggests that the inhibition observed in the co-culture assays was not due to the secretion of antimicrobial molecules.

### Example 8—Further Characterisation of Enzymatic Activity

The API 32A test system described in Example 4 was used to characterise the B. breve strains tested in Example 7. Rapid ID 32A testing was carried out on bacterial colonies as per manufacturer's instructions. Briefly, bacteria were cultured on YCFA agar for 24 hours at 37° C. in an anaerobic workstation. Colonies were removed from plates using a sterile 5 μl inoculating loop and resuspended in a 2 ml ampoule of API® Suspension Medium until a density roughly equivalent to that of McFarland standard No. 4 was achieved. Fifty-five microlitres of bacterial suspension was added to each cupule on a Rapid ID 32A strip, and the urease test was overlayed with two drops of mineral oil. Strips were covered with a plastic lid and incubated aerobically at 37° C. for 4 hours, following which the bottom row of cupules were developed using the following reagents: NIT: 1 drop each of NIT1 and NIT2; IND: 1 drop of James reagent; all remaining cupules: 1 drop of FastBlue reagent. Strips were incubated at room temperature for 5 minutes, following which the colour of each cupule was recorded and assigned a value of negative, intermediate positive or positive.

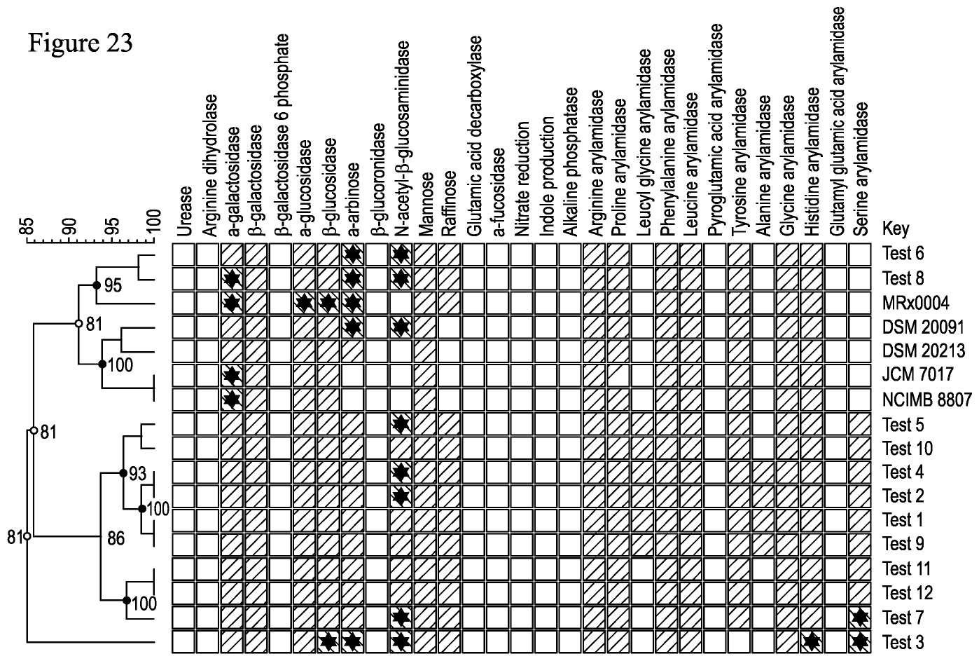

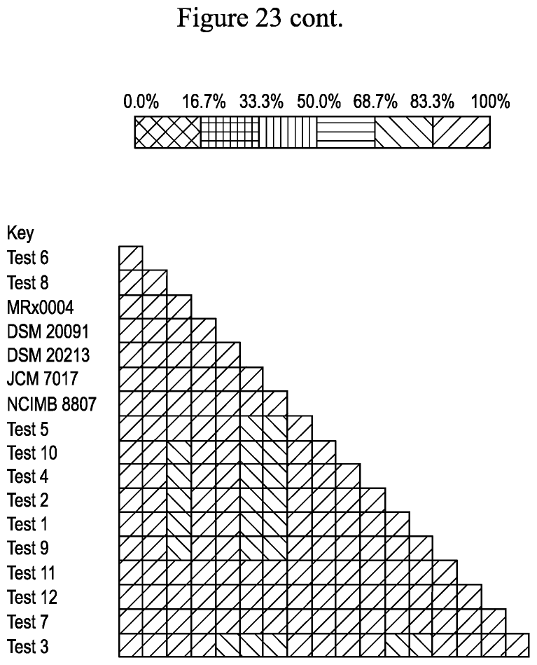

The results of the Rapid ID 32A analysis are shown in FIG. 23. As found in Example 4,MRX004 tested positive for fermentation of several carbohydrate sources, namely α-galactosidase and β-galactosidase, α-glucosidase and β-glucosidase, α-arabinose, mannose and raffinose, as well as the amino acids arginine, proline, phenylalanine, leucine, tyrosine, glycine and histidine.

Rapid ID 32A analysis was also performed on the other test B. breve strains (Test 1-Test 12) and the B. breve reference strains (DSM 20091, DSM 20213, JCM 7017, and NCIMB 8807/UCC2003) that were studied in Example 7. The test strains generally showed greater antimicrobial activity than the reference strains and showed metabolism patterns with similarity to MRX004.

Interestingly, MRX004 and the test strains ferment the polysaccharide raffinose, whilst the four reference strains do not. As noted above, raffinose is involved in the production of bacterial components such as exopolysaccharides.

Also, MRX004 and Test 3 both have potent anti-microbial activity and both exhibit intermediate fermentation of β-glucosidase and α-arabinose.

MRX004 and Test 2 both have potent anti-microbial activity and neither exhibits positive fermentation of N-acetyl-β-glucosaminidase.

MRX004 and Test 8 both have potent anti-microbial activity and both exhibit intermediate fermentation of α-galactosidase and α-arabinose.

Test 11 and Test 12 both have potent anti-microbial activity and both ferment serine arylamidase but not leucyl glycine arylamidase and not alanine arylamidase.

Test 3 and Test 7 both have potent anti-microbial activity and both exhibit intermediate fermentation of serine arylamidase.

### Example 9—Pulsed-Field Gel Electrophoresis

Pulsed-field Gel Electrophoresis (PFGE) was used to characterise the B. breve strains tested in Example 7. The results are shown in FIG. 24. The test B. breve strains, which exhibited greater anti-microbial activity than the reference B. breve strains, were found to be grouped together with similar patterns and can be distinguished from the reference strains.

### Example 10—Pulsed-Field Gel Electrophoresis

A further pulsed-field gel electrophoresis (PFGE) study was performed on MRx004 and the reference B. breve strains. PFGE is routinely applied as the “gold standard” for strain typing in clinical laboratories [100] and has been reported to be an effective method for discriminating human fecal Bifidobacterium isolates [101]. Indeed, many studies have explored the relationships of Bifidobacterium isolates using fingerprint analysis of PFGE-resolved fragments of genomic DNA, digested with either Xbal or Spel restriction enzymes [102,103]. Spel has proven particularly useful in plasmid profiling and intraspecific genotyping of Bifidobacterium breve [104,105].

PFGE Plug Preparations

Agarose gel plugs of high molecular weight DNA for PFGE were prepared according to a published protocol [106].

Restriction of PFGE Plugs

Single slices (2 mm×2 mm) were washed three times for 15 min in 1 ml 10 mM Tris.Cl, 0.1 mM EDTA (pH 8.0) at room temperature. Each slice was pre-incubated with 250 μl of restriction buffer recommended for the enzyme for 30 min at 4° C. and then replaced with 250 μl of fresh buffer containing 20 units of restriction enzyme Smal. Restriction digests were carried out overnight at 25° C, as recommended by the supplier (New England Biolabs (UK) Ltd).

PFGE

Treated (restriction enzyme) and untreated plugs of genomic DNA (gDNA) were examined under the following conditions. The γ ladder was heated at 45 C prior to loading it into the gel. Running conditions were 6.0 V/cm at 14° C. for 20 h with pulse times ramped from 1 to 20 s in 0.5×TBE buffer. A lambda DNA ladder (Bio-Rad) was used as the size marker. The plugs were placed in wells of 1.0% agarose gels (Bio-Rad) made with 0.5×TBE (1 M Tris-borate, 0.5 M EDTA, pH 8.5), sealing with the same agarose. DNA fragments were resolved in 0.5×TBE running buffer maintained at 14° C. using a CHEF-DR III pulsed-field system (Bio-Rad Laboratories, Hercules, Calif.) at 6 V/cm for 18 h. Linear ramped pulse times of were selected. A linear ramped pulse times of 1 s-15 s were employed for separation of the fragments. Gels were stained in distilled water containing 0.5 μg/ml ethidium bromide for 120 min under light-limited conditions.

PFGE Banding Pattern Analysis

Banding patterns were manually assessed using the guidelines outlined by [107]. The PFGE image was processed in BioNumerics 7.6 (Applied Maths) to generate a band fingerprint for each strain. Cluster analysis on fingerprints was carried out using the Jaccard similarity coefficient (recommended for fingerprint-type analysis) and Unweighted Pair Group Method with Arithmetic Mean (UPGMA).

Results

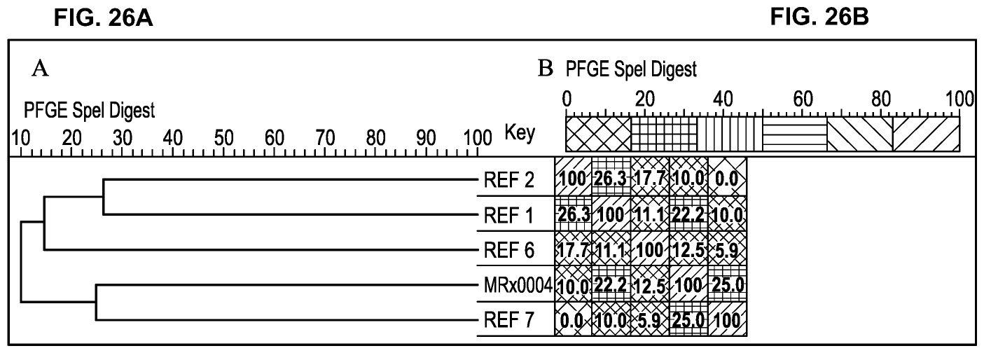

The DNA digestion and electrophoretic conditions employed in this study have previously been shown to be effective for subtyping species of B. breve [104] and were found to provide sufficient resolution (more than 10 observable bands) to distinguish strains of B. breve at a subspecies level. The restriction fragments of 4/5 strains were distinct and were well resolved (FIG. 25). Cluster analysis (FIG. 26) suggests that the genotype of strain MRx0004 is more closely related to that of B. breve REFI than the other B. breve strains analysed in this study.

### Example 11—T Cell Differentiation

The ability of MRx0004 to induce T-cell differentiation was explored in vitro on peripheral blood mononuclear cells (PBMCs, Stemcell, Cat:70025). Briefly, PBMCs were plated in 96-well plates plated with anti-CD3 (Ebioscience, CD3 monoclonal antibody (OKT3 clone), functional grade, cat. No. 16-0037-81) at 400,000/well in 50 μcRPMI medium per well (cRPMI contains RPMI 1640 (+L-Glut, 21875-034) 2mM final conc. Stock 200 mM.; 10% HI FBS (Gibco life technologies, 10082-147); 50 μm mercaptoethanol (Gibco life technologies, 21985-023); and 1% pen/strep (P4333, 10 mg/m1). Heat-killed MRx0004 (prepared by incubation at 80° C. for 30 minutes, after which the cultures were washed with PBS and resuspended in appropriate cell culture medium and viable counts were confirmed by plating) was then added to each well, 4,000,000 in 100 μl/well. Following 3 days in a 37° C. incubator, the cells were removed and re-suspended in a medium containing PMA-(Sigma, Cat no. P8139), Ionomycin (Sigma, Cat no. 13909) and GolgiSTOP (BD, Cat no 554724) for 5 hours. PMA stock was 1 mg/ml in DMSO which was further diluted in 100 μg/ml (each sample required 50 ng/ml in cRPMI), Ionomycin stock was 1 mM in DMSO (1 μM in cRPMI was used) and GolgiStop concentration was used at 4μl/6ml. Supernatants were passed through a 0.22 μm filter and diluted appropriately in co-culture medium.

The cells were then subjected to a flow cytometry staining:

After washing, the cells were incubated with viability dye (Viobility 405/520 Fixable Dye from Miltenyi biotec, 1 μl/sample)+human Fc block, cat. 564219 (1 μl/sample) in PBS for 10 mins in the dark at room temperature. The surface antibodies (2 μl of each) were then added directly to the wells for 10 mins in the dark at room temperature—CD3-APC-Vio 770 (Miltenyi, cat. No. 130-113-136), CD4-VioBlue (Miltenyi, cat. No. 130-114-534) and CD25-VioBright FITC (Miltenyi, cat. No. 130-113-283). The cells were then washed twice in PBS and spun down at 300g/5min/RT.

The eBioscience FoxP3 transcription factor staining buffer was then used to fix and permeabilise the cells (cat. No. 00-5523). Following the eBioscience protocol, a perm/fix buffer was prepared using 1× concentrate and 3 diluent. The cells were fixed for 1 h at RT and then washed 2× in 1× Perm wash and spun down at 300g/5min/RT. The following intracellular staining or transcription factor antibodies were added to the samples in perm wash (1×) for 45mis/dark/RT or in the fridge overnight (up to 18 h), followed by washing the antibodies 2× using Perm wash (300 μl) and re-suspension in PBS (250 μl) to acquire on the cytometer:

| Intracellular markers | Transcription factors |
|---|---|
| 2 ul IL10-PE | 5.5 ul FoxP3-PE-Cy7 |
| 2 ul IFNy-PE Vio770 | 9 ul Tbet-APC |
| 10 ul IL17a-APC | 9 ul RoRyt-PE |

Anti IFNy-PE Vio770 human antibodies (Miltenyi, cat. No. 130-114-025)
Anti IL10-PE human antibodies (Miltenyi, cat. No. 130-112-728)
Anti IL17a-APC human antibodies (Miltenyi, cat. No. 130-099-202)
Anti RoRyt-PE human antibodies (Miltenyi, cat. No. 130-103-837)
Anti Tbet-APC human antibodies (Miltenyi, cat. No. 130-098-655
Foxp3 monoclonal antibody (236A/E7), Pe cy7 (ebioscience) cat. No. 25-4777-41

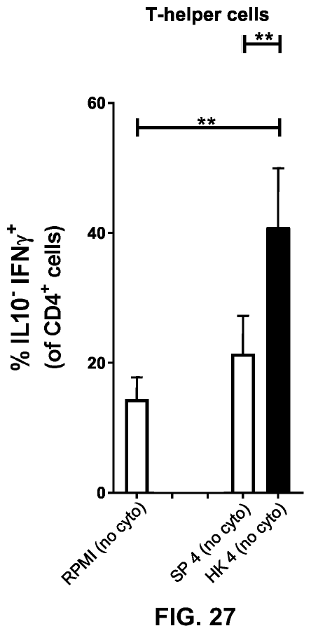

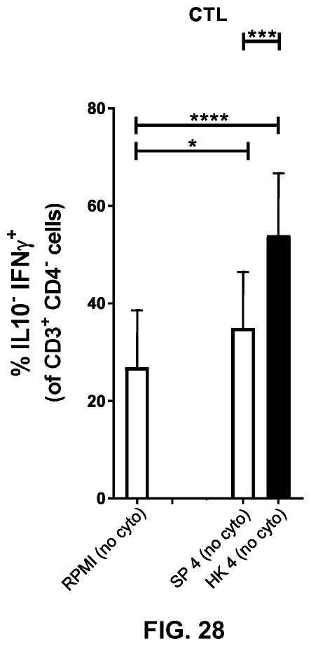

As can be seen in FIGS. 27 and 28 both supernatant of MRx0004 (SP 4) and heat-killed MRx0004 (HK 4) were able to induce differentiation of T helper cells and cytotoxic T cells, respectively, even in the absence of cytokines to induce differentiation (no cyto).

Tables

TABLE 1
Strains, plasmids and primers used strain generation in this study

| | | |
|---|---|---|
| Strain | Description | Reference |

B. breve strains

| | | |
|---|---|---|
| MRx0004 | B. breve human isolate | This study |
| MRx0004 | MRx0004 pGTF insertion mutant | This study |
| pGTF::pORI19 | | |
| MRx0004 | MRx0004 pGTF insertion mutant harbouring the empty | This study |
| pGTF::pORI19 | pBC1.2 vector | |
| pBC1.2 | | |
| MRx0004 | MRx0004 pGTF insertion mutant complemented | This study |
| pGTF::pORI19 | | |
| pGTF-pBC1.2 | | |

E. coli strains

| | | |
|---|---|---|
| X11 Blue | Cloning host, tet^{r} | Stratgene, La |
| | | Jolla, CA, USA |
| EC101 | Cloning host, repA⁺ km^{r} | [108] |
| EC101 pWSK29- | E. coli strain for in vitro methylation by MRx0004 Type I | This study |
| MRx-M + S | methylase | |

Plasmids

| | | |
|---|---|---|
| pORI19 | Lactococcal ORI⁺ RepA⁻ expression vector | [108] |
| pWSK29 | Amp^{r}, low copy mumber E. coli cloning plasmid | [109] |
| pNZ8048 | Cm^{r}, nisin inducible translational fusion factor | [110] |
| pAM5 | pBC1-pUC19-Tc^{r} | [111] |
| pPKCM | pblueCm harbouring rep pCIBA089 | [112] |
| pSKEM | pblueEm harbouring rep pCIBA089 | [112] |
| pNZEM | Em^{r}, nisin inducible | Margolles, |
| | | unpublished |
| pDM1 | pAM5 derivative containing spectinomycin resistance | [113] |
| | cassette | |
| pDM2 | pDG7 derivative containing spectinomycin resistance | [113] |
| | cassette | |

Primers

| Primer Name | Sequence (5′-3′) | Reference |
|---|---|---|
| PWSK_MRxM + S | CGTCCGTCTAGAATAAGGAGGCACTCACCATGA | This study |
| FxbaI | ATAAGCAGCAGCTTGC (SEQ ID NO: 2) | |
| PWSK_MRxM + S | GCTCTACTCGAGGCGATATGAGGCGAGCTTCACG | This study |
| FxhoI | (SEQ ID NO: 3) | |
| TetWconfirm F2 | CAGGCATTGAAGGAATCG (SEQ ID NO: 4) | This study |
| TetWconfirm F2 + | CTGGCTAAGCTTGTGCATGGGCTCAGTCCTTC | This study |
| MRx-pGTF-comp | (SEQ ID NO: 5) | |

TABLE 2
List of the 20 proteins with the highest peptide spectrum match (PSM) values identified
in MRx0004 late log culture supernatants as identified by nanoLC-MS/MS.
This table lists all proteins with a PSM value ≥5 identified in three biological replicates.

| | Normalised | | | Predicted | | | |
|---|---|---|---|---|---|---|---|
| | PSM value ^{a} | | Functional | localization | | MW | |
| Annotation | Average | SD | category ^{b} | (score) ^{c} | | (kDa) ^{d} | pI ^{d} |
| Pullulanase | 351.33 | 33.62 | Carbohydrates | E | (9.98) | 181.9 | 4.77 |
| NlpC/P60 family protein | 82.00 | 15.62 | n.a. | E | (9.72) | 33.5 | 8.18 |
| NlpC/P60 family protein | 71.67 | 13.61 | n.a. | n.d. | (3.33) | 24.9 | 6.18 |
| Probable solute binding protein of | 56.00 | 7.00 | n.a. | n.d. | (3.33) | 32.8 | 4.69 |
| ABC transporter system | | | | | | | |
| Cell division protein FtsI | 56.00 | 4.36 | Cell Division | CM | (9.68) | 63.4 | 5.39 |
| | | | and Cell Cycle | | | | |
| Multiple sugar ABC transporter, | 54.67 | 4.16 | Carbohydrates | n.d. | (3.33) | 49.3 | 5.01 |
| substrate-binding protein | | | | | | | |
| Xylulose-5-phosphate | 45.33 | 7.02 | n.a. | n.d. | (2.5) | 92.3 | 5.26 |
| phosphoketolase | | | | | | | |
| Methionine ABC transporter | 38.67 | 2.08 | Amino Acids | CM | (9.51) | 35.2 | 5.17 |
| substrate-binding protein | | | and Derivatives | | | | |
| Hypothetical protein | 35.67 | 8.33 | n.a. | n.d. | (2.5) | 34.6 | 7.47 |
| Transaldolase | 32.67 | 3.79 | Carbohydrates | C | (7.5) | 39.7 | 4.97 |
| Maltose/maltodextrin ABC | 30.00 | 6.08 | Carbohydrates | n.d. | (3.33) | 43.9 | 4.48 |
| transporter | | | | | | | |
| NAD-dependent glyceraldehyde-3- | 30.00 | 3.61 | Carbohydrates | C | (9.97) | 37.8 | 5.44 |
| phosphate dehydrogenase | | | | | | | |
| Hypothetical protein | 29.67 | 4.93 | n.a. | CW | (9.21) | 47.1 | 5.48 |
| Solute binding protein of ABC | 26.67 | 1.15 | n.a. | CW | (9.2) | 58.7 | 5.45 |
| transporter system for peptides | | | | | | | |
| Dipeptide-binding ABC | 24.00 | 4.58 | Membrane | n.d. | (5.13) | 59.1 | 5.31 |
| transporter, periplasmic substrate- | | | Transport | | | | |
| binding component | | | | | | | |
| NlpC/P60 family protein | 21.00 | 1.00 | n.a. | E | (9.73) | 24.5 | 6.30 |
| FKBP-type peptidyl-prolyl cis-trans | 20.00 | 2.65 | Potassium | n.d. | (5.15) | 33.4 | 5.83 |
| isomerase FkpA precursor | | | metabolism | | | | |
| Glucose-6-phosphate isomerase | 18.67 | 3.21 | Carbohydrates | C | (9.97) | 62.9 | 4.97 |
| Multimodular transpeptidase- | 18.00 | 2.65 | Cell Wall and | CM | (10) | 81.9 | 5.72 |
| transglycosylase | | | Capsule | | | | |
| Chaperone protein DnaK | 17.67 | 3.21 | Protein | C | (9.97) | 66.9 | 4.87 |
| | | | Metabolism | | | | |

^{a} Average peptide spectrum match values from 3 biological replicates normalized to 1 × 10⁹ cfu in and corresponding standard deviation values.
^{b} Subsystem category distribution as annotated by RAST [114, 115]. n.a. not assigned.
^{c} Cellular localization as predicted using PSORTb v3.0 (Yu et al., 2010a). C: cytoplasmic, CM: cytoplasmic membrane, CW: cell wall, E: extracellular, n.d.: not determined.
^{d} Parameters calculated by Proteome Discoverer (Thermo Scientific, Waltham, MA, USA). MW: molecular weight, pI: isoelectric point.

TABLE 3
List of the top 20 proteins with the highest peptide spectrum match (PSM)
value identified in MRx0004 cell shavings as identified by nanoLC-MS/MS.
This table lists the 62 proteins identified in both MRx0004 shaved and shed protein
fractions and the 44 proteins identified exclusively in MRx0004 shaved protein fraction.
Proteins listed were detected at PSM value ≥5 and in three biological replicates.

| | Shaved proteins | | Shed proteins | | | Predicted | |
|---|---|---|---|---|---|---|---|
| | Average | | Average | | Functional | localization | |
| Annotation | PSM ^{a} | SD | PSM ^{a} | SD | category ^{b} | (score) ^{c} | |
| Pullulanase | 136.67 | 17.47 | 79.00 | 10.54 | Carbohydrates | E | (9.98) |
| Type I polyketide synthase | 54.67 | 10.69 | 11.67 | 6.43 | Unassigned | CM | (9.78) |
| Xylulose-5-phosphate | 49.67 | 2.89 | 52.00 | 9.17 | Unassigned | n.a. | (2.5) |
| phosphoketolase | | | | | | | |
| Cell division protein FtsI | 34.33 | 9.24 | 33.00 | 10.00 | Cell Division | CM | (9.68) |
| | | | | | and Cell Cycle | | |
| Glucose-6-phosphate isomerase | 32.33 | 2.31 | 30.33 | 5.86 | Carbohydrates | C | (9.97) |
| Translation elongation factor Tu | 32.00 | 5.00 | 24.33 | 8.39 | Protein | C | (9.97) |
| | | | | | Metabolism | | |
| Maltose/maltodextrin ABC | 32.00 | 7.94 | 18.67 | 5.13 | Carbohydrates | n.a. | (3.33) |
| transporter, substrate binding | | | | | | | |
| periplasmic protein MalE | | | | | | | |
| Glycogen phosphorylase | 31.00 | 3.00 | 34.33 | 5.69 | Carbohydrates | C | (7.5) |
| Transaldolase | 31.00 | 2.65 | 33.33 | 2.08 | Carbohydrates | C | (7.5) |
| Multiple sugar ABC transporter, | 30.00 | 6.93 | 36.00 | 11.79 | Carbohydrates | n.a. | (3.33) |
| substrate-binding protein | | | | | | | |
| Pyruvate formate-lyase | 26.67 | 8.33 | 21.67 | 7.77 | Carbohydrates | C | (9.97) |
| Solute binding protein of ABC | 26.00 | 0.00 | 12.67 | 2.89 | Unassigned | CW | (9.2) |
| transporter systemy for peptides | | | | | | | |
| Transketolase | 24.00 | 6.08 | 23.67 | 3.79 | Carbohydrates | C | (7.5) |
| Methionine ABC transporter | 23.33 | 2.52 | 22.33 | 4.04 | Amino Acids | CM | (9.51) |
| substrate-binding protein | | | | | and Derivatives | | |
| Translation elongation factor G | 23.00 | 13.89 | 15.67 | 8.02 | Protein | C | (9.97) |
| | | | | | Metabolism | | |
| Polyribonucleotide | 22.67 | 5.03 | 12.33 | 5.69 | Unassigned | C | (9.97) |
| nucleotidyltransferase | | | | | | | |
| PTS system, beta-glucoside-specific | 21.67 | 4.62 | 6.67 | 2.89 | Unassigned | CM | (10) |
| IIB component | | | | | | | |
| FKBP-type peptidyl-prolyl cis-trans | 21.33 | 5.86 | 7.67 | 3.06 | Potassium | n.a. | (5.15) |
| isomerase FkpA precursor | | | | | metabolism | | |
| Chaperone protein DnaK | 21.00 | 6.24 | 10.00 | 3.61 | Protein | C | (9.97) |
| | | | | | Metabolism | | |
| Probable solute binding protein of | 20.33 | 4.04 | 19.67 | 2.08 | Unassigned | n.a. | (3.33) |
| ABC transporter system | | | | | | | |

^{a} Average peptide spectrum match values from 3 biological replicates and corresponding standard deviation values, n.d.: not detected.
^{b} Subsystem category distribution as annotated by RAST [116, 114]. n.a. not assigned.
^{c} Cellular localization as predicted using PSORTb v3.0 [117]. C: cytoplasmic, CM: cytoplasmic membrane, CW: cell wall, E: extracellular, n.a.: not assigned.

TABLE 4
Comparison of host-interaction proteins identified in
MRx0004 and EPS^{neg} strains shaved protein fractions.
This table lists selected proteins, identified by nanoLC-MS/MS in the shaved
protein fractions, in three biological replicates and with a PSM value ≥ 5.

| | MRx0004^{a} | | EPS^{neg a} | | |
|---|---|---|---|---|---|
| | Average | | Average | | |
| | normalised | | normalised | | |
| Description | PSM | SD | PSM | SD | Functional category ^{b} |
| Type I polyketide synthase | 28.04 | 4.47 | 57.06 | 2.17 | n.a. |
| Transaldolase | 17.33 | 1.44 | 24.70 | 0.60 | Carbohydrates |
| Transketolase | 14.18 | 2.50 | 25.40 | 0.60 | Carbohydrates |
| Translation elongation factor Tu | 13.23 | 1.64 | 26.10 | 4.55 | Protein Metabolism |
| Chaperone protein DnaK | 12.29 | 4.33 | 21.92 | 2.09 | Protein Metabolism |
| NAD-dependent glyceraldehyde-3- | 11.97 | 3.32 | 16.35 | 1.59 | Carbohydrates |
| phosphate dehydrogenase | | | | | |
| Heat shock protein 60 family | 11.66 | 6.29 | 13.92 | 2.17 | Protein Metabolism |
| chaperone GroEL | | | | | |
| Enolase | 5.04 | 3.04 | 8.70 | 1.59 | Carbohydrates |
| Phosphoglycerate mutase | n.d. | n.d. | 5.22 | 1.04 | Carbohydrates |

^{a}Average peptide spectrum match values, normalized per μg of total protein, from 3 biological replicates and corresponding standard deviation values (SD). n.d: no peptides detected.
^{b} Subsystem category distribution as annotated by RAST [116, 114]. n.a. no subcategory assigned.

TABLE 5
List of indicator strains used in Example 7

| | Gram | |
|---|---|---|
| Strain name | stain | Description |
| Escherichia coli ATCC 11775 | Negative | Human gut commensal |
| Klebsiella pneumoniae NCIMB 10197 | Negative | Human gut commensal, lung pathogen |
| Salmonella Typhimurium NCIMB | Negative | Human gut commensal, opportunistic pathogen |
| 10248 | | |
| Lactobacillus plantarum NCIMB 8826 | Positive | Human gut commensal, closely-related bacteria |
| Clostridium sporogenes ATCC 3584 | Positive | Ubiquitous human gut commensal |
| Bacillus subtilis NCIMB 8045 | Positive | Human gut commensal |
| Staphylococcus aureus NCIMB 9518 | Positive | Human commensal, opportunistic pathogen |
| Bifidobacterium breve DSM20213 | Positive | B. breve type strain |

TABLE 6
Antimicrobial activity of test and reference B. breve strains against a panel of indicator strains (n = 3)

| | | K. | S. | B. | B. | S. | C. | |
|---|---|---|---|---|---|---|---|---|
| | E. coli | pneumoniae | Typhimurium | subtilis | breve | aureus | sporogenes | L. plantarum |
| | ATCC | NCIMB | NCIMB | NCIMB | DSM | NCIBM | ATCC | NCIMB |
| Strain | 11775 | 10197 | 10248 | 8045 | 20213* | 9518* | 3584* | 8826* |
| MRx0004 | 3 | 2 | 2 | 2* | 0 | 0 | 0 | 0 |
| Test 1 | 1 | 2 | 1 | 2* | 0 | 0 | 0 | 0 |
| Test 2 | 2 | 2 | 2 | 2* | 0 | 0 | 0 | 0 |
| Test 3 | 2 | 2 | 2 | 2* | 0 | 0 | 0 | 0 |
| Test 4 | 1 | 2 | 0 | 1* | 0 | 0 | 0 | 0 |
| Test 5 | 1 | 1 | 1 | 1* | 0 | 0 | 0 | 0 |
| Test 6 | 1 | 1 | 1 | 1* | 0 | 0 | 0 | 0 |
| Test 7 | 2 | 2 | 2 | 1* | 0 | 0 | 0 | 0 |
| Test 8 | 2 | 1 | 1 | 2* | 0 | 0 | 0 | 0 |
| Test 9 | 1 | 1 | 1 | 1 | 0 | 0 | 0 | 0 |
| Test 10 | 1 | 1 | 1 | 1 | 0 | 0 | 0 | 0 |
| Test 11 | 2 | 2 | 2 | 2 | 0 | 0 | 0 | 0 |
| Test 12 | 2 | 2 | 2 | 2 | 0 | 0 | 0 | 0 |
| B. breve | 1 | 1 | 1 | 1 | 1 | 0 | 0 | 0 |
| REF1 | | | | | | | | |
| (DSM | | | | | | | | |
| 20091) | | | | | | | | |
| B. breve | 1 | 1 | 0 | 1 | 0 | 0 | 0 | 0 |
| REF2 | | | | | | | | |
| (DSM | | | | | | | | |
| 20213) | | | | | | | | |
| B. breve | 1 | 1 | 1 | 2 | 0 | 0 | 0 | 0 |
| REF6 | | | | | | | | |
| (JCM | | | | | | | | |
| 7017) | | | | | | | | |
| B. breve | 0 | 0 | 0 | 2 | 0 | 0 | 0 | 0 |
| REF7 | | | | | | | | |
| (NCIMB | | | | | | | | |
| 8807/ | | | | | | | | |
| UCC2003) | | | | | | | | |

### Example 12—MRx004 has an Immunostimulatory Effect in the Spleen

Summary

The object of this study was to characterise the in vitro immunostimulatory properties of MRx0004 in the spleen.

Materials and Methods

Treatments: Untreated, 10% YCFA and 10% Bifidobacterium breve strain MRx0004.

Preparation of Splenocytes

Splenocytes were freshly prepared from spleen isolated from female C57BL/6 mice between 6 and 8 weeks old. Briefly, splenocytes were plated at 900,000 cells/well in 96 well plates in RPMI 1640 with 10% FBS, 2 mM L-Glutamine and 100 U/ml penicillin, 100 μg/ml streptomycin, 55 μM of β-mercaptoethanol, resting or stimulates with 10% bacterial media YCFA+ (Blank media) or with 10% cell-free bacterial supernatant from stationary MRx0518 culture and then incubated for 72 h in a CO2 incubator at 37° C. Afterwards cell free supernatants were collected, spun down for 5 minutes at 500 g at 4° C. Samples were then collected and stored at −80° C. for cytokine analysis.

MTT assay

The MTT assay kit was purchased from Merck Millipore (Cat n. CT01). After 72 h incubation, 10 μ1 of MTT solution was added to each well, cells were incubated in CO2 incubator for 4h. Afterwards 100 μ1 of isopropanol/0.04 M HCL solution was added to each well and the absorbance was measured at 560nm wavelength and a reference wavelength of 655 nm.

Cytokine Analysis

Cytokine quantification was conducted using a 26-plex Mouse ProcartaPlex multiplex immunoassay following the manufacturer's recommendations (Thermo Fischer Scientific). Briefly, 50 μl of cell-free co-culture supernatants were used for cytokine quantification using a MAGPIX® MILLIPLEX® system (Merck) with the xPONENT software (Luminex, Austin, Tex., USA). Data was analysed using the MILLIPLEX® analyst software (Merck) using a 5-parameter logistic curve and background subtraction to convert mean fluorescence intensity to pg/ml values.

Flow cytometry

Cells were first stained with the Viobility 405/520 Fixable Dye (Miltenyi Biotec Ltd. Bergisch Gladbach, Germany) to discriminate between live and dead cells for 10 min in the dark at room temperature. They were then stained with a cocktail of antibodies for CD3, CD4, CD8 and IFN-γ to determine cell phenotype (Miltenyi REA antibodies) and incubated for a further 10 min at room temperature. Cells were then washed and resuspended in PBS and immediately analysed via flow cytometric analysis. Isotypes were used for all antibodies during the first experiment to help set gates and FMO controls were included throughout all the experiments. All experiments were performed using a BD FACS Aria II with the stopping gate for acquisition set on 100,000 cells in the “Live” gate using FACSDiva software (BD Biosciences, Reading, UK). The analysis was conducted using Flowjo version 10.4.2 software (FlowJo LLC, Oregon, USA) and was based on live cells identified with the viability dye.

Results

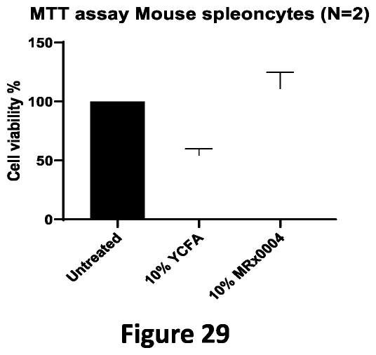

The viability of the splenocytes after treatment was assessed using the MTT assay, which measured their metabolic activity. FIG. 29 shows that splenocytes were viable after treatment with MRx0004.

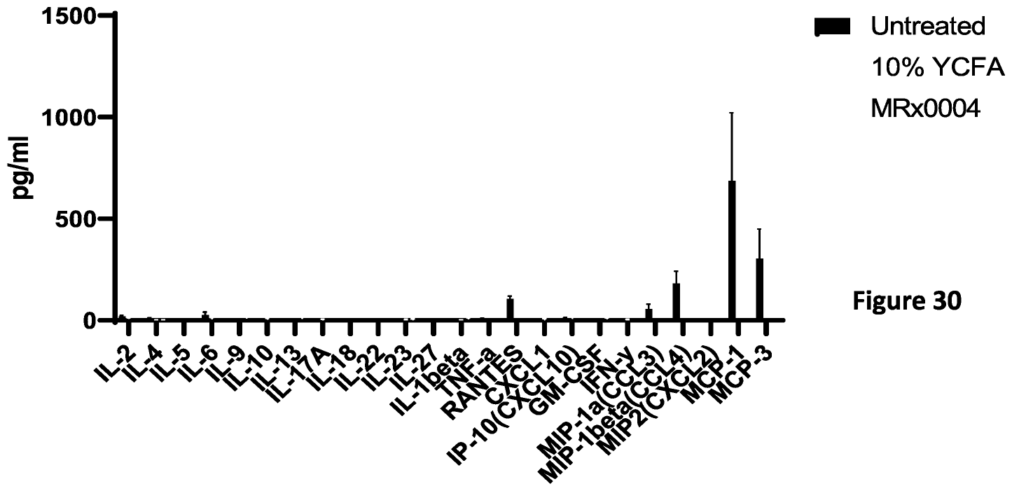

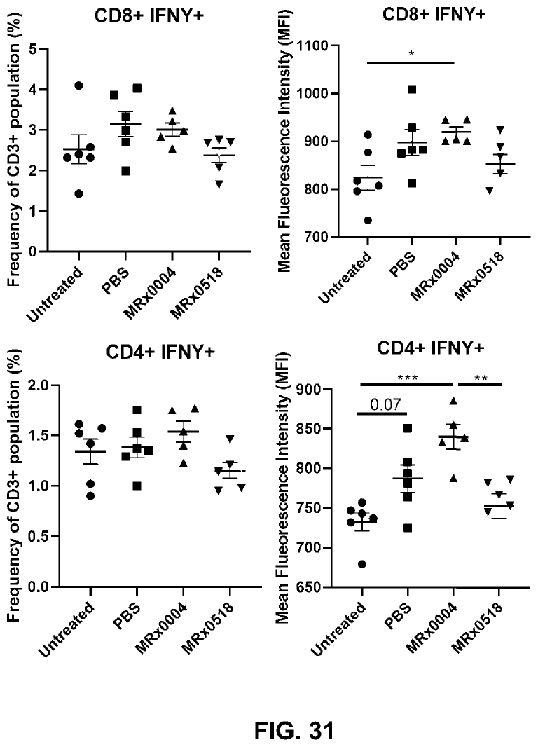

FIG. 30 shows that treatment with MRx0004 led to an increase in a variety of proinflammatory cytokines in the spleen, including IL-6, IL-17α IL-22, TNF-α, RANTES, IFN-γ, CCL3, CCL4 and CXCL2. These data indicate that live Bifidobacterium breve have a stimulatory effect on the immune system. The ability of MRx0004 to activate CD8+and CD4+ T cells to produce IFNγ is shown in FIG. 31.

In combination with the PBMC data discussed in Examples 2 and 11, these data support the ability of bacterial strains from the species Bifidobacterium breve to stimulate the immune system, by activating T-cells and increasing the level of proinflammatory cytokines, in multiple tissues.

Sequences
(consensus 16S rRNA sequence for Bifidobacterium
breve strain deposited under accession number NCIMB
42380)
SEQ ID NO: 1
GGGACAGGCTCAGGATGAACGCCGGCGGCGTGCTTAACACATGCAAGTCGA
ACGGGATCCATCGGGCTTTGCCTGGTGGTGAGAGTGGCGAACGGGTGAGTA
ATGCGTGACCGACCTGCCCCATGCACCGGAATAGCTCCTGGAAACGGGTGG
TAATGCCGGATGCTCCATCACACCGCATGGTGTGTTGGGAAAGCCTTTGCG
GCATGGGATGGGGTCGCGTCCTATCAGCTTGATGGCGGGGTAACGGCCCAC
CATGGCTTCGACGGGTAGCCGGCCTGAGAGGGCGACCGGCCACATTGGGAC
TGAGATACGGCCCAGACTCCTACGGGAGGCAGCAGTGGGGAATATTGCACA
ATGGGCGCAAGCCTGATGCAGCGACGCCGCGTGAGGGATGGAGGCCTTCGG
GTTGTAAACCTCTTTTGTTAGGGAGCAAGGCACTTTGTGTTGAGTGTACCT
TTCGAATAAGCACCGGCTAACTACGTGCCAGCAGCCGCGGTAATACGTAGG
GTGCAAGCGTTATCCGGAATTATTGGGCGTAAAGGGCTCGTAGGCGGTTCG
TCGCGTCCGGTGTGAAAGTCCATCGCTTAACGGTGGATCCGCGCCGGGTAC
GGGCGGGCTTGAGTGCGGTAGGGGAGACTGGAATTCCCGGTGTAACGGTGG
AATGTGTAGATATCGGGAAGAACACCAATGGCGAAGGCAGGTCTCTGGGCC
GTTACTGACGCTGAGGAGCGAAAGCGTGGGGAGCGAACAGGATTAGATACC
CTGGTAGTCCACGCCGTAAACGGTGGATGCTGGATGTGGGGCCCGTTCCAC
GGGTTCCGTGTCGGAGCTAACGCGTTAAGCATCCCGCCTGGGGAGTACGGC
CGCAAGGCTAAAACTCAAAGAAATTGACGGGGGCCCGCACAAGCGGCGGAG
CATGCGGATTAATTCGATGCAACGCGAAGAACCTTACCTGGGCTTGACATG
TTCCCGACGATCCCAGAGATGGGGTTTCCCTTCGGGGCGGGTTCACAGGTG
GTGCATGGTCGTCGTCAGCTCGTGTCGTGAGATGTTGGGTTAAGTCCCGCA
ACGAGCGCAACCCTCGCCCCGTGTTGCCAGCGGATTGTGCCGGGAACTCAC
GGGGGACCGCCGGGGTTAACTCGGAGGAAGGTGGGGATGACGTCAGATCAT
CATGCCCCTTACGTCCAGGGCTTCACGCATGCTACAATGGCCGGTACAACG
GGATGCGACAGCGCGAGCTGGAGCGGATCCCTGAAAACCGGTCTCAGTTCG
GATCGCAGTCTGCAACTCGACTGCGTGAAGGCGGAGTCGCTAGTAATCGCG
AATCAGCAACGTCGCGGTGAATGCGTTCCCGGGCCTTGTACACACCGCCCG
TCAAGTCATGAAAGTGGGCAGCACCCGAAGCCGGTGGCCTAACCCCTGCGG
GAGGGAGCCKC
SEQ ID NO: 2-5 - see Table 1

### REFERENCES

[1] Spor et al. (2011) Nat Rev Microbiol. 9(4):279-90.
[2] Eckburg et al. (2005) Science. 10;308(5728):1635-8.
[3] Macpherson et al. (2001) Microbes Infect. 3(12):1021-35
[4] Macpherson et al. (2002) Cell Mol Life Sci. 59(12):2088-96.
[5] Mazmanian et al. (2005) Cell 15;122(1):107-18.
[6] Frank et al. (2007) PNAS 104(34):13780-5.
[7] Scanlan et al. (2006) J Clin Microbiol. 44(11):3980-8.
[8] Kang et al. (2010) Inflamm Bowel Dis. 16(12):2034-42.
[9] Machiels et al. (2013) Gut. 63(8):1275-83.
[10] WO 2013/050792
[11] WO 03/046580
[12] WO 2013/008039
[13] WO 2014/167338
[14] Goldin and Gorbach (2008) Clin Infect Dis. 46 Suppl 2:S96-100.
[15] Azad et al. (2013) BMJ. 347:f6471.
[16] Olivares et al., (2014) British Journal of Nutrition 112(1), 30-40.
[17] Pinto-Sanchez et al., (2017) Journal of Clinical Gastroenterology 51(9), 814-817.
[18] Miraglia Del Giudice et al., (2017) Italian Journal of Pediatrics 43(1), 25.
[19] Groeger et al., (2013) Gut Microbes 4(4), 325-339. doi: 10.4161/gmic.25487.
[20] KR20100135612
[21] Coakley et al., (2006) Nutrition and Cancer, 56(1):95-102
[22] WO2010/143940
[23] WO2017/209156
[24] Matsuda et al., (2011) Vaccine, 29:1855-1858
[25] Masco et al. (2003) Systematic and Applied Microbiology, 26:557-563.
[26] Sr\dot{u}tková et al. (2011) J. Microbiol. Methods, 87(1):10-6.
[27] https://www.immunology.org/policy-and-public-affairs/briefings-and-position-statements/immunodeficiency
[28] Marciano and Holland (2017) Review article, Front. Immunol., https://doi.org/10.3389/fimmu.2017.00937
[29] Ren and Tories (2009) Brain Res Rev.; 60(1):57-64
[30] Martinon et al. (2002) Mol Cell.; 10(2):417-26.
[31] Gaur and Aggarwal (2003). Biochem Pharmacol; 66(8):1403-8.
[32] Wang and Lin (2008) Acta Pharmacol Sin.; 29(11): 1275-1288.
[33] Bloch et al. (2016) Eur Cytokine Netw.; 27(3):63-67
[34] Dowling and Mansell (2016) Clin Transl Immunology. 2016 May; 5(5): e85.
[35] Weinberger (2018) Curr Opin Pharmacol, 41, 34-41.
[36] Lim (2015) Aging Cell 14, 907-915
[37] Li et al, (2007) J Immunol, 178(8), 5271-5276
[38] Park et al., (2002) J Immunol, 169(3), 1433-1443
[39] Berthoud et al. (2009) J Immunol Methods 340(1) 33-41
[40] Mori et al. (2012), Eur J Immunol 42, 2709-2719
[41] Lai et al. (2018) Leukemia, 32(3):801-808.
[42] Barnes et al. (2015) J Immunother Cancer. 2015; 3: 1.
[43] Glenn and Whartenby (2014) World J Stem Cells.; 6(5): 526-539.
[44] Heng et al. (2004) Cardiovasc Res. 2004 Apr. 1; 62(1):34-42.
[45] Rashidi et al. (2018) Biology of Blood and Marrow Transplantation 24, Issue 6, Pages 1260-1263
[46] Fulop et al (2013) Crit Rev Oncog. 2013;18(6):489-513.
[47] Bektas et al. (2017) J Leukoc Biol.; 102(4):977-988.
[48] Fulop et al (2016) Rev Invest Clin.; 68(2):84-91.
[49] Fulop et al. (2018) Front Immunol; 8:1960.
[50] Miyamoto-Shinohara et al. (2008) J. Gen. Appl. Microbiol., 54, 9-24.
[51] Cryopreservation and Freeze-Drying Protocols, ed. by Day and McLellan, Humana Press.
[52] Leslie et al. (1995) Appl. Environ. Microbiol. 61, 3592-3597.
[53] Mitropoulou et al. (2013) J Nutr Metab. (2013) 716861.
[54] Kailasapathy et al. (2002) Curr Issues Intest Microbiol. 3(2):39-48.
[55] Handbook of Pharmaceutical Excipients, 2nd Edition, (1994), Edited by A Wade and P J Weller
[56] Remington's Pharmaceutical Sciences, Mack Publishing Co. (A. R. Gennaro edit. 1985)
[57] US 2016/0067188
[58] Handbook of Microbiological Media, Fourth Edition (2010) Ronald Atlas, CRC Press.
[59] Maintaining Cultures for Biotechnology and Industry (1996) Jennie C. Hunter-Cevera, Academic Press
[60] Strobel (2009) Methods Mol Biol. 581:247-61.
[61] Gennaro (2000) Remington: The Science and Practice of Pharmacy. 20th edition, ISBN: 0683306472.
[62] Molecular Biology Techniques: An Intensive Laboratory Course, (Ream et al., eds., 1998, Academic Press).
[63] Methods In Enzymology (S. Colowick and N. Kaplan, eds., Academic Press, Inc.)
[64] Handbook of Experimental Immunology, Vols. I-IV (D.M. Weir and C. C. Blackwell, eds, 1986, Blackwell Scientific Publications)
[65] Sambrook et al. (2001) Molecular Cloning: A Laboratory Manual, 3rd edition (Cold Spring Harbor Laboratory Press).
[66] Handbook of Surface and Colloidal Chemistry (Birdi, K. S. ed., CRC Press, 1997)
[67] Ausubel et al. (eds) (2002) Short protocols in molecular biology, 5th edition (Current Protocols).
[68] PCR (Introduction to Biotechniques Series), 2nd ed. (Newton & Graham eds., 1997, Springer Verlag)
[69] Current Protocols in Molecular Biology (F. M. Ausubel et al., eds., 1987) Supplement 30
[70] Smith & Waterman (1981) Adv. Appl. Math. 2: 482-489
[71] Sambrook et al., 1989
[72] Untergasser et al., (2007) Nucleic Acids Res 35(Web Server issue), W71-74. doi: 10.1093/nar/gkm306.
[73] Heberle et al., (2015) BMC Bioinformatics 16(1), 169. doi: 10.1186/s12859-015-0611-3.
[74] Ruiz et al. (2016) Front Microbiol 7, 1193. doi: 10.3389/fmicb.2016.01193.
[75] Fanning et al. (2012) Proc Natl Acad Sci USA 109(6), 2108-2113
[76] Christiaen et al., (2014) PLoS One 9(5), e98111. doi: 10.1371/journal.pone.0098111
[77] McCarville et al., (2017) Applied and Environmental Microbiology 83(19), e01323-01317. doi: 10.1128/AEM.01323-17.
[78] Cummings et al., (2014) Fems Microbiology Letters 351(2), 116-125. doi: 10.1111/1574-6968.12365.
[79] Bergonzelli et al., (2006) Infect Immun 74(1), 425-434. doi: 10.1128/IAI.74.1.425-434.2006.
[80] Candela et al., (2007) J Bacteriol 189(16), 5929-5936. doi: 10.1128/JB.00159-07
[81] Kinoshita et al., (2008) J Appl Microbiol 104(6), 1667-1674. doi: 10.1111/j.1365-2672.2007.03679.x.
[82] Candela et al., (2009) Microbiology 155(Pt 10), 3294-3303. doi: 10.1099/mic.0.028795-0.
[83] Candela et al., (2010) Microbiology 156(6), 1609-1618.
[84] Gonzalez-Rodriguez et al., (2012) Appl Environ Microbiol 78(11), 3992-3998. doi: 10.1128/AEM.08024-11.
[85] Hytonen et al., (2003) Infection and Immunity 71(2), 784-793.
[86] Hytonen et al., (2006) BMC Microbiol 6, 18. doi: 10.1186/1471-2180-6-18
[87] Wei et al., (2014) J Proteomics 108, 89-98. doi: 10.1016/j jprot.2014.04.038.
[88] O'Connell Motherway et al., (2009) Microbial Biotechnology 2(3), 321-332. doi: 10.1111/j.1751-7915.2008.00071.
[89] Bottacini et al., (2018) Scientific Reports 8(1), 10633. doi: 10.1038/s41598-018-28919-4
[90] Begley et al., (2006) Applied and Environmental Microbiology 72(3), 1729-1738.
[91] O'Connell Motherway et al., (2011) Proc Natl Acad Sci USA 108(27), 11217-11222.
[92] McCarville et al., (2017) Applied and Environmental Microbiology 83(19), e01323-01317.
[93] Rockwell et al., (1972) J Natl Cancer Inst. 49:735-49.
[94] Bertram and Jarvik (1980) Cancer Lett. 11:63-73.
[95] Darlington (1987) Meth Enzymol. 151:19-38.
[96] Principe d'éthique de l'expérimentation animale, Directive n°2010/63 CEE 22nd Sep. 2010, Décrêt n°2013-118 1 Feb. 2013.
[97] Guide for the Care and Use of Laboratory Animals: Eighth Edition. The National Academies Press; 2011
[98] Simpson-Herren and Lloyd (1970) Cancer Chemother Rep. 54:143-74.
[99] Workman et al. (2010) Br. J. Cancer. 102:1555-77.
[100]Goering (2010) Infect Genet Evol 10, 866-875.
[101]McCartney et al., (1996) Appl Environ Microbiol 62, 4608-4613.
[102]Roy (1996) Int J Food Microbiol 29, 11-29.
[103]Simpson, et al., (2003) J Clin Microbiol 33, 2233-2239.
[104]Bourget et al., (1993) FEMS Microbiol Lett 110, 11-20.
[105]Bottacini et al., (2015) Appl Environ Microbiol 81, 166-176
[106]Li et al. (2007) J Bacteriol 189, 6128-6139
[107]Tenover et al. (1995) J Clin Microbiol 33, 2233-2239
[108]Law et al., (1995) Journal of Bacteriology 177(24), 7011-7018.
[109]Wang and Kushner, (1991) Gene 100, 195-199.
[110]de Ruyter et al., (1996) Applied and Environmental Microbiology 62(10), 3662-3667.
[111]Alvarez-Martin et al., (2007) Applied Microbiology and Biotechnology 76(6), 1395.
[112]Cronin et al., (2007) Applied and Environmental Microbiology 73(24), 7858-7866.
[113]O'Connell Motherway et al., (2013) MicrobialBiotechnology 6(1), 67-79.
[114]Aziz et al., 2008 BMC Genomics 9, 75. doi: 10.1186/1471-2164-9-75.
[115]Zheng et al., (2014) PLoS One 9(5), e95441. doi: 10.1371/journal.pone.0095441.
[116]Overbeek et al., (2005) Nucleic Acids Research 33(17), 5691-5702.
[117]Yu et al., (2010) Bioinformatics 26(13), 1608-1615.

1.-30. (canceled)

31. A method of enhancing an immune response to an antigen in a subject in need thereof comprising administering to the subject a composition comprising a bacterial strain of the species Bifidobacterium breve, wherein the bacterial strain comprises a 16s rRNA gene sequence having at least 95% sequence identity to the sequence of SEQ ID NO: 1.

32. The method of claim 31, wherein the antigen comprises a viral antigen, a bacterial antigen, a fungal antigen, a parasite antigen, or a tumor antigen.

33. The method of claim 32, wherein the viral antigen is an antigen of a virus selected from the group consisting of influenza virus, human immunodeficiency virus (HIV), hepatitis B virus, herpes simplex virus, rabies, respiratory syncytial virus, cytomegalovirus, a coronavirus, varicella zoster virus, Epstein Barr virus, and human papillomavirus.

34. The method of claim 31, wherein the subject has a disease caused by a pathogen from which the antigen is derived.

35. The method of claim 31, wherein the subject has a disease caused by a coronavirus.

36. The method of claim 31, wherein the bacterial strain expresses EPS on its surface.

37. The method of claim 31, wherein the bacterial strain expresses pullulanase.

38. The method of claim 31, wherein the bacterial strain has a biochemical profile that is positive for fermentation of one or more of α-galactosidase, β-galactosidase, α-glucosidase and β-glucosidase, α-arabinose, mannose, and raffinose as determined by an Analytical Profile Index test.

39. The method of claim 31, wherein the immune response comprises an immune response to a vaccine comprising the antigen.

40. The method of claim 31, wherein the bacterial strain stimulates TLR2 or NFκB and/or increases the expression level and/or activity of IL-12p70, IL-12p70, IFNγ, IL-4, TNF-α and/or IL-17α.

41. The method of claim 31, wherein the bacterial strain comprises a 16s rRNA gene sequence having at least 98% sequence identity to the sequence of SEQ ID NO: 1, as determined by a Smith-Waterman homology search algorithm using an affine gap search with a gap open penalty of 12 and a gap extension penalty of 2.

42. The method of claim 31, wherein the bacterial strain comprises a 16s rRNA gene sequence of SEQ ID NO: 1.

43. The method of claim 31, wherein the bacterial strain is the strain deposited under accession number 42380 at NCIMB.

44. The method of claim 31, wherein the sequence identity is determined by a Smith-Waterman homology search algorithm using an affine gap search with a gap open penalty of 12 and a gap extension penalty of 2.

45. A method of treating an immunodeficiency in a subject in need thereof comprising administering to the subject a composition comprising a bacterial strain of the species Bifidobacterium breve, wherein the bacterial strain comprises a 16s rRNA gene sequence having at least 95% sequence identity to the sequence of SEQ ID NO: 1.

46. The method of claim 45, wherein the immunodeficiency comprises a primary immunodeficiency disease or a secondary immunodeficiency disease.

47. The method of claim 46, wherein the primary immunodeficiency disease comprises X linked agammaglobulinemia (XLA), chronic granulomatous disease (CGD), common variable immunodeficiency (CVID), or severe combined immunodeficiency (SCID).

48. The method of claim 46, wherein the secondary immunodeficiency disease comprises AIDS, a cancer of the immune system, or an immune-complex disease.

49. A method of stimulating the immune system in a subject in need thereof comprising administering to the subject a composition comprising a bacterial strain of the species Bifidobacterium breve, wherein the bacterial strain comprises a 16s rRNA gene sequence having at least 95% sequence identity to the sequence of SEQ ID NO: 1, wherein the subject has a disease or a condition that is treatable by stimulating the immune system.

50. The method of claim 49, wherein the disease or the condition is caused by a coronavirus.


SEQUENCE LISTING (5 sequences)

SEQ ID NO: 1 - DNA, 1439 bp, Bifidobacterium breve
gggacaggctcaggatgaacgccggcggcgtgcttaacacatgcaagtcgaacgggatccatcgggctttgcctggtggtgagagtggcgaacgggtgagtaatgcgtgaccgacctgccccatgcaccggaatagctcctggaaacgggtggtaatgccggatgctccatcacaccgcatggtgtgttgggaaagcctttgcggcatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgttagggagcaaggcactttgtgttgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacagcgcgagctggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaacccctgcgggagggagcckc

SEQ ID NO: 2 - DNA, 49 bp, Artificial Sequence
  Primer
cgtccgtctagaataaggaggcactcaccatgaataagcagcagcttgc

SEQ ID NO: 3 - DNA, 34 bp, Artificial Sequence
  Primer
gctctactcgaggcgatatgaggcgagcttcacg

SEQ ID NO: 4 - DNA, 18 bp, Artificial Sequence
  Primer
caggcattgaaggaatcg

SEQ ID NO: 5 - DNA, 32 bp, Artificial Sequence
  Primer
ctggctaagcttgtgcatgggctcagtccttc